(19) United States
(12) Patent Application Publication
(10) Pub. No.: US 2026/0347449 A1
(43) Pub. Date: Nov. 5, 2026

(54) NOVEL COMPLEMENT SYSTEM INHIBITING ANTIBODIES

(71) Applicant: 4D MOLECULAR THERAPEUTICS INC., Emeryville, CA (US)

(72) Inventors: TIMOTHY TSENG-HUI CHEN, Emeryville, CA (US); SHILPA MAROJU, Emeryville, CA (US); MELISSA CALTON, Emeryville, CA (US); ROXANNE CROZE, Emeryville, CA (US); ALBENA KANTARDZHIEVA, Emeryville, CA (US)

(73) Assignee: 4D MOLECULAR THERAPEUTICS INC., Emeryville, CA (US)

(21) Appl. No.: 19/485,517

(22) PCT Filed: May 22, 2024
(86) PCT No.: PCT/US2024/030542

§ 371 Date: Nov. 18, 2025

Related U.S. Application Data
(60) Provisional application No. 63/503,748, filed on May 23, 2023.
(60) Provisional application No. 63/503,768, filed on May 23, 2023.

(51) Int. Cl. C07K 16/18; A61K 48/00; C12N 15/86

(52) U.S. Cl. CPC C07K 16/18; A61K 48/005; C12N 15/86; C12N 2750/14143

(57) ABSTRACT

Novel anti-C3 and anti-C5 antibodies capable of modulating complement activity by specifically binding to human complement factor C3, C3a, C3b, and/or C5 are provided. Also described herein are recombinant AAV (rAAV) comprising a variant adeno-associated virus (AAV) capsid and a transgene encoding an anti-C3 and/or anti-C5 antibody are provided. Also provided are methods of delivering the transgene to the retina and methods of treating dry age-related macular degeneration and geographic atrophy secondary to age-related macular degeneration disorders by contacting retinal cells with the rAAV)

### CROSS REFERENCE TO RELATED APPLICATIONS

This application claims the benefit of U.S. Provisional Patent Application Ser. Nos. 63/503,745 and 63/503,768, both filed May 23, 2023, the entire contents of each of which are incorporated herein by reference.

### SEQUENCE LISTING SUBMISSION VIA EFS-WEB

A computer readable XML file, entitled “090400-5023-WO Sequence Listing” created on May 21, 2024, with a file size of about 249,282 bytes contains the sequence listing for this application and is hereby incorporated by reference in its entirety.

### BACKGROUND OF THE INVENTION

A number of human diseases are caused by complement dysregulation, resulting in complement-mediated autologous tissue injury. The complement dysregulation may arise from mutations, either somatic or germline, in complement regulator or regulator-related genes such that these regulators no longer function normally. In particular, there are common and rare human diseases that are caused by excessive complement activation resulting from dysregulation of the complement activation cascade.

Current therapeutic approaches are focused on the development of reagents such as monoclonal antibodies (mAbs), peptides or other small molecules that bind and block specific alternative pathway or terminal pathway complement components. A clinically validated example is Eculizumab, a humanized mAb against complement C5 which has been approved for the treatment of paroxysmal nocturnal hemoglobinuria (PNH) and atypical hemolytic uremic syndrome (aHUS). Other approaches that have been described include mAbs against factor B (fB), factor D (fD), or properdin (fP), and a cyclic peptide that binds and inhibits C3. A limitation of these approaches is that they require repeated and inconvenient intravenous (IV) dosing of patients. Further, since these treatments block the alternative pathway or terminal pathway, they run the risk of compromising host defense. Indeed, patients on Eculizumab therapy have to be vaccinated against bacteria strains that cause lethal meningitis and these patients are also put on prophylactic antibiotic therapy before being treated with the approved mAb drug.

In other approaches, recombinant regulatory proteins such as soluble DAF, CR1, CRIg and proteins comprising minimal domains of fluid phase regulator fH (N-terminal short consensus repeat [SCR] 1-5 and C-terminal SCR 19-20) or fusion proteins between fH and CR2 (TT30) have been tested. However, large scale heterologous expression of such proteins as therapeutic drugs requires significant effort, and animal studies have shown their in vivo clearance rate after administration to be fast making such therapeutic strategies cumbersome and less practical as multiple and frequent administrations of such protein drugs would be required.

A need remains in the art for compositions useful for treating complement-mediated diseases with greater and longer-lasting efficacy.

### SUMMARY OF THE INVENTION

Described herein are polypeptides comprising antibodies, preferably comprising a single variable domain on a heavy chain (VHH or nanobody) capable of modulating complement activity by specifically binding to human complement factor C3, C3a, C3b, C5, C5a and/or C5b. Preferably, the polypeptide comprises a VH or VHH domain of, or derived from, a camelid heavy chain antibody.

In some aspects, the VH or VHH domain comprises one or more complementarity determining regions (CDRs) as set forth in Tables 1 and 2. In preferred aspects, the VH or VHH domain comprises the CDRS (CDR1, CDR2 and CDR3) of one or more antibodies selected from the group consisting of SEQ ID Nos: 1-14 and SEQ ID Nos: 57-65. In other preferred aspects, said CDR1, CDR2 and CDR3 are separated by frame regions FR1, FR2, FR3, and FR4 of the VH or VHH domain.

In some preferred aspects, the VH or VHH domain comprises or consists of any one of the amino acid sequences set forth as SEQ ID Nos: 1-14 or any one of the amino acid sequences set forth as SEQ ID Nos: 57-65.

In other preferred aspects, the VH or VHH domain is humanized and comprises or consists of any one of amino acid sequences set forth as SEQ ID Nos: 94-136 or any one of the amino acid sequences set forth as SEQ ID Nos: 137-163.

In other preferred aspects, the VH or VHH domain comprises a tag, such as a Strep or his6 tag. In other aspects, the VH or VHH domain is fused to another polypeptide.

In other embodiments, a polynucleotide (DNA or RNA) is provided, wherein said polynucleotide comprises a nucleotide sequence that encodes a polypeptide comprising one or more CDRs of a VH or VHH domain as herein described, or encodes a polypeptide comprising a VH or VHH domain as herein described. In related embodiments, an expression vector comprising said polynucleotide is provided, wherein said nucleotide sequence is operably linked to an expression control sequence (e.g., a promoter). In other embodiments, a pharmaceutical composition comprising a polypeptide or expression vector as described herein and a pharmaceutically acceptable carrier is provided.

Also provided are recombinant AAV (rAAV) virions comprising a variant AAV capsid sequence encapsulating a heterologous nucleic acid comprising a nucleotide sequence encoding an anti-C3 and/or anti-C5 antibody as herein described. Preferably, the nucleotide sequence encoding the antibody is operably linked to an expression control sequence.

The variant AAV capsid protein of the rAAV comprises a capsid protein comprising a peptide insertion of from about 7 amino acids to about 20 amino acids (a “heterologous peptide” or “peptide insertion”) in the GH-loop of the capsid protein, preferably in a surface-exposed region of the GH-loop, relative to a corresponding parental AAV capsid protein, wherein the peptide insertion comprises the amino acid sequence ISDQTKH (SEQ ID NO:168). In some preferred aspects, the peptide insertion comprises one to three spacer amino acids (Y₁-Y₃) at the amino and/or carboxy terminal of the amino acid sequence ISDQTKH (SEQ ID NO:168), wherein each of Y₁-Y₃ is independently selected from Ala, Leu, Gly, Ser, Thr, and Pro. In particularly preferred embodiments, the peptide insertion comprises, consists essentially of or consists of the amino acid sequence LAISDQTKHA (SEQ ID NO:169). In certain preferred embodiments, the peptide is inserted following any of the amino acids in positions 584-591 in VP1 of AAV2 or a corresponding position in another AAV serotype (i.e., the insertion site is between amino acids 587 and 588 of VP1 of AAV2 or is between amino acids 588 and 589, between amino acids 584 and 585, between amino acids 585 and 586, between amino acids 586 and 587, between amino acids 590 and 591, or is between amino acids 591 and 592 of AAV2 or the corresponding positions in the capsid protein of another AAV serotype). In some embodiments, the capsid protein further comprises one or more amino acid substitutions relative to VP1 capsid of AAV2 or one or more corresponding substitutions in another AAV serotype, preferably wherein the capsid protein further comprises a P34A amino acid substitution relative to VP1 capsid of AAV2 or the corresponding substitution in another AAV serotype.

In other embodiments, a method for delivering a heterologous nucleic acid comprising a nucleotide sequence encoding an antibody as herein described to a mammalian subject is provided, the method comprising administering to the mammal an effective amount of an rAAV as herein described or a pharmaceutical composition comprising same, preferably wherein the rAAV or pharmaceutical composition is administered by intravitreal injection. In some aspects, the heterologous nucleic acid is delivered to a retinal cell of the subject, e.g., a photoreceptor cell (e.g., rods; cones), a retinal ganglion cell (RGC), a glial cell (e.g., a Müller glial cell, a microglial cell), a bipolar cell, an amacrine cell, a horizontal cell, and/or a retinal pigmented epithelium (RPE) cell of the subject.

In some embodiments, detectable plasma levels of the antibody are present in a subject for at least a week, at least two weeks, at least three weeks, at least a month, at least two months, or at least 6 months following administration of the rAAV to the subject. In particularly preferred embodiments, the rAAV is administered to a subject by intravitreal administration.

In other embodiments, a pharmaceutical composition is provided comprising an rAAV as described herein and a pharmaceutically acceptable excipient.

In other aspects, a method is provided for treating a complement related disorder by delivering to the subject an rAAV as herein described or a pharmaceutical composition comprising the rAAV. Complement related disorders that may be treated include, without limitation, membranoproliferative glomerulonephritis, atypical hemolytic uremic syndrome (aHUS), age related macular degeneration (AMD), geographic atrophy secondary to AMD microangiopathic haemolytic anemia, thrombocytopenia, acute renal failure, paroxysmal nocturnal hemoglobinuria (PNH), schizophrenia, ischemic stroke, and/or bacterial infections caused by recruitment of bacterial pathogens.

In a further aspect, a method is provided for treating dry age-related macular degeneration (AMD) (e.g., late-stage dry AMD) in a subject in need thereof by delivering to the subject an effective amount of an rAAV as herein described or a pharmaceutical composition comprising the rAAV. In related aspects, the rAAV or pharmaceutical composition is administered to a subject for the treatment of geographic atrophy secondary to AMD. Preferably, the rAAV or pharmaceutical composition is administered to the subject by intravitreal injection.

### DESCRIPTION OF THE DRAWINGS

FIGS. 1A-C Induced pluripotent stem cell derived retinal pigmented epithelial cells (iPSC-RPE) were transduced with C3-N10 rAAV transgene (encoding anti-C3 antibody of SEQ ID NO: 14) at three multiplicities of infection, (MOI) 5,000, 10,000 and 20,000. A dose response was observed for nanobody (VHH) expression, (FIG. 1A), and MAC formation (FIG. 1B). Immunocytochemistry depicts complement membrane attack complex (MAC) deposition in iPSC-RPE following Zymosan (a potent activator of the alternative complement pathway), and inhibition of MAC deposition following C3-N10 rAAV transduction (FIG. 1C).

FIGS. 2A-C Complement Inhibition by Wild Type Lead Nanobodies in Alternative, Lectin & Classical Complement Pathways. All three wild type nanobodies' complement inhibition activity were compared using Wieslab's alternative, MBL/lectin, and classical complement pathway assay kits. As shown in FIG. 2A (alternative), FIG. 2B (lectin) and FIG. 2C (classical), while C5S2 has similarly strong inhibitory potency against all three pathways, C3S3 only exhibits activity against the alternative pathway. C3N10 appears to have stronger inhibitory affect towards the alternative pathway, milder potency against the lectin and classical pathways.

FIGS. 3A-C IC50 Potency of Wild Type and Humanized Nanobodies against Alternative Complement Pathway. To assess potency differences between wild type and their humanized counterparts, each nanobody was serially diluted and test with the Wieslab alternative complement pathway kit and their IC50s calculated using GraphPad Prism. As shown for anti-C3 (FIGS. 3A and 3B) and anti-5C nanobodies (FIG. 3C), all humanized versions retain similar IC50 activity with their original wild types.

FIGS. 4A-B IC50 potency of C3N10 and C3N10.3 against classical (FIG. 4A) and lectin (FIG. 4B) pathways. To compare the inhibitory activity between wild type and humanized versions of C3N10 within the classical and lectin complement pathways, these nanobodies were serial diluted and the samples tested using the respective Wieslab assay kits. Both versions have similar IC50 potencies against those tested pathways.

FIG. 5 illustrates Cross Reactivity of Nanobody Wild Type Leads with Mouse Serum, Murine cross-inhibitory activity of the wild type nanobodies was examined using Hycult's C3 Mouse ELISA Kit and the mouse-anti-mouse C3 antibody BB5.1 as control. None of the anti-human C3 or C5 nanobodies, C3N10, C3S3, nor C582 exhibited inhibitory activity against the mouse serum mediated alternative complement pathway.

FIGS. 6A-B IC50 of wild type and humanized nanobodies against alternative complement pathway in cynomolgus serum. Both wild type and their humanized counterparts were serial diluted and their cynomolgus serum mediated alternative complement pathway inhibitory activity was calculated using the respective Wieslab assay. C3N10 WT and C3N10.3 (FIG. 6A) along with C3S3 WT and C3S3.2 (FIG. 6B) showed activity against cynomolgus alternative complement pathway, with similar IC50s between each variant. C5S2 and C5S2.2 did not exhibit any inhibitory activity in this NHP serum mediated assay (data not shown).

FIG. 7 Functional potency of anti-C3 and C5 VHH-Fc Dimers in Alternative Complement Pathway in Human Serum. Humanized anti-C3 and C5 nanobodies were reengineered as G2-hinge-G4-Fc fusion proteins and their IC50s measured by serial dilutions in the Wieslab alternative complement pathway assay. Data was graphed and IC50s calculated using GraphPad Prism. (CS52.2-Fc IC_{50 MAC}=9.076 nM; C3S3-Fc_{50 MAC}=23.80 nM; C3N10.3-Fc_{50 MAC}=49.31 nM)

FIG. 8 Amino acid interactions between C3N10.3 to human C3 protein. Humanized C3N10.3 was allowed to associate with human C3 protein and cross-linking mass spectrometry (CovalX) was performed to identify potential binding peptide epitopes. Three epitopes within amino acids 1540-600 of human C3 protein were identified (amino acids 1548-1551, 1568-1573 and 1583-1591).

FIG. 9 Amino acid interactions between nanobody C3S3.2 to human C3 protein. Humanized C3S3.2 was allowed to associate human C3 protein and cross-linking mass spectrometry (CovalX) was performed to identify potential binding peptide epitopes. An epitope between amino acids 110-130 was identified (amino acids 114-124) and another epitope between amino acids 490-510 (amino acids 497-502 and 502-505) of human C3 protein was identified.

FIG. 10 illustrates potential binding domains of C3N10.3 and C3S3.2 to human C3 protein. Based upon the cross-linking mass spectrometry data, the binding regions of C3N10.3 and C3S3.2 to human C3 protein are shown, wherein the former associates within the alpha-chain's C345C, while the latter interacts across both MG1 and MG5 domains of the beta-chain, respectively.

FIG. 11 shows Eculizumab and C5S2.2 cross block binding to human C5: Probe anti-human Fc Sandwich. The Gator/BLI machine was used to examine whether C5S2.2 cross blocks eculizumab, a clinically approved anti-human C5 targeting hIgG4 antibody. Anti-human Fc probes were pre-bound by eculizumab, then washed. Then, either human C5-alone or increasing concentrations of human C5 pre-bound with C5S2.2 were allowed to associate with the antibody-loaded probes. Binding kinetics was only observed with free C5 protein indicating that the pre-bound C5S2.2 VHH was blocking eculizumab's C5 binding epitope. Time (sec) is on the X-axis and Shift (nm) is on the Y-axis.

FIG. 12 shows Eculizumab and C5S2.2 cross block binding to human C5: Probe anti-StepII sandwich. The Gator/BLI machine was used to examine whether C5S2.2 cross blocks eculizumab, a clinically approved anti-human C5 targeting hIgG4 antibody. Anti-Strep II probes were pre-bound with Strep-tagged anti-human C5 C5S2.2 VHH. After washing, the nanobody loaded probes were dipped into human C5 containing binding buffer and association kinetics was observed. When this pre-bound complex was then dipped into eculizumab containing solution, no interaction was seen, implicating that the latter's binding epitope was blocked. Time (sec) is on the X-axis and Shift (nm) is on the Y-axis.

FIGS. 13A-C show C5S2.2 binding to human and cynomolgus C5 mutants. Eculizumab is refractory to two naturally occurring variants: the R885H human polymorphism and the W917S human vs cynomolgus divergence. Human C5 R885H, human C5 W917S, and cynomolgus C5 S917W mutant proteins were recombinantly produced and C5S2.2's affinity to them was compared to eculizumab (FIGS. 14A-C) using the Gator.

FIGS. 14A-C shows Eculizumab binding to human and cynomolgus C5 mutants. Eculizumab is refractory to two naturally occurring variants: the R885H human polymorphism and the W917S human vs cynomolgus divergence. Human C5 R885H, human C5 W917S, and cynomolgus C5 S917W mutant proteins were recombinantly produced and eculizumab's affinity to them was reflected using the Gator

FIG. 15 shows percentage of normal human donor's anti-drug antibody (ADA) response to wild-type and humanized C3 and C5 nanobodies (Cap=caplacizumab/Cablivi; Beovu-brolucizumab). Normal human donor serum was tested for ADA against the listed nanobodies and controls using meso-scale discovery immunoassays. Samples were diluted 1:100 and applied to plate wells passively coated with the respective antibodies. Bound human antibodies were detected using Sulfo-TAG goat-anti-human IgG and measured in a Meso Sector S 600.

FIGS. 16A-C show functional assessment of VHH expressed following plasmid transfection by breakdown products of complement cascade associated proteins. FIG. 16A: C3 VHH inhibitors, but not C5 VHH inhibitors, prevent C3 cleavage as detected by absence of C3a breakdown product. FIG. 16B: C3 VHH inhibitors and C5 VHH inhibitors prevent C5 cleavage as detected by absence of C5a breakdown product. FIG. 16C: C3 VHH inhibitors, but not C5 VHH inhibitors, prevent Bb cleavage as detected by absence of Factor B breakdown products. v, version; h, humanized; kDa, kilodalton; NT, non-transfected.

FIG. 17 is a Western blot demonstrating a single product for each VHH variant examined following transduction with AAV carrying VHH. NT, non-transduced; kDa, kilodalton; MOI 20,000; 33 μg protein loaded per lane; seven days post transduction.

FIGS. 18A-C show expression of secreted VHH (C3N10 and C3N10 humanized, FIG. 18A; C3S3 and humanized C3S3, FIG. 18B; C5S2 and humanized C5S2, FIG. 18C), following AAV transduction, as measured by ELISA and illustrating a dose response observed for each VHH (MOIs of 1,000, 5,000 and 20,000). NT, non-transduced; MOI, multiplicity of infection; error bars±standard deviation; n=3 wells/MOI.

FIG. 19 shows function of secreted VHH (C3N10, C3N10 h, C353, C353 h, C5S2, C5S2 h) as measured by complement inhibition represented as a percent. Each VHH exhibited some level of complement inhibition. NT, non-transduced; h, humanized; MOI, multiplicity of infection; error bars±standard deviation; n=3 wells/MOI.

### DETAILED DESCRIPTION OF THE INVENTION

### Definitions

An “antibody” is used herein in the broadest sense to refer to a polypeptide or protein with an immunoglobulin-like domain capable of recognizing and binding an antigen and includes full-size antibodies, individual chains thereof and all portions, domains or fragments thereof (including but not limited to, antigen binding domains or fragments such as VHH domains or VH/VL domains, respectively). The antigen binding site of an antibody preferably comprises at least one complementarity determining region (CDR). The present disclosure relates primarily to single variable domain on a heavy chain (VHH) antibodies. As used herein, the terms “single domain antibody”, “single variable domain antibody”, “VHH antibody”, and “nanobody” have the same meaning referring to a variable region of a heavy chain of an antibody, and construct a single domain antibody consisting of only one heavy chain variable region. Thus, the antigen binding site of a single variable domain is formed by no more than three CDRs. Generally, the antibodies with a natural deficiency of the light chain and the heavy chain constant region 1 (CH1) are first obtained, the variable regions of the heavy chain of the antibody are therefore cloned to construct a single domain antibody (VHH) consisting of only one heavy chain variable region. The VHH antibody is preferably derived from llama.

An “antigen” is a molecule comprising at least one epitope. The antigen may for example be a polypeptide, nucleic acid, polysaccharide, protein, lipoprotein or glycoprotein.

A “complementarity determining region” or “CDR” is a hypervariable region of the antigen-binding region of an antibody. The CDRs are interspersed between regions that are more conserved, termed framework regions (FRs). The antigen-binding region of an antibody may thus comprise one or more CDRs and FRs, usually in each variable domain three CDRs and four FRs arranged from amino-terminus to carboxy-terminus in the following order: FR1, CDR1, FR2, CDR2, FR3, CDR3, FR4.

An “epitope” is a determinant capable of specific binding to an antibody. Epitopes may for example be comprised within polypeptides or proteins. Epitopes may be continuous or discontinuous, wherein a discontinuous epitope is a conformational epitope on an antigen which is formed from at least two separate regions in the primary sequence of the protein, nucleic acid or polysaccharide.

The term “affinity” refers to the strength of binding between an antibody and its antigen. The affinity of an antibody can be defined in terms of the dissociation constant, KD, which is an equilibrium constant that measures the propensity of a molecular complex to separate (dissociate) reversibly into the molecules forming the complex. In one aspect, K_{D} is defined as the ratio k_{Off}/kₒₙ, where k_{Off} and kₒₙ are the rate constants for association and dissociation of the molecular complex. Preferably affinity is determined by calculating the dissociation constant K_{D} based on IC50 values. Thus, the affinity is measured as an apparent affinity.

The term “isolated” designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then that a single vector can contain just a single coding region, or comprise two or more coding regions.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

As used herein, the term “nucleic acid” is interchangeable with “polynucleotide” or “nucleic acid molecule” and a polymer of nucleotides is intended.

A polynucleotide which encodes a gene product, e.g., a polypeptide, can include a promoter and/or other transcription or translation control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other transcription control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit beta-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A polynucleotide or polypeptide has a certain percent “sequence identity” to another polynucleotide or polypeptide, meaning that, when aligned, that percentage of bases or amino acids are the same when comparing the two sequences. As described in aspects of the invention, sequence identity is related to sequence homology. Homology comparisons may be conducted by eye, or more usually, with the aid of readily available sequence comparison programs. Sequence similarity or sequence homology can be determined in a number of different manners. Commercially available computer programs may calculate percent (%) homology between two or more sequences and may also calculate the sequence identity shared by two or more amino acid or nucleic acid sequences. Sequence homologies may be generated by any of a number of computer programs known in the art, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. See J. Mol. Biol. 48:443-453 (1970).

% homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity.

However, these more complex methods assign “gap penalties” to each gap that occurs in the alignment so that, for the same number of identical amino acids, a sequence alignment with as few gaps as possible-reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system.

High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p 387). Examples of other software than may perform sequence comparisons include, but are not limited to, the BLAST package (see Ausubel et al., 1999 Short Protocols in Molecular Biology, 4th Ed.—Chapter 18), FASTA (Altschul et al., 1990 J Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health).

Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

| | Set | Sub-set | |
|---|---|---|---|
| Hydrophobic | F W Y H K M I L | Aromatic | F W Y H |
| | V A G C | Aliphatic | I L V |
| Polar | W Y H K R E D C | Charged | H K R E D |
| | S T N Q | Positively charged | H K R |
| | | Negatively charged | E D |
| Small | V C A G S P T N D | Tiny | A G S |

Embodiments of the invention include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, which involves the presence of one or more amino acid residues in peptoid form, may be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term “amino acid substitution” and its synonyms described above are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease (and/or symptoms caused by the disease) from occurring in a subject which may be predisposed to the disease or at risk of acquiring the disease but has not yet been diagnosed as having it; (b) inhibiting the disease (and/or symptoms caused by the disease), i.e., arresting its development; and (c) relieving the disease (and/or symptoms caused by the disease), i.e., causing regression of the disease (and/or symptoms caused by the disease), i.e., ameliorating the disease and/or one or more symptoms of the disease.

As used herein, the term “treating complement factor H disorders” may encompass alleviating, reducing, and/or ameliorating symptoms, and/or preventing the development of additional symptoms associated with complement factor H disorder, which can manifest as several different phenotypes, including asymptomatic, recurrent bacterial infections, and renal failure. This is typically characterized by decreased serum levels of factor H, complement component C3, and a decrease in other terminal complement components, indicating activation of the alternative complement pathway. Serum levels of complement component C5 may also be decreased. This disorder is associated with a number of renal diseases with variable clinical presentation and progression, including C3 glomerulopathy and atypical hemolytic uremic syndrome. Also provided herein are compositions and methods for treating one or more of dry age related macular degeneration (AMD), geographic atrophy secondary to AMD, atypical hemolytic uremic (including, e.g., syndrome microangiopathic haemolytic anemia, thrombocytopenia, acute renal failure), paroxysmal nocturnal hemoglobinuria (PNH), schizophrenia, ischemic stroke, and/or preventing or treating bacterial infections caused by recruitment of bacterial pathogens (e.g., Aspergillus spp.; Borrelia burgdorferi; B. duttonii; B. recurrentis; Candida albicans; Francisella tularensis; Haemophilus influenzae; Neisseria meningitidis; Streptococcus pyogenes, or one of the five factor H binding proteins of B. burgdorferi (CRASP-1, CRASP-2, CRASP-3, CRASP-4, or CRASP-5), among others.

As used herein, the term “treating complement associated disorders” includes alleviating, reducing, and/or ameliorating symptoms, both of the complement factor H disorders identified above, but also other disorders associated with uncontrolled alternative pathway complement regulation.

“Complement-mediated disorders” may encompass symptoms associated with complement dysregulation which can manifest as several different phenotypes, including asymptomatic, recurrent bacterial infections, and various tissue injuries including but not limited to renal diseases. Unless otherwise specified, both homozygous subjects and heterozygous subjects are encompassed within this definition. Complement dysregulation is typically caused by loss of function mutations in, or auto-antibodies against, complement regulatory proteins including but not limited to fH, factor I (fI) and membrane cofactor protein (MCP) or by gain of function mutations in other complement proteins including but not limited to C3, C5 and factor B (fB). Complement dysregulation is typically, though not always, characterized by decreased serum levels of factor H, complement component C3, fB and a decrease in other terminal complement components, indicating activation of the alternative and/or the terminal complement pathway. Complement-mediated pathologies that can be treated by the present invention of composition and method include but are not limited to the following diseases with variable clinical presentation and progression: C3 glomerulopathy (formally called membranoproliferative glomerulonephritis type II or MPGNII), of which there are two known forms-dense deposit disease (DDD) and C3 glomerulonephritis (C3GN); thrombotic microangiopathy (TMA) including but not limited to atypical hemolytic uremic syndrome (aHUS), Shiga-like toxin-producing E. coli HUS (STEC-HUS) and thrombotic thrombocytopenia purpura (TTP); retinal degenerative eye disease including age related macular degeneration (AMD), RPE degeneration, chorioretinal degeneration, photoreceptor degeneration, paroxysmal nocturnal hemoglobinuria (PNH), ischemia reperfusion injury of all organs and settings, rheumatoid arthritis, hemodialysis, diabetic nephropathy, diabetic vasculopathy, asthma, systemic lupus erythematosus (SLE), ischemic stroke, abdominal aortic aneurysm (AAA), anti-neutrophil cytoplasmic antibody (ANCA) mediated vasculitis (ANCA vasculitis), ANCA-mediated hemorrhagic lung injury and disease, ANCA glomerulonephritis, graft versus host disease (GvHD), acute or delay graft rejection in organ transplantation, Crohn's disease, psoriasis, multiple sclerosis, anti-phospholipid syndrome, preeclampsia, atherosclerosis, neuromyelitis optica (NMO), autoimmune skin-blistering disease, Bullous pemphigoid (BP), Alzheimer's disease (AD), as well as bacterial infections caused by recruitment of bacterial pathogens (e.g., Aspergillus spp.; Borrelia burgdorferi; B. duttonii; B. recurrentis; Candida albicans; Fr and sella tularensis; Haemophilus influenzae; Neisseria meningitidis; Streptococcus pyogenes).

The terms “individual,” “host,” “subject,” and “patient” are used interchangeably herein, and refer to a mammal, including, but not limited to, primates (e.g., humans; non-human primates, including simians); mammalian sport animals (e.g., horses); mammalian farm animals (e.g., sheep, goats, etc.); mammalian pets (dogs, cats, etc.); and rodents (e.g., mice, rats, etc.).

The term “effective amount” as used herein is an amount sufficient to effect beneficial or desired clinical results. An effective amount can be administered in one or more administrations. For purposes of this disclosure, an effective amount of a compound (e.g., an infectious rAAV virion) is an amount that is sufficient to palliate, ameliorate, stabilize, reverse, prevent, slow or delay the progression of (and/or symptoms associated with) a particular disease state (e.g., a disorder associated with complement dysfunction). Accordingly, an effective amount of an infectious rAAV virion is an amount of the infectious rAAV virion that is able to effectively deliver a heterologous nucleic acid to a target cell (or target cells) of the individual. Effective amounts may be determined preclinically by, e.g., detecting in the cell or tissue the gene product (RNA, protein) that is encoded by the heterologous nucleic acid sequence using techniques that are well understood in the art, e.g. RT-PCR, western blotting, ELISA, fluorescence or other reporter readouts, and the like. Effective amounts may be determined clinically by, e.g. detecting a change in the onset or progression of disease using methods known in the art, e.g. 6-minute walk test, left ventricular ejection fraction, hand-held dynamometry, Vignos Scale and the like as described herein and as known in the art.

### DETAILED DESCRIPTION

Novel anti-C3 and anti-C5 antibodies and nucleic acids encoding same are described herein. rAAV virions comprising a nucleic acid encoding the anti-C3 and/or antibodies are also described. These rAAV virions are characterized by durable and robust expression of anti-C3 and anti-C5 antibodies in the retina and increased efficacy in treating conditions associated with C3 and C5 and other complement disorders.

Delivery of these rAAV virions to subjects in need thereof may be achieved via a number of routes, preferably by intravitreal administration. Also provided are methods of using these rAAV virions in regimens for treating complement factor C3- and C5-associated disorders, particularly dry AMD and geographic atrophy secondary to AMD.

### Antibodies

Naturally occurring human antibodies are heterotetramers. The antibodies provided herein in one aspect comprise an antigen binding site in a single polypeptide. The antibodies are therefore herein referred to as “single variable domain” or “VHH” antibodies. Single variable domain antibodies are also known as nanobodies. The single variable domain antibodies disclosed herein may, though, in certain embodiments, be bispecific or multispecific single variable domain antibodies, in which single variable domain antibodies are coupled.

A single variable domain antibody is an antibody fragment consisting of a single monomeric variable antibody domain. Like a whole antibody, it is able to bind selectively to a specific antigen. Single variable domain antibodies typically have molecular weights in the range of 12-15 kDa, i.e. much lower than common antibodies, ranging typically from 150 to 160 kDa. Single variable domain antibodies are also smaller than Fab fragments (~50 kDa) of heterotetrameric antibodies comprising one light chain and half a heavy chain.

Single domain antibodies can derive from antibodies found in nature, for example in camelids (VHH) and cartilaginous fishes (VNAR). New or Nurse Shark Antigen Receptor (NAR) protein exists as a dimer of two heavy chains with no associated light chains. Each chain is composed of one variable (V) and five constant domains. The NAR proteins thus constitute a single immunoglobulin variable-like domain. Single heavy-chain antibodies are also found in camelids, such as such as dromedaries, camels, llamas and alpacas, where the heavy chain has lost one of its constant domains and underwent modifications in the variable domain, both of which are structural elements necessary for the binding of light chains.

However, single variable domain antibodies can also be engineered by recombinant methods. One approach is to split the dimeric variable domains from common immunoglobulin G (IgG) from humans or mice into monomers. Single domains, which are derived from light chains, also bind specifically to target epitopes. Thus, the single variable domain antibody may be derived from any suitable organism.

Single domain camelid antibodies are equal to regular antibodies in terms of specificity. Single domain antibodies are easily isolated, for example by using phage panning procedures. The smaller size and single domain architecture make these antibodies easier to express as proteins in bacterial cells for large scale production, making them ideal for commercial exploitation. The antibodies of the present invention are therefore single variable domain antibodies, preferably derived from camelid antibodies, preferably llama antibodies, including functional homologs, fragments thereof and fusion macromolecules containing VHH covalently linked to glycan, nucleic acid, protein, or chemical groups not being a macromolecule.

In certain aspects, an antibody provided herein is an antibody fragment. In one aspect, the antibody fragment is a Fab, Fab′, Fab′-SH, or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

Antibody fragments can be made by various techniques, including but not limited to proteolytic digestion of an intact antibody as well as recombinant production by recombinant host cells (e.g., E. coli, CHO).

In a preferred embodiment the antibody provided herein is a Fab fragment. In one embodiment the VH domain of the antibody provided herein comprises a human VH3 framework. In one embodiment the VL domain of the antibody provided herein comprises a human Vkappa1 framework. In one embodiment the CL domain of the antibody provided herein is of kappa isotype. In one embodiment the CH1 domain of the antibody provided herein is of human IgG1 isotype.

In a preferred embodiment, the antibody provided herein is a Fab fragment comprising a CL domain of kappa isotype and a CH1 domain of human IgG1 isotype.

In certain aspects, an antibody provided herein is a multispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities.

Multispecific antibodies with three or more binding specificities comprising antibodies provided herein may be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). Various further molecular formats for multispecific antibodies are known in the art and are included herein (see e.g., Spiess et al., Mol Immunol 67 (2015) 95-106).

In certain aspects, amino acid sequence variants of the antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain aspects, antibody variants having one or more amino acid substitutions are provided. Sites of interest for substitutional mutagenesis include the CDRs and FRs. Conservative substitutions are shown below under the heading of “preferred substitutions”. More substantial changes are provided below under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more CDR residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies, Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4): 680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and Cyto Tox 960 non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402, To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol, 18 (12): 1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FeRs are described. (See, e.g., U.S. Pat. No. 6,737,056; WO 2004/056312, and Shields et al., J. Biol. Chem. 9 (2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG1Fc region. In one aspect, the substitutions are L234A, L235A and P329G (LALA-PG) in an Fc region derived from a human IgG1 Fc region. (See, e.g., WO 2012/130831). In another aspect, the substitutions are L234A, L235A and D265A (LALA-DA) in an Fc region derived from a human IgG1 Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fe variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol, 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fe-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fe region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG1 Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### Immunoconjugates

The invention also provides immunoconjugates comprising an antibody provided herein conjugated (chemically bonded) to one or more agents; in one embodiment such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one embodiment the invention provides immunoconjugates comprising an antibody provided herein conjugated to a polymer. The term “polymer” used herein includes chemical polymers and protein polymers. In one embodiment the immunoconjugate comprises the antibody provided herein conjugated to an extended recombinant polypeptide (XTEN). “Extended recombinant polypeptides” are known in the art and are, e.g. disclosed in US20190083577. In one embodiment the immunoconjugate comprises an XTEN (a) comprising a sequence selected from GGSPAGSCTSP, GASASCAPSTG, TAEAAGCGTAEAA, and GPEPTCPAPSG. (b) being 36 to 3000 L-amino acid residues in length, and/or (c) wherein the sum of glycine (G), alanine (A), serine(S), threonine (T), glutamate (E) and proline (P) residues constitutes more than 90% of the total amino acid residues of the XTEN.

### Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In one aspect, isolated nucleic acids encoding an antibody of the invention are provided. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified. In one embodiment the host cell is an E. coli cell.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell). In one preferred embodiment the host cell is a CHO cell. Production of antibodies of the invention in CHO cells may improve syringeability of the antibody.

### Anti-C3 Antibodies

In some aspects, an anti-C3 antibody as herein described is capable of specifically binding to an epitope of human complement factor C3 and/or proteolytic derivatives C3a and/or C3b. Thus, an anti-C3 antibody is capable of specifically binding to an epitope in any region of the polypeptide sequence identified by GenBank Accession No. NP_000055.2, the entire contents of which are incorporated herein by reference.

The antibodies described herein comprise one or more CDRs. In particular, the CDRs may identify the specificity of the antibody and accordingly it is preferred that the antigen binding site comprises at least 2, and more preferably comprises 3 CDRs.

Thus, the antibodies provided herein are preferably derived from natural antibodies, such as camelid antibodies. In some aspects, the antibody is selected from the following sequences or sequences at least 75% identical thereto (CDR sequences are underlined):

C3S1;
(SEQ ID NO: 1)
QVQLQESGGGLVQAGGSLRLSCAASGNISAPSLMGWYRQAPGKEREFVAT
ITYGSITNYVDSVKGRFTISRDNAKNTVYLQMNSLKPEDTAVYYCAANTL
GIYNTYGDHHYWGQGTQVTVSS
C3S3:
(SEQ ID NO: 2)
QVQLQESGGGLVQAGGSLRLSCAASGTIFSRNYMGWYRQAPGKERELVAG
IGYGSSTNYADSVKGRFTISRDNAKNTVYLQMNSLKPEDTAVYYCAAVPT
YRPYYFYWGQGTQVTVSS
C3S4:
(SEQ ID NO: 3)
QVQLQESGGGLVQAGGSLRLSCAASGTISGPGDMGWYRQAPGKERELVAG
INYGAITYYADSVKGRFTISRDNAKNTVYLQMNSLKPEDTAVYYCAADYE
TWAPSYEYFDYWGQGTQVTVSS
C3S5:
(SEQ ID NO: 4)
QVQLQESGGGLVQAGGSLRLSCAASGTIFDDESMGWYRQAPGKEREFVAS
INYGSTTYYADSVKGRFTISRDNAKNTVYLQMNSLKPEDTAVYYCAVGWS
TNYDWTLSYYYWGQGTLVTVSS
C3S6:
(SEQ ID NO: 5)
QVQLQESGGGLVQAGGSLRLSCAASGNIFVGHGMGWYRQAPGKEREFVAA
IGYGAITNYADSVKGRFTISRDNAKNTVYLQMNSLKPEDTAVYYCAASSY
YPPRYWYWGQGTLVTVSS
C3S7:
(SEQ ID NO: 6)
QVQLQESGGGLVQAGGSLRLSCAASGYIFTGVDMGWYRQAPGKEREFVAA
ISYGASTYYADSVKGRFTISRDNAKNTVYLQMNSLKPEDTAVYYCAASLS
NTYLLDLFFRYWGQGTQVTVSS
C3S8:
(SEQ ID NO: 7)
QVQLQESGGGLVQAGGSLRLSCAASGTIFPTDRMGWYRQAPGKEREFVAT
IDLGGTTNYADSVKGRFTISRDNAKNTVYLQMNSLKPEDTAVYYCAAAWE
GPSRYGRHRYYSHRYWGQGTQVTVSS
C3S9:
(SEQ ID NO: 8)
QVQLQESGGGLVQAGGSLRLSCAASGYIFVYSTMGWYRQAPGKEREFVAS
INYGSITYYADSVKGRFTISRDNAKNTVYLQMNSLKPEDTAVYYCAVTDK
SRYWLTYWGQGTQVTVSS
C3S10:
(SEQ ID NO: 9)
QVQLQESGGGLVQAGGSLRLSCAASGYISGGSIMGWYRQAPGKEREFVAG
INQGSNTYYADSVKGRFTISRDNAKNTVYLQMNSLKPEDTAVYYCAVGGR
SYYYHHYWGQGTQVTVSS
C3I3:
(SEQ ID NO: 10)
DVQLVESGGGLVQPGGSLRLSCAASGFTLDNYAIAWFRQAPGKEREGVSC
ISSSRGSTAYEDSVKGRFTIARDNAKNTVYLQMNSLKPEDTAVYYCAARA
RGMCREFDYDYWGQGTQVTVSS
C3I4:
(SEQ ID NO: 11)
DVQLVESGGGLVQPGGSLRLSCATSGFTLDYYAIGWFRQAPGKEREGVSC
ITGPDGSTHYADSVKGRFTISRDNAKNTVYLQMNSLKPEDTAVYYCAATA
GLYGCGYGMDHWGKGTQVTVSS
C3I8:
(SEQ ID NO: 12)
DVQLVESGGGLVQPGGSLRLSCATSGFSFGVYDMSWVRQASGKGLEWVSG
IDSGSNTAYADSVKVRFTISRDNAKNTLYLQMNNLKPEDTAVYYCALGRE
TDYIPVTDFRTQGTQVTVSS
C3I22:
(SEQ ID NO: 13)
DVQLVESGGGLVQPGGSLRLSCTASGFTFGVYAMSWVRQAPGKGLEWVSS
IDSGVNTAYADSVKGRFTISRDNAINTVFLQMNSLKPEDTAVYHCALGRE
SDYEPVTGSWGQGTQVTVSS
C3N10:
(SEQ ID NO: 14)
DVQLVESGGGLVQAGGSLRLACAASGLTFSPYAMGWFRQAPGKEREFVAG
IRWISGNPYYADSVKGRFTISRDNAKNTVYLQMNSLKPEDTAVYSCAAAP
LTRSYDYWGQGTQVTVSS

CDRs of the above-described antibodies are listed below at Table 1:

TABLE 1

| Antibody | CDR1 | CDR2 | CDR3 |
|---|---|---|---|
| C3S1 | GNISAPSLM | TITYGSITNY | NTLGIYNTYGDHHY |
| | (SEQ ID NO: 15) | (SEQ ID NO: 16) | (SEQ ID NO: 17) |
| C3S3 | GTIFSRNYM | GIGYGSSTNY | VPTYRPYYFY |
| | (SEQ ID NO: 18) | (SEQ ID NO: 19) | (SEQ ID NO: 20) |
| C3S4 | GTISGPGDMG | GINYGAITYY | DYETWAPSYEYFDY |
| | (SEQ ID NO: 21) | (SEQ ID NO: 22) | (SEQ ID NO: 23) |
| C3S5 | GTIFDDESMG | SINYGSTTYY | GWSTNYDWTLSYYY |
| | (SEQ ID NO: 24) | (SEQ ID NO: 25) | (SEQ ID NO: 26) |
| C3S6 | GNIFVGHGMG | AIGYGAITNY | SSYYPPRYWY |
| | (SEQ ID NO: 27) | (SEQ ID NO: 28) | (SEQ ID NO: 29) |
| C3S7 | GYIFTGVDMG | AISYGASTYY | SLSNTYLLDLFFRY |
| | (SEQ ID NO: 30) | (SEQ ID NO: 31) | (SEQ ID NO: 32) |
| C3S8 | GTIFPTDRMG | TIDLGGTTNY | AWEGPSRYGRHRYYSHRY |
| | (SEQ ID NO: 33) | (SEQ ID NO: 34) | (SEQ ID NO: 35) |
| C3S9 | GYIFVYSTMG | SINYGSITYY | TDKSRYWLTY |
| | (SEQ ID NO: 36) | (SEQ ID NO: 37) | (SEQ ID NO: 38) |
| C3S10 | GYISGGSIMG | GINQGSNTYY | GGRSYYYHHY |
| | (SEQ ID NO: 39) | (SEQ ID NO: 40) | (SEQ ID NO: 41) |
| C3SI3 | GFTLDNYAIA | CISSSRGSTAY | RARGMCREFDYDY |
| | (SEQ ID NO: 42) | (SEQ ID NO: 43) | (SEQ ID NO: 44) |
| C3SI4 | GFTLDYYAIG | CITGPDGSTHY | TAGLYGCGYGMDH |
| | (SEQ ID NO: 45) | (SEQ ID NO: 46) | (SEQ ID NO: 47) |
| C318 | GFSFGVYDMS | GIDSGSNTAY | GRETDYIPVTDF |
| | (SEQ ID NO: 48) | (SEQ ID NO: 49) | (SEQ ID NO: 50) |
| C3I22 | GFTFGVYAMS | SIDSGVNTAY | GRESDYEPVTGS |
| | (SEQ ID NO: 51) | (SEQ ID NO: 52) | (SEQ ID NO: 53) |
| C3N10 | GLTFSPYAMG | GIRWISGNPYY | APLTRSYDY |
| | (SEQ ID NO: 54) | (SEQ ID NO: 55) | (SEQ ID NO: 56) |

In some embodiments, the antibody comprises a CDR having at least one, at least two, or at least three amino acid differences relative to a CDR as set forth in Table 1. In some embodiments, the antibody comprises a CDR1 having the following sequence: G-F/L-T/S-F/L-S/G/D-P/V/Y/N-Y-A/D-M/I-G/S/A (wherein the first amino acid is G, the second amino acid is F or L, etc.) and/or comprises a CDR2 having the following sequence: G/S/C-I-R/D/T/S/N-W/S/G/Q-I/G/P/S-S/V/D/R-G/N-N/T/S-P/A/T/Y-Y/H/A-Y/H, wherein the 11^{th} amino acid is optional.

In related embodiments, the antibody comprises a CDR1, CDR2 and/or CDR3 selected from the group consisting of the CDR sequences set forth at Table 1.

In some preferred embodiments, the antibody comprises a CDR1 selected from SEQ ID Nos: 15, 18, 21, 24, 27, 30, 33, 36, 39, 42, 45, 48, 51, and 54, or comprises a CDR1 comprising a sequence at least 75% identical to any one of SEQ ID Nos: 15, 18, 21, 24, 27, 30, 33, 36, 39, 42, 45, 48, 51, and 54; and/or comprises a CDR2 selected from SEQ ID Nos: 16, 19, 22, 25, 28, 31, 34, 37, 40, 43, 46, 49, 52, and 55 or comprises a CDR2 comprising a sequence at least 75% identical to any one of SEQ ID Nos: 16, 19, 22, 25, 28, 31, 34, 37, 40, 43, 46, 49, 52, and 55; and/or comprises a CDR3 selected from SEQ ID Nos: 17, 20, 23, 26, 29, 32, 35, 38, 41, 44, 47, 50, 53, and 56 or comprises a CDR1 comprising a sequence at least 75% identical to any one of SEQ ID Nos: 17, 20, 23, 26, 29, 32, 35, 38, 41, 44, 47, 50, 53, and 56. In a related embodiment, the antibody comprises a CDR1, CDR2, and CDR3 selected from: SEQ ID Nos: 15-17, 18-20, 21-23, 24-26, 27-29, 30-32, 33-35, 36-38, 39-41, 42-44, 45-47, 48-50, 51-53 and 54-56 or comprises a CDR1, CDR2 and CDR3 at least 75% identical to SEQ ID Nos: 15-17, 18-20, 21-23, 24-26, 27-29, 30-32, 33-35, 36-38, 39-41, 42-44, 45-47, 48-50, 51-53 and 54-56.

In a particularly preferred embodiment, the antibody comprises a CDR1, CDR2 and CDR3 of SEQ ID Nos: 54-56, respectively, or comprises CDRs at least 75% (e.g., at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97% or at least 98%) identical thereto.

The amino acid sequence of cynomolgus C3 is below:

(SEQ ID NO: 176)
MGLTSGPSLLLLLLIHLPLALGTPMYSMITPNVLRLESEETVVLE
AHDANGDVPVTVTVHDFPGKKLVLSSEKTVLTPATSHMGSVTIRI
PANKEFKSEKGHNKFVTVQATFGAQVVEKVVLVSLQSGYLFIQTD
KTIYTPGSTVLCRIFTVNHKLLPVGRTVVVNIENPDGIPVKQDSL
SSQNQFGILPLSWDIPELVNMGQWKIRAYYENSPQQVFSTEFEVK
EYVLPSFEVIVEPTEKFYYIYNQKGLEVTITARFLYGKKVEGTAF
VIFGIQDGEQRISLPESLKRIQIEDGSGDAVLSRKVLLDGVQNPR
PEDLVGKSLYVSVTVILHSGSDMVQAERSGIPIVTSPYQIHFTKT
PKYFKPGMPFDLMVFVTNPDGSPAYRVPVAVQGEDAVQSLTQGDG
VAKLSINTHPSQKPLSITVRTKKRELSEAEQATRTMEAQPYSTVG
NSNNYLHLSVPRAELRPGETLNVNFLLRMDRTQEAKIRYYTYLIM
NKGKLLKVGRQVREPGQDLVVLPLSITTDFIPSFRLVAYYTLIGA
NGQREVVADSVWVDVKDSCVGSLVVKSGQSEDRQPLPGQQMTLKI
EGDHGARVGLVAVDKGVFVLNKKNKLTQSKIWDVVEKADIGCTPG
SGKDYAGVFSDAGLTFASSSGQQTAQRAELQCPQPAARRRRSVQL
AEKRMDKVGQYPKELRKCCEHGMRENPMRFSCQRRTRYITLDEAC
KKAFLDCCNYITELRRQHARASHLGLARSNLDEDIIAEENIVSRS
EFPESWLWKIEELKEAPKNGISTKLMNIFLKDSITTWEILAVSLS
DKKGICVADPFEVTVMQDFFIDLRLPYSVVRNEQVEIRAVLYNYR
QNQELKVRVELLHNPAFCSLATAKRRHQQTVTIPPKSSLSVPYVI
VPLKTGQQEVEVKAAVYHFFISDGVRKSLKVVPEGIRMNKTVAVR
TLDPERLGQEGVQREDVPPADLSDQVPDTESETRILLQGTPVAQM
TEDAIDAERLKHLIVTPSGCGEQNMITMTPTVIAVHYLDETEQWE
KFGPEKRQGALELIKKGYTQQLAFRQPSSAFAAFLNRAPSTWLTA
YVVKVFSLAVNLIAIDSQVLCGAVKWLILEKQKPDGVFQEDAPVI
HQEMTGGFRNTNEKDMALTAFVLISLQEAKEICEEQVNSLPGSIT
KAGDFLEANYMNLQRSYTVAIAAYALAQMGRLKGPLLNKFLTTAK
DKNRWEEPGQQLYNVEATSYALLALLQLKDFDFVPPVVRWLNEQR
YYGGGYGSTQATFMVFQALAQYQKDVPDHKELNLDVSLQLPSRSS
KIIHRIHWESASLLRSEETKENEGFTVTAEGKGQGTLSVVTMYHA
KAKGQLTCNKFDLKVTIKPAPETEKRPQDAKNTMILEICTRYRGD
QDATMSILDISMMTGFVPDTDDLKQLANGVDRYISKYELDKAFSD
RNTLIIYLDKVSHSEDDCIAFKVHQYFNVELIQPGAVKVYAYYNL
AESCTRFYHPEKEDGKLNKLCRDELCRCAEENCFIQKLDDKVTLE
ERLDKACEPGVDYVYKTRLVKAQLSNDFDEYIMAIEQIIKSGSDE
VQVGQQRTFISPIKCREALKLEERKHYLMWGLSSDFWGEKPNLSY
IIGKDTWVEHWPEEDECQDEENQKQCQDLGTFTENMVVFGCPN

The amino acid sequence of human C3 is provided below:

(SEQ ID NO: 177)
MGPTSGPSLLLLLLTHLPLALGSPMYSIITPNILRLESEETMVLE
AHDAQGDVPVTVTVHDFPGKKLVLSSEKTVLTPATNHMGNVTFTI
PANREFKSEKGRNKFVTVQATFGTQVVEKVVLVSLQSGYLFIQTD
KTIYTPGSTVLYRIFTVNHKLLPVGRTVMVNIENPEGIPVKQDSL
SSQNQLGVLPLSWDIPELVNMGQWKIRAYYENSPQQVFSTEFEVK
EYVLPSFEVIVEPTEKFYYIYNEKGLEVTITARFLYGKKVEGTAF
VIFGIQDGEQRISLPESLKRIPIEDGSGEVVLSRKVLLDGVQNPR
AEDLVGKSLYVSATVILHSGSDMVQAERSGIPIVTSPYQIHFTKT
PKYFKPGMPFDLMVFVTNPDGSPAYRVPVAVQGEDTVQSLTQGDG
VAKLSINTHPSQKPLSITVRTKKQELSEAEQATRTMQALPYSTVG
NSNNYLHLSVLRTELRPGETLNVNFLLRMDRAHEAKIRYYTYLIM
NKGRLLKAGRQVREPGQDLVVLPLSITTDFIPSFRLVAYYTLIGA
SGQREVVADSVWVDVKDSCVGSLVVKSGQSEDRQPVPGQQMTLKI
EGDHGARVVLVAVDKGVFVLNKKNKLTQSKIWDVVEKADIGCTPG
SGKDYAGVFSDAGLTFTSSSGQQTAQRAELQCPQPAARRRRSVQL
TEKRMDKVGKYPKELRKCCEDGMRENPMRFSCQRRTRFISLGEAC
KKVELDCCNYITELRRQHARASHLGLARSNLDEDIIAEENIVSRS
EFPESWLWNVEDLKEPPKNGISTKLMNIFLKDSITTWEILAVSMS
DKKGICVADPFEVTVMQDFFIDLRLPYSVVRNEQVEIRAVLYNYR
QNQELKVRVELLHNPAFCSLATTKRRHQQTVTIPPKSSLSVPYVI
VPLKTGLQEVEVKAAVYHHFISDGVRKSLKVVPEGIRMNKTVAVR
TLDPERLGREGVQKEDIPPADLSDQVPDTESETRILLQGTPVAQM
TEDAVDAERLKHLIVTPSGCGEQNMIGMTPTVIAVHYLDETEQW
EKFGLEKRQGALELIKKGYTQQLAFRQPSSAFAAFVKRAPSTWLT
AYVVKVFSLAVNLIAIDSQVLCGAVKWLILEKQKPDGVFQEDAPV
IHQEMIGGLRNNNEKDMALTAFVLISLQEAKDICEEQVNSLPGSI
TKAGDFLEANYMNLQRSYTVAIAGYALAQMGRLKGPLLNKFLTTA
KDKNRWEDPGKQLYNVEATSYALLALLQLKDFDFVPPVVRWLNEQ
RYYGGGYGSTQATFMVFQALAQYQKDAPDHQELNLDVSLQLPSRS
SKITHRIHWESASLLRSEETKENEGFTVTAEGKGQGTLSVVTMYH
AKAKDQLTCNKFDLKVTIKPAPETEKRPQDAKNTMILEICTRYRG
DQDATMSILDISMMTGFAPDTDDLKQLANGVDRYISKYELDKAFS
DRNTLIIYLDKVSHSEDDCLAFKVHQYFNVELIQPGAVKVYAYYN
LEESCTRFYHPEKEDGKLNKLCRDELCRCAEENCFIQKSDDKVTL
EERLDKACEPGVDYVYKTRLVKVQLSNDFDEYIMAIEQTIKSGSD
EVQVGQQRTFISPIKCREALKLEEKKHYLMWGLSSDFWGEKPNLS
YIIGKDTWVEHWPEEDECQDEENQKQCQDLGAFTESMVVFGCPN

In some aspects, an anti-C3 antibody as herein described (e.g. C3N10.3) binds to an epitope within the C3 alpha-chain at the C345C domain. In some aspects, an anti-C3 antibody as herein described binds to an epitope within amino acids 1540-1600 of human C3 or an amino acid sequence at least 80% identical thereto. In related aspects, an anti-C3 antibody as herein described binds to an epitope comprising amino acids 1548-1561 of human C3 or an amino acid sequence at least 80% identical thereto and/or binds to an epitope comprising amino acids 1568-1573 of human C3 or an amino acid sequence at least 80% identical thereto and/or binds to an epitope comprising amino acids 1583-1591 of human C3 or an amino acid sequence at least 80% identical thereto.

In some aspects, an anti-C3 antibody as herein described (e.g. C3S3.2) binds to at least one discontinuous epitope within the C3 beta-chain at the MG1 and MG5 domains. In some aspects, an anti-C3 antibody as herein described binds to at least one discontinuous epitope within amino acids 110-130 of human C3 and within amino acids 490-501 of human C3. In some aspects, an anti-C3 antibody as herein described binds to an epitope comprising amino acids 114-124 of human C3 or an amino acid sequence at least 80% identical thereto and/or binds to an epitope comprising amino acids 497-505 of human C3 or an amino acid sequence at least 80% identical thereto.

In some preferred embodiments, the anti-C3 antibody comprises a CDR1, CDR2 and/or CDR3 of the anti-C3 antibody of any one of SEQ ID Nos: 2, 14, 98 and 135 or comprises a CDR1, CDR2 and/or CDR3 at least 70% identical thereto (or comprising four or fewer amino acid substitutions relative to CDR1, CDR2, CDR3 of any one of SEQ ID Nos: 2, 14, 98 and 135).

### Anti-C5 Antibodies

In some aspects, an anti-C5 antibody (e.g., nanobody) as herein described is capable of specifically binding to an epitope of human complement factor C5 and/or proteolytic derivatives C5a and/or C5b. Thus, an anti-C5 antibody is capable of specifically binding to an epitope in any region of the polypeptide sequence identified by GenBank Accession No. NP_001304092.1, the entire contents of which are incorporated herein by reference.

The antibodies described herein comprise one or more CDRs. In particular, the CDRs may identify the specificity of the antibody and accordingly it is preferred that the antigen binding site comprises at least 2, and more preferably comprises 3 CDRs.

Thus, the antibodies provided herein are preferably derived from natural antibodies, such as camelid antibodies. In some aspects, the antibody is selected from the following sequences or sequences at least 75% identical thereto (CDR sequences are underlined):

C5S1:
(SEQ ID NO: 57)
QVQLQESGGGLVQAGGSLRLSCAASGYISYDDDMGWYRQAPGKEREFVAAIDVGGST
YYADSVKGRFTISRDNAKNTVYLQMNSLKPEDTAVYYCAVRDGYYWDHDYWGQGTQ
VTVSS
C5S2:
(SEQ ID NO: 58)
QVQLQESGGGLVQAGGSLRLSCAASGNIFHQSDMGWYRQAPGKEREFVAAIDYGINTY
YADSVKGRFTISRDNAKNTVYLQMNSLKPEDTAVYYCAARDTSYWDHVYWGQGTQV
TVSS
C5S3:
(SEQ ID NO: 59)
QVQLQESGGGLVQAGGSLRLSCAASGSISFDTDMGWYRQAPGKEREFVATIDGGTSTY
YADSVKGRFTISRDNAKNTVYLQMNSLKPEDTAVYYCAAEAGYFSLLGPYFRYWGQG
TQVTVSS
C5S4:
(SEQ ID NO: 60)
QVQLQESGGGLVQAGGSLRLSCAASGYIFAATDMGWYRQAPGKEREFVATIDYGANTN
YADSVKGRFTIGRDNAKNTVYLQMNSLKPEDTAVYYCAARDNNYYDLVYWGQGTQV
TVSS
C5S5:
(SEQ ID NO: 61)
QVQLQESGGGLVQAGGSLRLSCAASGTISPYEGMGWYRQAPGKERELVAAIDGGGITY
YADSVKGRFTISRDNAKNTVYLQMNSLKPEDTAVYYCAAGIWTLSYYDYWGQGTQVT
VSS
C5$6:
(SEQ ID NO: 62)
QVQLQESGGGLVQAGGSLRLSCAASGTIFYSYKMGWYRQAPGKEREFVAGITYGGSTY
YADSVKGRFTISRDNAKNTVYLQMNSLKPEDTAVYYCAARAYYVSVYDTALYYWGQ
GTQVTVSS
C5S7:
(SEQ ID NO: 63)
QVQLQESGGGLVQAGGSLRLSCAASGYIFYDDDMGWYRQAPGKEREFVATIDVGGNT
NYADSVKGRFTIGRDNAKNTVYLQMNSLKPEDTAVYYCAVRDNNYYDHVYWGQGTQ
VTVSS
C5S8:
(SEQ ID NO: 64)
QVQLQESGGGLVQAGGSLRLSCAASGNIFDEWDMGWYRQAPGKEREFVAAIDDGAST
YYADSVKGRFTISRDNAKNTVYLQMNSLKPEDTAVYYCAARQTYAWDYRYWGQGTQ
VTVSS
C5S12:
(SEQ ID NO: 65)
QVQLQESGGGLVQAGGSLRLSCAASGYIFPSDDMGWYRQAPGKEREFVAAINDGASTY
YADSVKGRFTISRDNAKNTVYLQMNSLKPEDTAVYYCAVQLNYYWDYVYWGQGTQV
TVSS

CDRs of the above-described antibodies are listed below at Table 2:

TABLE 2

| Antibody | CDR1 | CDR2 | CDR3 |
|---|---|---|---|
| CSS1 | GYISYDDDMG | AIDVGGSTY | RDGYYWDHDY |
| | (SEQ ID NO: 66) | (SEQ ID NO: 67) | (SEQ ID NO: 68) |
| C5S2 | GNIFHQSDMG | AIDYGTNTY | RDTSYWDHVY |
| | (SEQ ID NO: 69) | (SEQ ID NO: 70) | (SEQ ID NO: 71) |
| C5S3 | GSISFDTDMG | TIDGGTSTY | EAGYFSLLGPYFRY |
| | (SEQ ID NO: 72) | (SEQ ID NO: 73) | (SEQ ID NO: 74) |
| CSS4 | GYIFAATDMG | TIDYGANTN | RDNNYYDLVY |
| | (SEQ ID NO: 75) | (SEQ ID NO: 76) | (SEQ ID NO: 77 |
| C5S5 | GTISPYEGMG | AIDGGGITY | GIWTLSYYDY |
| | (SEQ ID NO: 78) | (SEQ ID NO: 79) | (SEQ ID NO: 80) |
| CSS6 | GTIFYSYKMG | GITYGGSTY | RAYYVSVYDTALYY |
| | (SEQ ID NO: 81) | (SEQ ID NO: 82) | (SEQ ID NO: 83) |
| C5S7 | GYIFYDDDMG | TIDVGGNTN | RDNNYYDHVY |
| | (SEQ ID NO: 84) | (SEQ ID NO: 85) | (SEQ ID NO: 86) |
| C5S8 | GNIFDEWDMG | AIDDGASTY | RQTYAWDYRY |
| | (SEQ ID NO: 87) | (SEQ ID NO: 88) | (SEQ ID NO: 89) |
| C5S12 | GYIFPSDDMG | AINDGASTY | QLNYYWDYVY |
| | (SEQ ID NO: 90) | (SEQ ID NO: 91) | (SEQ ID NO: 92) |

In some embodiments, the antibody comprises a CDR having at least one, at least two, or at least three amino acid differences relative to a CDR as set forth in Table 2. In some embodiments, the antibody comprises a CDR1 having the following sequence: G-Y/N/S/T-I-S/F-Y/H/F/A/P/D-D/Q/A/Y/S/E-D/S/T/E/Y/W-D/K/G-M-G (wherein the first amino acid is G, the second amino acid is Y, N, S or T, etc.) and/or comprises a CDR2 having the following sequence: A/T/G-I-D/T/N-V/Y/G/D-G-G/T/A-S/N/I-T-Y/N. In related embodiments, the antibody comprises a CDR1 having the following sequence: G-Y/N/S/T-I-F-Y/H/F/A/P/D-D/Q/A/Y/S/E-D/S/T/E/Y/W-D-M-G and/or comprises a CDR2 having the following sequence-A/T-I-D-V/Y/G/D-G-A-S/N-T-Y.

In related embodiments, the antibody comprises a CDR1, CDR2 and/or CDR3 selected from the group consisting of the CDR sequences set forth at Table 2.

In a preferred embodiment, the antibody comprises a CDR1 selected from SEQ ID Nos: 66, 69, 72, 75, 78, 81, 84, 87 and 90 or comprises a CDR1 comprising a sequence at least 75% identical to any one of SEQ ID Nos: 66, 69, 72, 75, 78, 81, 84, 87 and 90; and/or comprises a CDR2 selected from SEQ ID Nos: 67, 70, 73, 76, 79, 82, 85, 88 and 91 or comprises a CDR2 comprising a sequence at least 75% identical to any one of SEQ ID Nos: 67, 70, 73, 76, 79, 82, 85, 88 and 91; and/or comprises a CDR3 selected from SEQ ID Nos: 68, 71, 74, 77, 80, 83, 86, 89 and 92 or comprises a CDR1 comprising a sequence at least 75% identical to any one of SEQ ID Nos: 68, 71, 74, 77, 80, 83, 86, 89 and 92. In a related embodiment, the antibody comprises a CDR1, CDR2, and CDR3 selected from: SEQ ID Nos: 66-68, 69-71, 72-74, 75-77, 78-80, 81-83, 84-86, 87-89 and 90-92 or comprises a CDR1, CDR2 and CDR3 at least 75% identical to SEQ ID Nos: 66-68, 69-71, 72-74, 75-77, 78-80, 81-83, 84-86, 87-89 and 90-92.

The amino acid sequence of cynomolgus C5 is provided below:

(SEQ ID NO: 174)
MGLLGILCFLIFLGKTWGQEQTYVISAPKIFRVGASENIVIQVYGYTEAF
DATISIKSYPDKKFSYSSGHVHLSSENKFQNSAVLTIQPKQLPGGQNQVS
YVYLEVVSKHFSKSKKIPITYDNGFLFIHTDKPVYTPDQSVKVRVYSLND
DLKPAKRETVLTFIDPEGSEIDMVEEIDHIGIISFPDFKIPSNPRYGMWM
IQAKYKEDFSTTGTAFFEVKEYVLPHFSVSVEPESNFIGYKNFKNFEITI
KARYFYNKVVTEADVYITFGIREDLKDDQKEMMQTAMQNTMLINGIAEVT
FDSETAVKELSYYSLEDLNNKYLYIAVTVIESTGGFSEEAEIPGIKYVLS
PYKLNLVATPLFLKPGIPYSIKVQVKDALDQLVGGVPVTLNAQTIDVNQE
TSDLEPRKSVTRVDDGVASFVVNLPSGVTVLEFNVKTDAPDLPDENQARE
GYRAIAYSSLSQSYLYIDWTDNHKALLVGEYLNIIVTPKSPYIDKITHYN
YLILSKGKIIHFGTREKLSDASYQSINIPVTQNMVPSSRLLVYYIVTGEQ
TAELVSDSVWLNIEEKCGNQLQVHLSPDADTYSPGQTVSLNMVTGMDSWV
ALTAVDSAVYGVQRRAKKPLERVFQFLEKSDLGCGAGGGLNNANVFHLAG
LTFLTNANADDSQENDEPCKEIIRPRRMLQEKIEEIAAKYKHLVVKKCCY
DGVRINHDETCEQRAARISVGPRCVKAFTECCVVASQFRANNSHKDLQLG
RLHMKTLLPVSKPEIRSYFPESWLWEVHLVPRRKQLQFALPDSVTTWEIQ
GVGISNSGICVADTIKAKVFKDVFLEMNIPYSVVRGEQVQLKGTVYNYRT
SGMQFCVKMSAVEGICTSESPVIDHQGTKSSKCVRQKVEGSSNHLVTFTV
LPLEIGLQNINFSLETSFGKEILVKSLRVVPEGVKRESYSGITLDPRGIY
GTISRRKEFPYRIPLDLVPKTEIKRILSVKGLLVGEILSAVLSREGINIL
THLPKGSAEAELMSVVPVFYVFHYLETGNHWNIFHSDPLIEKRNLEKKLK
EGMVSIMSYRNADYSYSVWKGGSASTWLTAFALRVLGQVHKYVEQNQNSI
CNSLLWLVENYQLDNGSFKENSQYQPIKLQGTLPVEARENSLYLTAFTVI
GIRKAFDICPLVKINTALIKADTFLLENTLPAQSTFTLAISAYALSLGDK
THPQFRSIVSALKREALVKGNPPIYRFWKDSLQHKDSSVPNTGTARMVET
TAYALLTSLNLKDINYVNPIIKWLSEEQRYGGGFYSTQDTINAIEGLTEY
SLLVKQLRLNMDIDVAYKHKGPLHNYKMTDKNFLGRPVEVLLNDDLVVST
GFGSGLATVHVTTVVHKTSTSEEVCSFYLKIDTQDIEASHYRGYGNSDYK
RIVACASYKPSKEESSSGSSHAVMDISLPTGINANEEDLKALVEGVDQLF
TDYQIKDGHVILQLNSIPSSDFLCVRFRIFELFEVGFLSPATFTVYEYHR
PDKQCTMFYSTSNIKIQKVCEGATCKCIEADCGQMQKELDLTISAETRKQ
TACNPEIAYAYKVIITSITTENVFVKYKATLLDIYKTGEAVAEKDSEITF
IKKVTCTNAELVKGRQYLIMGKEALQIKYNFTFRYIYPLDSLTWIEYWPR
DTTCSSCQAFLANLDEFAEDIFLNGC

The amino acid sequence of human C5 is provided below:

(SEQ ID NO: 175)
MGLLGILCFLIFLGKTWGQEQTYVISAPKIFRVGASENIVIQVYGYTEAF
DATISIKSYPDKKFSYSSGHVHLSSENKFQNSAILTIQPKQLPGGQNPVS
YVYLEVVSKHFSKSKRMPITYDNGFLFIHTDKPVYTPDQSVKVRVYSLND
DLKPAKRETVLTFIDPEGSEVDMVEEIDHIGHISFPDFKIPSNPRYGMWT
IKAKYKEDFSTTGTAYFEVKEYVLPHFSVSIEPEYNFIGYKNFKNFEITI
KARYFYNKVVTEADVYITFGIREDLKDDQKEMMQTAMQNTMLINGIAQVT
FDSETAVKELSYYSLEDLNNKYLYIAVTVIESTGGFSEEAEIPGIKYVLS
PYKLNLVATPLFLKPGIPYPIKVQVKDSLDQLVGGVPVTLNAQTIDVNQE
TSDLDPSKSVTRVDDGVASFVLNLPSGVTVLEFNVKTDAPDLPEENQARE
GYRAIAYSSLSQSYLYIDWTDNHKALLVGEHLNIIVTPKSPYIDKITHYN
YLILSKGKIIHFGTREKFSDASYQSINIPVTQNMVPSSRLLVYYIVTGEQ
TAELVSDSVWLNIEEKCGNQLQVHLSPDADAYSPGQTVSLNMATGMDSWV
ALAAVDSAVYGVQRGAKKPLERVFQFLEKSDLGCGAGGGLNNANVFHLAG
LTFLTNANADDSQENDEPCKEILRPRRTLQKKIEEIAAKYKHSVVKKCCY
DGACVNNDETCEQRAARISLGPRCIKAFTECCVVASQLRANISHKDMQLG
RLHMKTLLPVSKPEIRSYFPESWLWEVHLVPRRKQLQFALPDSLTTWEIQ
GVGISNTGICVADTVKAKVFKDVFLEMNIPYSVVRGEQIQLKGTVYNYRT
SGMQFCVKMSAVEGICTSESPVIDHQGTKSSKCVRQKVEGSSSHLVTFTV
LPLEIGLHNINFSLETWFGKEILVKTLRVVPEGVKRESYSGVTLDPRGIY
GTISRRKEFPYRIPLDLVPKTEIKRILSVKGLLVGEILSAVLSQEGINIL
THLPKGSAEAELMSVVPVFYVFHYLETGNHWNIFHSDPLIEKQKLKKKLK
EGMLSIMSYRNADYSYSVWKGGSASTWLTAFALRVLGQVNKYVEQNQNSI
CNSLLWLVENYQLDNGSFKENSQYQPIKLQGTLPVEARENSLYLTAFTVI
GIRKAFDICPLVKIDTALIKADNFLLENTLPAQSTFTLAISAYALSLGDK
THPQFRSIVSALKREALVKGNPPIYRFWKDNLQHKDSSVPNTGTARMVET
TAYALLTSLNLKDINYVNPVIKWLSEEQRYGGGFYSTQDTINAIEGLTEY
SLLVKQLRLSMDIDVSYKHKGALHNYKMTDKNFLGRPVEVLLNDDLIVST
GFGSGLATVHVTTVVHKTSTSEEVCSFYLKIDTQDIEASHYRGYGNSDYK
RIVACASYKPSREESSSGSSHAVMDISLPTGISANEEDLKALVEGVDQLF
TDYQIKDGHVILQLNSIPSSDFLCVRFRIFELFEVGFLSPATFTVYEYHR
PDKQCTMFYSTSNIKIQKVCEGAACKCVEADCGQMQEELDLTISAETRKQ
TACKPEIAYAYKVSITSITVENVFVKYKATLLDIYKTGEAVAEKDSEITF
IKKVTCTNAELVKGRQYLIMGKEALQIKYNFSFRYIYPLDSLTWIEYWPR
DTTCSSCQAFLANLDEFAEDIFLNGC

In some embodiments, an anti-C5 antibody as herein described (e.g., C5S2.2) competitively inhibits eculizumab from binding to human C5.

In some preferred embodiments, the anti-C5 antibody comprises a CDR1, CDR2 and/or CDR3 of the anti-C5 antibody of any one of SEQ ID Nos: 58 and 141 or comprises a CDR1, CDR2 and/or CDR3 at least 70% identical thereto (or comprising four or fewer amino acid substitutions relative to CDR1, CDR2, CDR3 of any one of SEQ ID Nos: 58 and 141).

The antibodies provided herein also include functional variants thereof. The term “functional variant” is meant to include those variants, which retain some or essentially all the ability of an antibody to selectively binding its antigen or ligand, such as any of the ligands mentioned herein below. Functional variants include any variant, which is at least 75% identical to antibodies provided herein, such as at least 80, 81, 82, 83, 84, 85, 86, 87, 88, 89, such as 90, 91, 92, 93, 94, 95, 96, such as 97, such as 98, such as 99, such as at least 99.5% identical to an antibody provided herein, such as any of those identified by SEQ ID NOs: 1-14 and 57-65. In a preferred embodiment, the antibody comprises the amino acid sequence set forth as SEQ ID NO: 14 or an amino acid sequence at least 75% identical thereto.

Functional variants include any variant antibody comprising one or more CDR(s), which is at least 75% identical to a CDR of an antibody provided herein, such as at least 80, 81, 82, 83, 84, 85, 86, 87, 88, 89, such as 90, 91, 92, 93, 94, 95, 96, such as 97, such as 98, such as 99, such as at least 99.5% identical to said CDR. The CDRs of the respective antibodies identified by SEQ ID NOs: 1-14 and 57-65 are indicated above as SEQ ID Nos: 15-56 and 66-92, respectively. Thus, antibodies are provided, which comprise one or more regions having at least 75%, such as at least 80, 81, 82, 83, 84, 85, 86, 87, 88, 89, such as 90, 91, 92, 93, 94, 95, 96, such as 97, such as 98, such as 99, such as at least 99.5% identity to one or more regions identified by any one of SEQ ID Nos: 15-56 and 66-92.

Amino acid substitutions include conservative amino acid substitutions, which refer to substitution of one amino acid with another amino acid residue having a side chain with similar properties. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Within the meaning of the term “conservative amino acid substitution” as applied herein, one amino acid may be substituted for another within the groups of amino acids indicated herein below:
i) Amino acids having polar side chains (Asp, Glu, Lys, Arg, His, Asn, Gin, Ser, Thr, Tyr, and Cys,)
ii) Amino acids having non-polar side chains (Gly, Ala, Val, Leu, lie, Phe, Trp, Pro, and Met)
iii) Amino acids having aliphatic side chains (Gly, Ala Val, Leu, lie)
iv) Amino acids having cyclic side chains (Phe, Tyr, Trp, His, Pro)
v) Amino acids having aromatic side chains (Phe, Tyr, Trp)
vi) Amino acids having acidic side chains (Asp, Glu)
vii) Amino acids having basic side chains (Lys, Arg, His)
viii) Amino acids having amide side chains (Asn, Gin)
ix) Amino acids having hydroxy side chains (Ser, Thr)
x) Amino acids having sulphur-containing side chains (Cys, Met),
xi) Neutral, weakly hydrophobic amino acids (Pro, Ala, Gly, Ser, Thr)
xii) Hydrophilic, acidic amino acids (Gin, Asn, Glu, Asp), and
xiii) Hydrophobic amino acids (Leu, lie, Val)

Functional variants of antibodies may in one preferred embodiment be a fragment of an antibody, preferably an antigen-binding fragment or a variable region. Examples of antibody fragments useful with the present invention include fragments of VHH and VNAR.

The antibodies may thus be obtained by immunization of any suitable organism, in particular camelids, sharks or the like. However, the antibodies could also be generated from a synthetic library with randomized or designed CDRs. In one embodiment, an antibody capable of specifically binding to an epitope of human complement factor C3, C3a and/or C3b provided. In another embodiment, an antibody capable of specifically binding to an epitope of human complement factor C5, C5a and/or C5b provided.

In one embodiment, the antibody is coupled to a histidine tag, such as a his6 tag at the N- or C-terminus of the polypeptide. In one preferred embodiment, the antibody is coupled to a non-native Strep or his6 tag (e.g., WSHPQFEKHHHHHH (SEQ ID NO:93) at the C-terminal. In another embodiment, the antibody is coupled to an Fc-fragment at the N-terminal. N-terminal additions are particularly preferred, and in a preferred embodiment, the antibody provided herein comprises an additional N-terminal region. The additional N-terminal region can be selected from any relevant additional moieties, depending on the contemplated application of the antibody and the desired functionalities to the final antibody product. Albumin may be added for increasing circulation time and protect the product from degradation. Other antigen binding fragments, antibodies or fragments thereof may be added for introducing a second affinity/binding specificity to the antibody product.

In some embodiments, an anti-C3 antibody as herein described forms a first domain in a fusion protein. In some aspects, the fusion protein is a heterodimeric fusion protein comprising a first domain comprising a polypeptide comprising an antibody as herein described and a second domain comprising an immunoglobulin Fc domain. In some aspects, the first domain and second domain are joined by a linker. In a preferred embodiment, the fusion protein is a heterodimeric fusion protein comprising the amino acid sequence of any one of SEQ ID Nos: 178-180 or an amino acid sequence at least 70% identical thereto. In other aspects, the fusion protein is a homodimeric fusion protein comprising a first domain comprising a polypeptide comprising an antibody as herein described and a second domain identical to the first domain. In some aspects, the first domain and second domain are joined by a linker, e.g., a poly-Glycine-Serine (G4S) linker, preferably a (G4S) 2 linker. In a preferred embodiment, the fusion protein is a homodimeric fusion protein comprising the amino acid sequence of SEQ ID NO:181 or 182 or an amino acid sequence at least 70% identical there.

In some embodiments, a bispecific/multispecific antibody is provided, e.g., a single peptide chain comprising two antigen-binding regions, which may be separated by a linker sequence, wherein one of the two antigen-binding regions comprises a VHH domain as herein described. In some aspects, a VHH domain as herein described is coupled to another VHH domain yielding a bispecific antibody, which consists of or comprises (i) a VHH domain as herein described and (ii) a VHH domain capable of specifically binding to an epitope of a second target.

The antibodies disclosed herein may in preferred embodiments comprise modifications, which improve the function of the antibody. For example, it is not always desirable to use non-human antibodies for human therapy, and accordingly an antibody provided herein may be a humanized antibody.

In some embodiments, the humanized antibody is a humanized form of the llama nanobodies described above and is selected from among the following amino acid sequences or amino acid sequences at least 75% identical thereto (the bolded amino acids are the humanized form amino acids that have been changed from the corresponding llama nanobody amino acid);

C3S1.1:
(SEQ ID NO: 94)
EVQLVESGGGLVQPGGSLRLSCAASGNISAPSLMGWYRQAPGKEREFVATITYGSITNY
ADSVKGRFTISRDNSKNTLYLQMNSLRAEDTAVYYCAANTLGIYNTYGDHHYWGQGT
LVTVSS
C3S1.2:
(SEQ ID NO: 95)
EVQLVESGGGLVQAGGSLRLSCAASGNISAPSLMGWYRQAPGKEREFVATITYGSITNY
VDSVKGRFTISRDNSKNTLYLQMNSLRPEDTAVYYCAANTLGIYNTYGDHHYWGQGT
LVTVSS
C3S1.3:
(SEQ ID NO: 96)
QVQLVESGGGLVQAGGSLRLSCAASGNISAPSLMGWYRQAPGKEREFVATITYGSITNY
VDSVKGRFTISRDNSKNTLYLQMNSLRPEDTAVYYCAANTLGIYNTYGDHHYWGQGT
LVTVSS
C3S3.1:
(SEQ ID NO: 97)
EVQLVESGGGLVQPGGSLRLSCAASGTIFSRNYMGWYRQAPGKERELVAGIGYGSSTN
YADSVKGRFTISRDNSKNTLYLQMNSLRAEDTAVYYCAAVPTYRPYYFYWGQGTLVT
VSS
C3S3.2:
(SEQ ID NO: 98)
EVQLVESGGGLVQAGGSLRLSCAASGTIFSRNYMGWYRQAPGKERELVAGIGYGSSTN
YADSVKGRFTISRDNSKNTLYLQMNSLRPEDTAVYYCAAVPTYRPYYFYWGQGTLVT
VSS
C3S3.3:
(SEQ ID NO: 99)
QVQLVESGGGLVQAGGSLRLSCAASGTIFSRNYMGWYRQAPGKERELVAGIGYGSSTN
YADSVKGRFTISRDNSKNTLYLQMNSLRPEDTAVYYCAAVPTYRPYYFYWGQGTLVT
VSS
C3S4.1:
(SEQ ID NO: 100)
EVQLVESGGGLVQPGGSLRLSCAASGTISGPGDMGWYRQAPGKERELVAGINYGAITY
YADSVKGRFTISRDNSKNTLYLQMNSLRAEDTAVYYCAADYETWAPSYEYFDYWGQG
TLVTVSS
C3S4.2:
(SEQ ID NO: 101)
EVQLVESGGGLVQAGGSLRLSCAASGTISGPGDMGWYRQAPGKERELVAGINYGAITY
YADSVKGRFTISRDNSKNTLYLQMNSLRPEDTAVYYCAADYETWAPSYEYFDYWGQG
TLVTVSS
C3S4.3:
(SEQ ID NO: 102)
QVQLVESGGGLVQAGGSLRLSCAASGTISGPGDMGWYRQAPGKERELVAGINYGAITY
YADSVKGRFTISRDNSKNTLYLQMNSLRPEDTAVYYCAADYETWAPSYEYEDYWGQG
TLVTVSS
C3S5.1:
(SEQ ID NO: 103)
EVQLVESGGGLVQPGGSLRLSCAASGTIFDDESMGWYRQAPGKEREFVASINYGSTTYY
ADSVKGRFTISRDNAKNSLYLQMNSLRAEDTAVYYCAVGWSTNYDWTLSYYYWGQG
TLVTVSS
C3S5.2:
(SEQ ID NO: 104)
EVQLVESGGGLVQAGGSLRLSCAASGTIFDDESMGWYRQAPGKEREFVASINYGSTTY
YADSVKGRFTISRDNAKNSLYLQMNSLRPEDTAVYYCAVGWSTNYDWTLSYYYWGQ
GTLVTVSS
C3S5.3:
(SEQ ID NO: 105)
QVQLVESGGGLVQAGGSLRLSCAASGTIFDDESMGWYRQAPGKEREFVASINYGSTTY
YADSVKGRFTISRDNAKNSLYLQMNSLRPEDTAVYYCAVGWSTNYDWTLSYYYWGQ
GTLVTVSS
C3S6.1:
(SEQ ID NO: 106)
EVQLVESGGGLVQPGGSLRLSCAASGNIFVGHGMGWYRQAPGKEREFVAAIGYGAITN
YADSVKGRFTISRDNSKNTLYLQMNSLRAEDTAVYYCAASSYYPPRYWYWGQGTLVT
VSS
C3S6.2:
(SEQ ID NO: 107)
EVQLVESGGGLVQAGGSLRLSCAASGNIFVGHGMGWYRQAPGKEREFVAAIGYGAITN
YADSVKGRFTISRDNSKNTLYLQMNSLRPEDTAVYYCAASSYYPPRYWYWGQGTLVT
VSS
C3S6.3:
(SEQ ID NO: 108)
QVQLVESGGGLVQAGGSLRLSCAASGNIFVGHGMGWYRQAPGKEREFVAAIGYGAITN
YADSVKGRFTISRDNSKNTLYLQMNSLRPEDTAVYYCAASSYYPPRYWYWGQGTLVT
VSS
C3S7.1:
(SEQ ID NO: 109)
EVQLVESGGGLVQPGGSLRLSCAASGYIFTGVDMGWYRQAPGKEREFVAAISYGASTY
YADSVKGRFTISRDNSKNTLYLQMNSLRAEDTAVYYCAASLSNTYLLDLFFRYWGQGT
LVTVSS
C3S7.2:
(SEQ ID NO: 110)
EVQLVESGGGLVQAGGSLRLSCAASGYIFTGVDMGWYRQAPGKEREFVAAISYGASTY
YADSVKGRFTISRDNSKNTLYLQMNSLRPEDTAVYYCAASLSNTYLLDLFFRYWGQGT
LVTVSS
C3S7.3:
(SEQ ID NO: 111)
QVQLVESGGGLVQAGGSLRLSCAASGYIFTGVDMGWYRQAPGKEREFVAAISYGASTY
YADSVKGRFTISRDNSKNTLYLQMNSLRPEDTAVYYCAASLSNTYLLDLFFRYWGQGT
LVTVSS
C3S8.1:
(SEQ ID NO: 112)
EVQLVESGGGLVQPGGSLRLSCAASGTIFPTDRMGWYRQAPGKEREFVATIDLGGTTN
YADSVKGRFTISRDNSKNTLYLQMNSLRAEDTAVYYCAAAWEGPSRYGRHRYYSHRY
WGQGTLVTVSS
C3S8.2:
(SEQ ID NO: 113)
EVQLVESGGGLVQAGGSLRLSCAASGTIFPTDRMGWYRQAPGKEREFVATIDLGGTTN
YADSVKGRFTISRDNSKNTLYLQMNSLRAEDTAVYYCAAAWEGPSRYGRHRYYSHRY
WGQGTLVTVSS
C3S8.3:
(SEQ ID NO: 114)
QVQLVESGGGLVQAGGSLRLSCAASGTIFPTDRMGWYRQAPGKEREFVATIDLGGTIN
YADSVKGRFTISRDNSKNTLYLQMNSLRPEDTAVYYCAAAWEGPSRYGRHRYYSHRY
WGQGTLVTVSS
C3S9.1:
(SEQ ID NO: 115)
EVQLVESGGGLVQPGGSLRLSCAASGYIFVYSTMGWYRQAPGKEREFVASINYGSITYY
ADSVKGRFTISRDNSKNTLYLQMNSLRAEDTAVYYCAVTDKSRYWLTYWGQGTLVTV
SS
C3S9.2:
(SEQ ID NO: 116)
EVQLVESGGGLVQAGGSLRLSCAASGYIFVYSTMGWYRQAPGKEREFVASINYGSITYY
ADSVKGRFTISRDNSKNTLYLQMNSLRPEDTAVYYCAVIDKSRYWLTYWGQGTLVTV
SS
C389.3:
(SEQ ID NO: 117)
QVQLVESGGGLVQAGGSLRLSCAASGYIFVYSTMGWYRQAPGKEREFVASINYGSITYY
ADSVKGRFTISRDNSKNTLYLQMNSLRPEDTAVYYCAVTDKSRYWLTYWGQGTLVTV
SS
C3S10.1:
(SEQ ID NO: 118)
EVQLVESGGGLVQPGGSLRLSCAASGYISGGSIMGWYRQAPGKEREFVAGINQGSNTY
YADSVKGRFTISRDNSKNTLYLOMNSLRAEDTAVYYCAVGGRSYYYHHYWGQGTLVT
VSS
C3S10.2:
(SEQ ID NO: 119)
EVQLVESGGGLVQAGGSLRLSCAASGYISGGSIMGWYRQAPGKEREFVAGINQGSNTY
YADSVKGRFTISRDNSKNTLYLQMNSLRPEDTAVYYCAVGGRSYYYHHYWGQGTLVT
VSS
C3S10.3:
(SEQ ID NO: 120)
QVQLVESGGGLVQAGGSLRLSCAASGYISGGSIMGWYRQAPGKEREFVAGINQGSNTY
YADSVKGRFTISRDNSKNTLYLQMNSLRPEDTAVYYCAVGGRSYYYHHYWGQGTLVT
VSS
C313.1:
(SEQ ID NO: 121)
EVQLLESGGGLVQPGGSLRLSCAASGFTLDNYAIAWFRQAPGKEREGVSCISSSRGSTA
YEDSVKGRFTISRDNSKNTLYLQMNSLRAEDTAVYYCAARARGMCREFDYDYWGQGT
LVTVSS
C313.2:
(SEQ ID NO: 122)
EVQLLESGGGLVQPGGSLRLSCAASGFTLDNYAIAWFRQAPGKEREGVSCISSSRGSTA
YEDSVKGRFTISRDNSKNTLYLQMNSLRPEDTAVYYCAARARGMCREFDYDYWGQGT
LVTVSS
C313.3:
(SEQ ID NO: 123)
EVQLLESGGGLVQPGGSLRLSCAASGFTLDNYAIAWFRQAPGKEREGVSCISSSRGSTA
YEDSVKGRFTISRDNSKNTLYLQMNSLKPEDTAVYYCAARARGMCREFDYDYWGQGT
LVTVSS
C3I4.1:
(SEQ ID NO: 124)
EVQLLESGGGLVQPGGSLRLSCATSGETLDYYAIGWFRQAPGKEREGVSCITGPDGSTH
YADSVKGRFTISRDNSKNTLYLQMNSLRAEDTAVYYCAATAGLYGCGYGMDHWGKG
TLVTVSS
C3I4.2:
(SEQ ID NO: 125)
EVQLLESGGGLVQPGGSLRLSCATSGFTLDYYAIGWFRQAPGKEREGVSCITGPDGSTH
YADSVKGRFTISRDNSKNTLYLQMNSLRPEDTAVYYCAATAGLYGCGYGMDHWGKG
TLVTVSS
C3I4.3:
(SEQ ID NO: 126)
EVQLLESGGGLVQPGGSLRLSCATSGFTLDYYAIGWFRQAPGKEREGVSCITGPDGSTH
YADSVKGRFTISRDNSKNTLYLQMNSLKPEDTAVYYCAATAGLYGCGYGMDHWGKG
TLVTVSS
C318.1:
(SEQ ID NO: 127)
EVQLLESGGGLVQPGGSLRLSCATSGFSFGVYDMSWVRQASGKGLEWVSGIDSGSNTA
YADSVKGRFTISRDNSKNTLYLQMNSLRAEDTAVYYCALGRETDYIPVTDFRTQGTLV
TVSS
C318.2:
(SEQ ID NO: 128)
EVQLLESGGGLVQPGGSLRLSCATSGFSFGVYDMSWVRQASGKGLEWVSGIDSGSNTA
YADSVKGRFTISRDNSKNTLYLQMNNLRPEDTAVYYCALGRETDYIPVTDFRTQGTLV
TVSS
C318.3:
(SEQ ID NO: 129)
EVQLLESGGGLVQPGGSLRLSCATSGFSFGVYDMSWVRQASGKGLEWVSGIDSGSNTA
YADSVKVRFTISRDNSKNTLYLQMNNLKPEDTAVYYCALGRETDYIPVTDFRTQGTLVT
VSS
C3122.1:
(SEQ ID NO: 130)
EVQLLESGGGLVQPGGSLRLSCTASGFTFGVYAMSWVRQAPGKGLEWVSSIDSGVNTA
YADSVKGRFTISRDNSKNTLYLQMNSLRAEDTAVYHCALGRESDYEPVTGSWGQGTL
VTVSS
C3122.2:
(SEQ ID NO: 131)
EVQLLESGGGLVQPGGSLRLSCTASGFTFGVYAMSWVRQAPGKGLEWVSSIDSGVNTA
YADSVKGRFTISRDNSINTLYLQMNSLRPEDTAVYHCALGRESDYEPVTGSWGQGTLV
TVSS
C3122.3:
(SEQ ID NO: 132)
EVQLLESGGGLVQPGGSLRLSCTASGFTFGVYAMSWVRQAPGKGLEWVSSIDSGVNTA
YADSVKGRFTISRDNSINTLFLQMNSLKPEDTAVYHCALGRESDYEPVTGSWGQGTLVT
VSS
C3N10.1;
(SEQ ID NO: 133)
QVQLVESGGGLVQPGGSLRLACAASGLTFSPYAMGWFRQAPGKEREFVAGIRWISGNP
YYADSVKGRFTISRDNSKNTLYLQMNSLRAEDTAVYSCAAAPLTRSYDYWGQGTQVT
VSS
C3N10.2:
(SEQ ID NO: 134)
EVQLVESGGGLVQPGGSLRLACAASGLTFSPYAMGWFRQAPGKEREFVAGIRWISGNP
YYADSVKGRFTISRDNSKNTLYLQMNSLRAEDTAVYSCAAAPLTRSYDYWGQGTLVT
VSS
C3N10.3:
(SEQ ID NO: 135)
EVQLVESGGGLVQAGGSLRLACAASGLTFSPYAMGWFRQAPGKEREFVAGIRWISGNP
YYADSVKGRFTISRDNSKNTLYLQMNSLRPEDTAVYSCAAAPLTRSYDYWGQGTLVTV
SS
C3N10.4:
(SEQ ID NO: 136)
EVQLVESGGGLVQAGGSLRLACAASGLTFSPYAMGWFRQAPGKEREFVAGIRWISGNP
YYADSVKGRFTISRDNSKNTLYLQMNSLKPEDTAVYSCAAAPLTRSYDYWGQGTLVTV
SS
C5S1.1:
(SEQ ID NO: 137)
EVQLVESGGGLVQPGGSLRLSCAASGYISYDDDMGWYRQAPGKEREFVAAIDVGGSTY
YADSVKGRFTISRDNSKNTLYLQMNSLRAEDTAVYYCAVRDGYYWDHDYWGQGTLV
TVSS
C5S1.2:
(SEQ ID NO: 138)
EVQLVESGGGLVQAGGSLRLSCAASGYISYDDDMGWYRQAPGKEREFVAAIDVGGST
YYADSVKGRFTISRDNSKNTLYLQMNSLRPEDTAVYYCAVRDGYYWDHDYWGQGTL
VTVSS
C5S1.3:
(SEQ ID NO: 139)
QVQLVESGGGLVQAGGSLRLSCAASGYISYDDDMGWYRQAPGKEREFVAAIDVGGST
YYADSVKGRFTISRDNSKNTLYLQMNSLKPEDTAVYYCAVRDGYYWDHDYWGQGTL
VTVSS
C5S2.1:
(SEQ ID NO: 140)
EVQLVESGGGLVQPGGSLRLSCAASGNIFHQSDMGWYRQAPGKEREFVAAIDYGTNTY
YADSVKGRFTISRDNSKNTLYLQMNSLRAEDTAVYYCAARDTSYWDHVYWGQGTLV
TVSS
C5S2.2:
(SEQ ID NO: 141)
EVQLVESGGGLVQAGGSLRLSCAASGNIFHQSDMGWYRQAPGKEREFVAAIDYGTNTY
YADSVKGRFTISRDNSKNTLYLQMNSLRPEDTAVYYCAARDTSYWDHVYWGQGTLVT
VSS
C5S2.3:
(SEQ ID NO: 142)
QVQLVESGGGLVQAGGSLRLSCAASGNIFHQSDMGWYRQAPGKEREFVAAIDYGINTY
YADSVKGRFTISRDNSKNTLYLQMNSLKPEDTAVYYCAARDTSYWDHVYWGQGTLVT
VSS
C5S3.1:
(SEQ ID NO: 143)
EVQLVESGGGLVQPGGSLRLSCAASGSISFDTDMGWYRQAPGKEREFVATIDGGTSTYY
ADSVKGRFTISRDNSKNTLYLQMNSLRAEDTAVYYCAAEAGYFSLLGPYFRYWGQGTL
VTVSS
C5S3.2:
(SEQ ID NO: 144)
EVQLVESGGGLVQAGGSLRLSCAASGSISFDTDMGWYRQAPGKEREFVATIDGGTSTY
YADSVKGRFTISRDNSKNTLYLQMNSLRPEDTAVYYCAAEAGYFSLLGPYFRYWGQGT
LVTVSS
C5S3.3:
(SEQ ID NO: 145)
QVQLVESGGGLVQAGGSLRLSCAASGSISFDTDMGWYRQAPGKEREFVATIDGGTSTY
YADSVKGRFTISRDNSKNTLYLQMNSLKPEDTAVYYCAAEAGYFSLLGPYFRYWGQGT
LVTVSS
CSS4.1:
(SEQ ID NO: 146)
EVQLVESGGGLVQPGGSLRLSCAASGYIFAATDMGWYRQAPGKEREFVATIDYGANTN
YADSVKGRFTIGRDNSKNTLYLQMNSLRAEDTAVYYCAARDNNYYDLVYWGQGTLV
TVSS
C5S4.2:
(SEQ ID NO: 147)
EVQLVESGGGLVQAGGSLRLSCAASGYIFAATDMGWYRQAPGKEREFVATIDYGANTN
YADSVKGRFTIGRDNSKNTLYLQMNSLRPEDTAVYYCAARDNNYYDLVYWGQGTLVT
VSS
C5S4.3:
(SEQ ID NO: 148)
QVQLVESGGGLVQAGGSLRLSCAASGYIFAATDMGWYRQAPGKEREFVATIDYGANTN
YADSVKGRFTIGRDNSKNTLYLQMNSLKPEDTAVYYCAARDNNYYDLVYWGQGTLVT
VSS
C585.1:
(SEQ ID NO: 149)
EVQLVESGGGLVQPGGSLRLSCAASGTISPYEGMGWYRQAPGKERELVAAIDGGGITY
YADSVKGRFTISRDNSKNTLYLQMNSLRAEDTAVYYCAAGIWTLSYYDYWGQGTLVT
VSS
C5S5.2:
(SEQ ID NO: 150)
EVQLVESGGGLVQAGGSLRLSCAASGTISPYEGMGWYRQAPGKERELVAAIDGGGITY
YADSVKGRFTISRDNSKNTLYLQMNSLRPEDTAVYYCAAGIWTLSYYDYWGQGTLVT
VSS
C5S5.3:
(SEQ ID NO: 151)
QVQLVESGGGLVQAGGSLRLSCAASGTISPYEGMGWYRQAPGKERELVAAIDGGGITY
YADSVKGRFTISRDNSKNTLYLQMNSLKPEDTAVYYCAAGIWTLSYYDYWGQGTLVT
VSS
C5S6.1:
(SEQ ID NO: 152)
EVQLVESGGGLVQPGGSLRLSCAASGTIFYSYKMGWYRQAPGKEREFVAGITYGGSTY
YADSVKGRFTISRDNSKNTLYLQMNSLRAEDTAVYYCAARAYYVSVYDTALYYWGQ
GTLVTVSS
C5S6.2:
(SEQ ID NO: 153)
EVQLVESGGGLVQAGGSLRLSCAASGTIFYSYKMGWYRQAPGKEREFVAGITYGGSTY
YADSVKGRFTISRDNSKNTLYLQMNSLRPEDTAVYYCAARAYYVSVYDTALYYWGQG
TLVTVSS
C5S6.3:
(SEQ ID NO: 154)
QVQLVESGGGLVQAGGSLRLSCAASGTIFYSYKMGWYRQAPGKEREFVAGITYGGSTY
YADSVKGRFTISRDNSKNTLYLQMNSLKPEDTAVYYCAARAYYVSVYDTALYYWGQG
TLVTVSS
C5S7.1:
(SEQ ID NO: 155)
EVQLVESGGGLVQPGGSLRLSCAASGYIFYDDDMGWYRQAPGKEREFVATIDVGGNTN
YADSVKGRFTIGRDNSKNTLYLQMNSLRAEDTAVYYCAVRDNNYYDHVYWGQGTLV
TVSS
C5S7.2:
(SEQ ID NO: 156)
EVQLVESGGGLVQAGGSLRLSCAASGYIFYDDDMGWYRQAPGKEREFVATIDVGGNT
NYADSVKGRFTIGRDNSKNTLYLQMNSLRPEDTAVYYCAVRDNNYYDHVYWGQGTL
VTVSS
C5S7.3:
(SEQ ID NO: 157)
QVQLVESGGGLVQAGGSLRLSCAASGYIFYDDDMGWYRQAPGKEREFVATIDVGGNT
NYADSVKGRFTIGRDNSKNTLYLQMNSLKPEDTAVYYCAVRQNNYYDHVYWGQGTL
VTVSS
C5S8.1:
(SEQ ID NO: 158)
EVQLVESGGGLVQPGGSLRLSCAASGNIFDEWDMGWYRQAPGKEREFVAAIDDGAST
YYADSVKGRFTISRDNSKNTLYLQMNSLRAEDTAVYYCAARQTYAWDYRYWGQGTL
VTVSS
C588.2:
(SEQ ID NO: 159)
EVQLVESGGGLVQAGGSLRLSCAASGNIFDEWDMGWYRQAPGKEREFVAAIDDGAST
YYADSVKGRFTISRDNSKNTLYLQMNSLRPEDTAVYYCAARQTYAWDYRYWGQGTL
VTVSS
CSS8.3:
(SEQ ID NO: 160)
QVQLVESGGGLVQAGGSLRLSCAASGNIFDEWDMGWYRQAPGKEREFVAAIDDGAST
YYADSVKGRFTISRDNSKNTLYLQMNSLKPEDTAVYYCAARQTYAWDYRYWGQGTL
VTVSS
C5S12.1:
(SEQ ID NO: 161)
EVQLVESGGGLVQPGGSLRLSCAASGYIFPSDDMGWYRQAPGKEREFVAAINDGASTY
YADSVKGRFTISRDNSKNTLYLQMNSLRAEDTAVYYCAVQLNYYWDYVYWGQGTLV
TVSS
C5S12.2:
(SEQ ID NO: 162)
EVQLVESGGGLVQAGGSLRLSCAASGYIFPSDDMGWYRQAPGKEREFVAAINDGAST
YYADSVKGRFTISRDNSKNTLYLQMNSLRPEDTAVYYCAVQLNYYWDYVYW
GQGTLVTVSS
C5S12.3:
(SEQ ID NO: 163)
QVQLVESGGGLVQAGGSLRLSCAASGYIFPSDDMGWYRQAPGKEREFVAAINDGASTY
YADSVKGRFTISRDNSKNTLYLQMNSLKPEDTAVYYCAVQLNYYWDYVYWGQGTLV
TVSS

In some preferred embodiments, the antibody comprises the sequence of any one of SEQ ID Nos: 94-163 or comprises an amino acid sequence at least 75%, such as at least 80%, 81%, 82%, 83%, 84%, 85%, 86%, 87%, 88%, 89%, 90%, 91%, 92%, 93%, 94%, 95%, 96%, 97%, 98%, 99%, or at least 99.5% identical thereto and preferably comprises the bolded amino acids.

In addition to the antibodies provided herein, a nucleic acid comprising a nucleotide sequence encoding an antibody as herein described is provided. In related embodiments, the nucleic acid encoding the antibody as herein described forms part of an expression vector in which the nucleotide sequence encoding the antibody is operably linked to a suitable promoter.

In one embodiment, the RNA and/or cDNA coding sequences are designed for optimal expression in human cells. Codon-optimized coding regions can be designed by various different methods. This optimization may be performed using methods which are available on-line, published methods, or a company which provides codon optimizing services. One codon optimizing method is described, e.g., in WO 2015/012924 A2, which is incorporated by reference herein. Briefly, the nucleic acid sequence encoding the product is modified with synonymous codon sequences. Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide.

In some embodiments, a pharmaceutical composition is provided comprising a pharmaceutically acceptable carrier and a nucleic acid comprising a nucleotide sequence encoding an antibody as herein described, said nucleotide sequence operably linked to a promoter. In other embodiments, a pharmaceutical composition is provided comprising an antibody as herein described and a pharmaceutically acceptable carrier.

Recombinant AAV Virions Encoding Anti-C3 and/or Anti-C5 Antibodies

The novel rAAV virions described herein comprise (i) a capsid as herein described and (ii) a heterologous nucleic acid encoding an anti-C3 and/or anti-C5 antibody as herein described, preferably wherein the nucleotide sequence encoding the anti-C3 and/or anti-C5 antibody is operably linked to an expression control sequence. Typically, the heterologous nucleic acid includes an AAV genome with the rep and cap genes deleted and/or replaced by the antibody sequence and its associated expression control sequences. The antibody sequence is typically inserted adjacent to one or two (i.e., is flanked by) AAV TRs or TR elements adequate for viral replication (Xiao et al., 1997, J. Virol. 71(2): 941-948), in place of the nucleic acid encoding viral rep and cap proteins. Other regulatory sequences suitable for use in facilitating tissue-specific expression of the antibody y gene sequence in the target cell (e.g., a retinal cell) may also be included.

In some aspects, the anti-C3 antibody encoded by the heterologous nucleic acid of the rAAV is able to prevent binding of C3 to the C3 convertases of the alternative pathway and the classical pathway which in turn inhibits C3 cleavage.

Heterologous Nucleic Acid Component of the rAAV

In some aspects, the rAAV virion comprises a heterologous nucleic acid comprising (a) an AAV2 terminal repeat (b) a transcription control sequence (c) a nucleotide sequence encoding an anti-C3 antibody as herein described (d) a polyadenylation sequence and (e) an AAV2 terminal repeat.

In other aspects, the rAAV virion comprises a heterologous nucleic acid comprising (a) an AAV2 terminal repeat (b) a transcription control sequence (c) a nucleotide sequence encoding an anti-C5 antibody as herein described (d) a polyadenylation sequence and (e) an AAV2 terminal repeat.

The inverted terminal repeats (ITR(s)) selected for use in the rAAV virion are preferably AAV sequences, with serotypes 1, 2, 3, 4, 5 and 6 being preferred. The ITRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both ITR sequences are from AAV2.

In some aspects, the heterologous nucleic encapsulated by the rAAV virion comprises a 5′ ITR with the following sequence:

(SEQ ID NO: 164)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCT

In related aspects, the heterologous nucleic acid encapsulated by the rAAV virion comprises a 3′ ITR with the following sequence:

(SEQ ID NO: 165)
AGGAACCCCTAGTGATGGAGTTGGCCACTCCCTCTCTGCGCGCTCGCTCG
CTCACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAA

The antibody gene encoded by the rAAV is preferably operatively linked to at least one transcription control sequence, preferably a transcription control sequence that is heterologous to the nucleic acid. In some aspects, the transcription control sequence comprises a cell- or tissue-specific promoter that results in cell-specific expression of the nucleic acid e.g. in photoreceptor cells, such as human rod photoreceptor-specific human G-protein coupled receptor rhodopsin kinase 1 (hGRK) promoter or a human interphotoreceptor retinoid-binding protein (IRBP) promoter. In other aspects, the transcription control sequence comprises a constitutive promoter that results in similar expression level of the nucleic acid in many cell types. Suitable constitutive promoters include CAG promoter comprising (C) cytomegalovirus (CMV) immediate-early enhancer element, (A) first exon and the first intron of chicken beta-actin gene and (G) splice acceptor of the rabbit beta-globin gene (see Miyazaki et al. (1989) Gene 79(2): 269-277), cytomegalovirus promoter (CMV) (Stinski et al, (1985) Journal of Virology 55(2): 431-441), human elongation factor 1α promoter (EF1α) (Kim et al. (1990) Gene 91(2): 217-223), human phosphoglycerate kinase promoter (PGK) (Singer-Sam et al. (1984) Gene 32(3): 409-417), mitochondrial heavy-strand promoter (Loderio et al. (2012) PNAS 109(17): 6513-6518), and ubiquitin promoter (Wulff et al. (1990) FEBS Letters 261:101-105).

In a preferred aspect, the antibody gene encoded by the rAAV is operably linked to a CAG promoter. In a particularly preferred embodiment, the CAG promoter comprises the sequence of SEQ ID NO:166 or comprises a sequence at least 95%, at least 96%, at least 97%, at least 98% or at least 99% identical thereto:

(SEQ ID NO: 166)
ACTAGTTATTAATAGTAATCAATTACGGGGTCATTAGTTCATAGCCCATA
TATGGAGTTCCGCGTTACATAACTTACGGTAAATGGCCCGCCTGGCTGAC
CGCCCAACGACCCCCGCCCATTGACGTCAATAATGACGTATGTTCCCATA
GTAACGCCAATAGGGACTTTCCATTGACGTCAATGGGTGGAGTATTTACG
GTAAACTGCCCACTTGGCAGTACATCAAGTGTATCATATGCCAAGTACGC
CCCCTATTGACGTCAATGACGGTAAATGGCCCGCCTGGCATTATGCCCAG
TACATGACCTTATGGGACTTTCCTACTTGGCAGTACATCTACGTATTAGT
CATCGCTATTACCATGGTCGAGGTGAGCCCCACGTTCTGCTTCACTCTCC
CCATCTCCCCCCCCTCCCCACCCCCAATTTTGTATTTATTTATTTTTTAA
TTATTTTGTGCAGCGATGGGGGCGGGGGGGGGGGGGGGGCGCGCGCCAGG
CGGGGGGGGCGGGGCGAGGGGCGGGGCGGGGCGAGGCGGAGAGGTGCGGC
GGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGGAGT
CGCTGCGACGCTGCCTTCGCCCCGTGCCCCGCTCCGCCGCCGCCTCGCGC
CGCCCGCCCCGGCTCTGACTGACCGCGTTACTCCCACAGGTGAGCGGGCG
GGACGGCCCTTCTCCTCCGGGCTGTAATTAGCGCTTGGTTTAATGACGGC
TTGTTTCTTTTCTGTGGCTGCGTGAAAGCCTTGAGGGGCTCCGGGAGGGC
CCTTTGTGCGGGGGGAGCGGCTCGGGGGGTGCGTGCGTGTGTGTGTGCGT
GGGGAGCGCCGCGTGCGGCTCCGCGCTGCCCGGCGGCTGTGAGCGCTGCG
GGCGCGGCGCGGGGCTTTGTGCGCTCCGCAGTGTGCGCGAGGGGAGCGCG
GCCGGGGGCGGTGCCCCGCGGTGCGGGGGGGGCTGCGAGGGGAACAAAGG
CTGCGTGCGGGGTGTGTGCGTGGGGGGGTGAGCAGGGGGTGTGGGCGCGT
CGGTCGGGCTGCAACCCCCCCTGCACCCCCCTCCCCGAGTTGCTGAGCAC
GGCCCGGCTTCGGGTGCGGGGCTCCGTACGGGGCGTGGCGCGGGGCTCGC
CGTGCCGGGCGGGGGGTGGCGGCAGGTGGGGGTGCCGGGCGGGGCGGGGC
CGCCTCGGGCCGGGGAGGGCTCGGGGGAGGGGCGCGGCGGCCCCCGGAGC
GCCGGCGGCTGTCGAGGCGCGGCGAGCCGCAGCCATTGCCTTTTATGGTA
ATCGTGCGAGAGGGCGCAGGGACTTCCTTTGTCCCAAATCTGTGCGGAGC
CGAAATCTGGGAGGCGCCGCCGCACCCCCTCTAGCGGGCGCGGGGCGAAG
CGGTGCGGCGCCGGCAGGAAGGAAATGGGGGGGGAGGGCCTTCGTGCGTC
GCCGCGCCGCCGTCCCCTTCTCCCTCTCCAGCCTCGGGGCTGTCCGCGGG
GGGACGGCTGCCTTCGGGGGGGACGGGGCAGGGGGGGGTTCGGCTTCTGG
CGTGTGACCGGCGGCTCTAGAGCCTCTGCTAACCATGTTCATGCCTTCTT
CTTTTTCCTACAG

In some aspects, the heterologous nucleic acid encapsulated by the rAAV virion comprises a SV40 polyadenylation sequence the following sequence:

(SEQ ID NO: 167)
GGGGATCCAGACATGATAAGATACATTGATGAGTTTGGACAAACCACAAC
TAGAATGCAGTGAAAAAAATGCTTTATTTGTGAAATTTGTGATGCTATTG
CTTTATTTGTAACCATTATAAGCTGCAATAAACAAGTTAACAACAACAAT
TGCATTCATTTTATGTTTCAGGTTCAGGGGGAGGTGTGGGAGGTTTTTTA
AAGCAAGTAAAACCTCTACAAATGTGGTATGGCTGATTATGATCA

Capsid Component of the rAAV

The variant AAV capsid of the rAAV—encapsulating the heterologous nucleic acid encoding an antibody or functional variant thereof—comprises a variant AAV capsid protein comprising an insertion of from about 7 to about 20 amino acids (a “heterologous peptide” or “peptide insertion”) in the GH-loop of a parental AAV capsid protein, wherein the peptide comprises the amino acid sequence ISDQTKH (SEQ ID NO:168). Preferably, the variant capsid protein, when present in an AAV virion, confers increased infectivity of a retinal cell compared to the infectivity of a retinal cell by an AAV virion comprising the corresponding parental capsid protein.

By the “GH loop,” or loop IV, of the AAV capsid protein it is meant the solvent-accessible portion referred to in the art as the GH loop, or loop IV, of AAV capsid protein. For the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. Thus, for example, the insertion site can be within about amino acids 570-611 of AAV2 VP1.

In some embodiments, the peptide insertion has from 1 to 3 spacer amino acids (Y₁-Y₃) at the amino and/or carboxyl terminus of the amino acid sequence ISDQTKH (SEQ ID NO: 168). Exemplary spacer amino acids include, without limitation, leucine (L), alanine (A), glycine (G), serine(S), threonine (T), and proline (P). In certain embodiments, a peptide insertion comprises 2 spacer amino acids at the N-terminus and 2 spacer amino acids at the C-terminus. In other embodiments, a peptide insertion comprises 2 spacer amino acids at the N-terminus and 1 spacer amino acids at the C-terminus. In preferred embodiments, the peptide insertion comprises or consists of the amino acid sequence LAISDQTKHA (SEQ ID NO:169).

In some aspects, the variant AAV capsid protein comprises a peptide insertion comprising the amino acid sequence ISDQTKH (SEQ ID NO:168) and further comprises one or more amino acid substitutions relative to a corresponding parental AAV capsid protein. Representative examples of amino acid substitutions may be found at e.g., col. 26, lines 40-65 of U.S. Pat. No. 11,576,983, the entire contents of which are incorporated herein by reference.

In some preferred embodiments, the variant AAV capsid protein comprises a peptide insertion comprising the amino acid sequence ISDQTKH (SEQ ID NO:168) and further comprises a P34A amino acid substitution relative to VP1 capsid of AAV2 or the corresponding substitution in another AAV serotype.

In other aspects, the variant capsid protein may comprise one or more features disclosed in U.S. Pat. No. 11,576,983, in particular, one or more features disclosed at column 26, line 66 to column 29, line 50 of U.S. Pat. No. 11,576,983.

In a particularly preferred embodiment, the variant capsid protein comprises the following amino acid sequence or comprises an amino acid sequence at least 80%, at least 90%, least 95%, at least 98%, or at least 99% identical to the following amino acid sequence;

(SEQ ID NO: 170)
MAADGYLPDWLEDTLSEGIRQWWKLKPGPPPPKAAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWORLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNLAISDQTKHARQA
ATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKEN
SKRWNPEIQYTSNYNKSVNVDFTVDINGVYSEPRPIGTRYLTRNL

The variant AAV capsid protein of SEQ ID NO: 170 contains the following modifications relative to native AAV2 capsid: (i) a proline (P) to alanine (A) mutation at amino acid position 34, which is located inside the assembled capsid (VP1 protein only), and (ii) an insertion of 10 amino acids (leucine-alanine-isoleucine-serine-aspartic acid-glutamine-threonine-lysine-histidine-alanine/LAISDQTKHA (SEQ ID NO:169)) at amino acid position 588, which is present in VP1, VP2, and VP3. In some embodiments, the capsid comprises a variant capsid protein comprising a sequence at least 90%, at least 95%, at least 98%, at least 99% identical to SEQ ID NO:170 and comprising a P34A substitution and an LAISDQTKHA (SEQ ID NO:169) peptide insertion at amino acid position 588.

Also provided herein are packaging cells, which are encompassed by “host cells,” which may be cultured to produce packaged viral vectors of the invention. The packaging cells of the invention generally include cells with heterologous (1) viral vector function(s), (2) packaging function(s), and (3) helper function(s). Each of these component functions is discussed in the ensuing sections.

Initially, the vectors can be made by several methods known to skilled artisans (see, e.g., WO 2013/063379), A preferred method is described in Grieger, et al. 2015, Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using the triple transfection method (e.g., WO 96/40240), the suspension HEK293 cell line generates greater than 10⁵ vector genome containing particles (vg)/cell or greater than 10¹⁴ vg/L of cell culture when harvested 48 hours post-transfection. More specifically, triple transfection refers to the fact that the packaging cell is transfected with three plasmids: one plasmid encodes the AAV rep and cap genes, another plasmid encodes various helper functions (e.g., adenovirus or HSV proteins such as E1a, E1b, E2a, E4, and VA RNA, and another plasmid encodes the transgene and its various control elements (e.g., modified GLA gene and CAG promoter).

To achieve the desired yields, a number of variables are optimized such as selection of a compatible serum-free suspension media that supports both growth and transfection, selection of a transfection reagent, transfection conditions and cell density. A universal purification strategy, based on ion exchange chromatography methods, was also developed that resulted in high purity vector preps of AAV serotypes 1-6, 8, 9 and various chimeric capsids. This user-friendly process can be completed within one week, results in high full to empty particle ratios (>90% full particles), provides post-purification yields (>1×10\hat{ }13 vg/L) and purity suitable for clinical applications and is universal with respect to all serotypes and chimeric particles. This scalable manufacturing technology has been utilized to manufacture GMP Phase I clinical AAV vectors for retinal neovascularization (AAV2), Hemophilia B (scAAV8), Giant Axonal Neuropathy (scAAV9) and Retinitis Pigmentosa (AAV2), which have been administered into patients. In addition, a minimum of a 5-fold increase in overall vector production by implementing a perfusion method that entails harvesting rAAV from the culture media at numerous time-points post-transfection.

The packaging cells include viral vector functions, along with packaging and vector functions. The viral vector functions typically include a portion of a parvovirus genome, such as an AAV genome, with rep and cap deleted and replaced by the modified GLA sequence and its associated expression control sequences. The viral vector functions include sufficient expression control sequences to result in replication of the viral vector for packaging. Typically, the viral vector includes a portion of a parvovirus genome, such as an AAV genome with rep and cap deleted and replaced by the transgene and its associated expression control sequences. The transgene is typically flanked by two AAV TRs, in place of the deleted viral rep and cap ORFs. Appropriate expression control sequences are included, such as a tissue-specific promoter and other regulatory sequences suitable for use in facilitating tissue-specific expression of the transgene in the target cell. The transgene is typically a nucleic acid sequence that can be expressed to produce a therapeutic polypeptide or a marker polypeptide.

The terminal repeats (TR(s)) (resolvable and non-resolvable) selected for use in the viral vectors are preferably AAV sequences, with serotypes 1, 2, 3, 4, 5 and 6 being preferred. Resolvable AAV TRs need not have a wild-type TR sequence (e.g., a wild-type sequence may be altered by insertion, deletion, truncation or missense mutations), as long as the TR mediates the desired functions, e.g., virus packaging, integration, and/or provirus rescue, and the like. The TRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both TR sequences are from AAV2.

The packaging functions include variant capsid components as described above.

The packaged viral vector includes an anti-C3 antibody transgene and expression control sequences flanked by TR elements, referred to herein as the “transgene” or “transgene expression cassette,” sufficient to result in packaging of the vector DNA and subsequent expression of the gene sequence in the transduced cell. The viral vector functions may, for example, be supplied to the cell as a component of a plasmid or an amplicon. The viral vector functions may exist extrachromosomally within the cell line and/or may be integrated into the cell's chromosomal DNA.

Any method of introducing the nucleotide sequence carrying the viral vector functions into a cellular host for replication and packaging may be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

The packaging functions include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions may be supplied together or separately to the packaging cell using a genetic construct such as a plasmid or an amplicon, a Baculovirus, or HSV helper construct. The packaging functions may exist extrachromosomally within the packaging cell, but are preferably integrated into the cell's chromosomal DNA. Examples include genes encoding AAV Rep and Cap proteins.

The helper functions include helper virus elements needed for establishing active infection of the packaging cell, which is required to initiate packaging of the viral vector. Examples include functions derived from adenovirus, baculovirus and/or herpes virus sufficient to result in packaging of the viral vector. For example, adenovirus helper functions will typically include adenovirus components E1a, E1b, E2a, E4, and VA RNA. The packaging functions may be supplied by infection of the packaging cell with the required virus. The packaging functions may be supplied together or separately to the packaging cell using a genetic construct such as a plasmid or an amplicon. See, e.g., pXR helper plasmids as described in Rabinowitz et al., 2002, J. Virol. 76:791, and pDG plasmids described in Grimm et al., 1998, Human Gene Therapy 9:2745-2760. The packaging functions may exist extrachromosomally within the packaging cell, but are preferably integrated into the cell's chromosomal DNA (e.g., E1 or E3 in HEK 293 cells).

Any suitable helper virus functions may be employed. For example, where the packaging cells are insect cells, baculovirus may serve as a helper virus. Herpes virus may also be used as a helper virus in AAV packaging methods. Hybrid herpes viruses encoding the AAV Rep protein(s) may advantageously facilitate for more scalable AAV vector production schemes.

Any method of introducing the nucleotide sequence carrying the helper functions into a cellular host for replication and packaging may be employed, including but not limited to, electroporation, calcium phosphate precipitation, microinjection, cationic or anionic liposomes, and liposomes in combination with a nuclear localization signal. In embodiments wherein the helper functions are provided by transfection using a virus vector or infection using a helper virus; standard methods for producing viral infection may be used.

Any suitable permissive or packaging cell known in the art may be employed in the production of the packaged viral vector. Mammalian cells or insect cells are preferred. Examples of cells useful for the production of packaging cells in the practice of the invention include, for example, human cell lines, such as VERO, WI38, MRC5, A549, HEK 293 cells (which express functional adenoviral E1 under the control of a constitutive promoter), B-50 or any other HeLa cells, HepG2, Saos-2, HuH7, and HT1080 cell lines. In one aspect, the packaging cell is capable of growing in suspension culture, more preferably, the cell is capable of growing in serum-free culture. In one embodiment, the packaging cell is a HEK293 that grows in suspension in serum free medium. In another embodiment, the packaging cell is the HEK293 cell described in U.S. Pat. No. 9,441,206 and deposited as ATCC No. PTA 13274. Numerous rAAV packaging cell lines are known in the art, including, but not limited to, those disclosed in WO 2002/46359. In another aspect, the packaging cell is cultured in the form of a cell stack (e.g. 10-layer cell stack seeded with HEK293 cells).

Cell lines for use as packaging cells include insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et al., Baculovirus Expression Vectors: A Laboratory Manual, Oxford Univ. Press (1994); Samulski et al., 1989, J. Virol. 63:3822-3828; Kajigaya et al., 1991, Proc. Nat'l. Acad. Sci. USA 88:4646-4650; Ruffing et al., 1992, J. Virol. 66:6922-6930; Kimbauer et al., 1996, Virol. 219:37-44; Zhao et al., 2000, Virol. 272:382-393; and Samulski et al., U.S. Pat. No. 6,204,059.

Virus capsids according to the invention can be produced using any method known in the art, e.g., by expression from a baculovirus (Brown et al., (1994) Virology 198:477-488). As a further alternative, the virus vectors of the invention can be produced in insect cells using baculovirus vectors to deliver the rep/cap genes and rAAV template as described, for example, by Urabe et al., 2002, Human Gene Therapy 13:1935-1943.

In another aspect, the present invention provides for a method of rAAV production in insect cells wherein a baculovirus packaging system or vectors may be constructed to carry the AAV Rep and Cap coding region by engineering these genes into the polyhedrin coding region of a baculovirus vector and producing viral recombinants by transfection into a host cell. Notably when using Baculovirus production for AAV, preferably the AAV DNA vector product is a self-complementary AAV like molecule without using mutation to the AAV ITR. This appears to be a by-product of inefficient AAV rep nicking in insect cells which results in a self-complementary DNA molecule by virtue of lack of functional Rep enzyme activity. The host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids.

During production, the packaging cells generally include one or more viral vector functions along with helper functions and packaging functions sufficient to result in replication and packaging of the viral vector. These various functions may be supplied together or separately to the packaging cell using a genetic construct such as a plasmid or an amplicon, and they may exist extrachromosomally within the cell line or integrated into the cell's chromosomes.

The cells may be supplied with any one or more of the stated functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA.

The rAAV vector may be purified by methods standard in the art such as by column chromatography or cesium chloride gradients. Methods for purifying rAAV vectors are known in the art and include methods described in Clark et al., 1999, Human Gene Therapy 10(6): 1031-1039; Schenpp and Clark, 2002, Methods Mol. Med. 69:427-443; U.S. Pat. No. 6,566,118 and WO 98/09657.

### Methods of Delivering Nucleic Acids Encoding Anti-C3 Antibodies to the Retina

In several embodiments, methods for delivering heterologous nucleotide sequences encoding anti-C3 and/or anti-C5 antibodies to the retina are provided utilizing an rAAV as herein described. The rAAV may be employed to deliver a nucleotide sequence encoding the antibody to a retinal cell in vitro, e.g., to produce an antibody polypeptide or nucleic acid in vitro for ex vivo gene therapy. The rAAV are additionally useful in a method of delivering a nucleotide sequence to a subject in need thereof, e.g., to express the antibody in a subject in need thereof, such as human with dry AMD or a human with geographic atrophy. In this manner, the antibody may thus be produced in vivo in the subject to restore complement regulation.

Thus, in one aspect, a method of delivering a nucleic acid encoding an anti-C3 and/or anti-C5 antibody or antigen-binding fragment thereof to a retinal cell is provided, the method comprising contacting the retinal cell with an rAAV virion as herein described.

In another aspect, a method of delivering a nucleic acid encoding an anti-C3 and/or anti-C5 antibody or antigen-binding variant thereof to a retinal cell in a mammalian subject is provided, the method comprising administering an effective amount of the rAAV virion as herein described or a pharmaceutical formulation comprising same to a mammalian subject.

The rAAV may be administered to retina of a subject by any suitable route. In preferred embodiments, the rAAV is administered to the subject intraocularly, preferably by subretinal, suprachoroidal, and/or intravitreal injection. In some particularly preferred embodiments, rAAV is administered to a subject via intravitreal injection, more preferably by a single intravitreal injection.

### Treatment Methods

In certain embodiments, a method is provided for the treatment of dry AMD in a subject in need of such treatment, the method comprising administering to the subject a recombinant adeno-associated virus (rAAV) comprising: (a) a variant AAV capsid protein comprising a heterologous peptide insertion with a length of 7 to 20 amino acids covalently inserted in the GH-loop of the AAV capsid protein, wherein the peptide insertion comprises the amino acid sequence ISDQTKH (SEQ ID NO:105) and (ii) a heterologous nucleic acid comprising a nucleotide sequence encoding an anti-C3 and/or anti-C5 antibody or antigen-binding variant thereof as herein described, said nucleotide sequence operably linked to a promoter, or administering to the subject a pharmaceutical composition comprising said rAAV and a pharmaceutically acceptable carrier, preferably wherein said rAAV or said pharmaceutical composition is administered to the subject by intravitreal injection. Also provided is the use of said rAAV or a pharmaceutical composition comprising same for the treatment of dry AMD. Also provided is the use of said rAAV in the manufacture of a medicament for the treatment of dry AMD.

In related embodiments, a method is provided for the treatment of a geographic atrophy in a subject in need of such treatment, the method comprising administering to the subject a recombinant adeno-associated virus (rAAV) virion comprising: (a) a variant AAV capsid protein comprising a heterologous peptide insertion with a length of 7 to 20 amino acids covalently inserted in the GH-loop of the AAV capsid protein, wherein the peptide insertion comprises the amino acid sequence ISDQTKH (SEQ ID NO:168) and (ii) a heterologous nucleic acid comprising a nucleotide sequence encoding an anti-C3 and/or anti-C5 antibody or antigen-binding variant thereof as herein described, said nucleotide sequence operably linked to a promoter, or administering to the subject a pharmaceutical composition comprising said rAAV virion and a pharmaceutically acceptable carrier, preferably wherein said rAAV or said pharmaceutical composition is administered to the subject by intravitreal injection. Also provided is the use of said rAAV or a pharmaceutical composition comprising same for the treatment of geographic atrophy. Also provided is the use of said rAAV in the manufacture of a medicament for the treatment of geographic atrophy.

In some aspects, the variant AAV capsid protein comprises an amino acid sequence at least 80%, at least 85%, at least 90%, at least 95%, at least 98%, at least 99% identical to, or is 100% identical to, the amino acid sequence set forth as SEQ ID NO:170 and comprises a P34A substitution and an LAISDQTKHA (SEQ ID NO:169) peptide insertion at amino acid position 588.

In some preferred aspects, the heterologous nucleic acid encapsulated by the variant AAV capsid protein comprises a nucleotide sequence that encodes an antibody comprising one or more CDRs comprising the amino acid sequence according to Table 1 or Table 2 or comprising an amino acid sequence at least 75% identical thereto. In a preferred embodiment, the antibody comprises at least one, more preferably at least two, most preferably three CDRs, each comprising an amino acid sequence selected from those set forth as SEQ ID Nos: 54-56 or comprising an amino acid sequence at least 75% identical to an amino acid sequence selected from those set forth as SEQ ID Nos: 54-56. In related aspects, the nucleotide sequence encodes an antibody comprising an amino acid sequence selected from those set forth as SEQ ID Nos: 14 and 133-136 or comprising an amino acid sequence at least 75% identical (e.g., at least 80%, at 90%, at least 95%, at least 98%, or at least 99%) identical to an amino acid sequence selected from those set forth as SEQ ID Nos: 14 and 133-136. In other related aspects, the nucleotide sequence encoding the nanobody comprises the nucleotide sequence set forth as any one of SEQ ID Nos: 133-136. In related aspects, the nucleotide sequence encodes an antibody comprising an amino acid sequence selected from those set forth as SEQ ID Nos: 137-163 or comprising an amino acid sequence at least 75% identical (e.g., at least 80%, at 90%, at least 95%, at least 98%, or at least 99% identical) to an amino acid sequence selected from those set forth as SEQ ID Nos: 137-163.

In some aspects, the rAAV is administered by periocular, intravitreal, suprachoroidal and/or subretinal injection, preferably by intravitreal injection, to a subject with dry AMD and/or geographic atrophy at a dose of from about 1×10⁸ vector genomes (vg)/eye to about 1×10¹³ vg/eye, from about 1×10⁹ vg/eye to about 1×10¹² vg/eye, from about 1×10⁹ vg/eye to about 1×10¹¹ vg/eye or from about 6×10⁹ vg/eye to about 6×10¹⁰ vg/eye.

In some embodiments, a method for treating dry AMD and/or geographic atrophy in a subject in need thereof is provided comprising administering to the subject by periocular, intravitreal, suprachoroidal and/or subretinal injection, an effective amount of an rAAV virion comprising (i) a capsid comprising a capsid protein comprising the amino acid sequence set forth as SEQ ID NO: 170 and (ii) a heterologous nucleic acid comprising from 5′ to 3′: (a) an AAV2 terminal repeat (b) a CAG or CMV promoter (c) a nucleotide sequence encoding an amino acid sequence as set forth in any of SEQ ID Nos: 14 and 133-136 (d) a polyadenylation sequence and (e) an AAV2 terminal repeat.

In related embodiments, a method for treating dry AMD and/or geographic atrophy in a subject in need thereof is provided comprising administering to the subject by periocular, intravitreal, suprachoroidal and/or subretinal injection, a pharmaceutical composition comprising a pharmaceutically acceptable carrier and an rAAV virion comprising (i) a capsid protein comprising the amino acid sequence set forth as SEQ ID NO:170 and (ii) a heterologous nucleic acid comprising from 5′ to 3′: (a) an AAV2 terminal repeat (b) a CAG or CMV promoter (c) a nucleotide sequence encoding an amino acid sequence as set forth in any of SEQ ID Nos: 14 and 133-136 (d) a polyadenylation sequence and (e) an AAV2 terminal repeat. In some aspects, the pharmaceutical composition comprises between about 1×10⁸ vg to about 1×10¹³ vg, between about 1×109 vg to about 1×10¹² vg, between about 1×10⁹ vg to about 1×10¹¹ vg or between about 6×10⁹ vg to about 6×10¹⁰ vg.

In some embodiments, a method for treating dry AMD and/or geographic atrophy in a subject in need thereof is provided comprising administering to the subject by periocular, intravitreal, suprachoroidal and/or subretinal injection, an effective amount of an rAAV virion comprising (i) a capsid comprising a capsid protein comprising the amino acid sequence set forth as SEQ ID NO: 170 and (ii) a heterologous nucleic acid comprising from 5′ to 3′: (a) an AAV2 terminal repeat (b) a CAG or CMV promoter (c) a nucleotide sequence encoding an amino acid sequence as set forth in any of SEQ ID Nos: 137-163 (d) a polyadenylation sequence and (e) an AAV2 terminal repeat.

In related embodiments, a method for treating dry AMD and/or geographic atrophy in a subject in need thereof is provided comprising administering to the subject by periocular, intravitreal, suprachoroidal and/or subretinal injection, a pharmaceutical composition comprising a pharmaceutically acceptable carrier and an rAAV virion comprising (i) a capsid protein comprising the amino acid sequence set forth as SEQ ID NO:170 and (ii) a heterologous nucleic acid comprising from 5′ to 3′: (a) an AAV2 terminal repeat (b) a CAG or CMV promoter (c) a nucleotide sequence encoding an amino acid sequence as set forth in any of SEQ ID Nos: 137-163 (d) a polyadenylation sequence and (e) an AAV2 terminal repeat. In some aspects, the pharmaceutical composition comprises between about 1×10⁸ vg to about 1×10¹³ vg, between about 1×10⁹ vg to about 1×10¹² vg, between about 1×10⁹ vg to about 1×10¹¹ vg or between about 6×10⁹ vg to about 6×10¹⁰ vg.

Pharmaceutical compositions comprising an rAAV as described herein are provided. In some embodiments, the pharmaceutical composition comprises about 1×10⁸ to about 1×10¹⁴ vector particles or vector genomes, about 1×10⁸ to about 1×10¹³ vector particles or vector genomes, about 1×10⁹ to about 1×10¹² vector particles or vector genomes, or about 1×10⁹, about 2×10⁹, 3×10⁹, about 4×10⁹, about 5×10⁹, about 6×10⁹, about 7×10⁹, about 8×10⁹, about 9×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹ or about 1×10¹² vector particles or vector genomes. In some aspects, the pharmaceutical composition comprises about 1×10⁹ to about 1×10¹¹ vg, and preferably comprises about 6×10⁹ vg to about 6×10¹⁰ vg. In some preferred embodiments, the pharmaceutical composition is administered to a human with dry AMD and/or geographic atrophy via intravitreal injection.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

Data from human dry and wet age-related macular degeneration (AMD) patients supports the hypothesis that constant ‘tickover’ of the alternative complement pathway results in chronic deposition of the complement membrane attack complex (MAC) on the choriocapillaris and the retinal pigment epithelium (RPE). Sub-lytic levels of MAC lead to the production of cytokines and inflammatory molecules. Prevention of MAC deposition thus provides an effective treatment for dry age-related macular degeneration and geographic atrophy.

Nanobodies against human complement factor C3 (SEQ ID Nos: 1-14) were isolated through phage display panning against human C3 protein and tested for their ability to specifically bind to C3 and inhibit complement. Recombinant AAV comprising a capsid of SEQ ID NO: 107 was generated encapsulating a heterologous nucleic acid encoding a representative anti-C3 nanobody and assessed for complement inhibition.

### Materials and Methods

### Recombinant Protein Production

Recombinant protein was produced in HEK293 cells by NeoClone and GeneScript for each nanobody identified.

### Cell Culture

HEK293T cells were grown on tissue culture plastic at 5% CO₂ in normoxic conditions in DMEM, 10% fetal bovine serum (FBS), and with 1% Penicillin/Streptomycin.

iPSC-RPE were grown on Matrigel coated plates and matured for 30 days at 5% CO₂ in normoxic conditions in XVIVO-10 media.

### Transduction

iPSC-RPE were transduced ≥30 days after seeding with a multiplicity of infection (MOI, vg/cell) of 5,000, 10,000 or 20,000. Samples were collected 7 days post infection for analysis.

### Complement Inhibition Assay

The Wieslab® Complement system Alternative pathway kit (Eagle Biosciences Inc. (Cat. No. COMPL AP 330) is an enzyme immunoassay for the qualitative determination of functional alternative complement pathway in human serum and was executed according to manufacturer's instructions. The Wieslab Complement assay combines the specific activation of the pathway with the use of labeled antibodies specific for a neoepitope of the termina complement complex, C5b-9, produced as the result of complement activation (the amount of C5b-9 generated is proportional to the functional activity of the complement system). Briefly, for the Wieslab complement inhibition assay performed in Table 2, normal human or cynomolgus serum (Complement Tech) was diluted 1:20, along with 3.75 μg of purified nanobodies. Control and test serum was added at 100 μL/well to the provided plates. After incubating at 37 C for 1 hour, plates were washed 3 times with washing solution. Prepared conjugate alkaline phosphatase-labelled antibodies to C5b-9 was added and incubated for 30 minutes at room temperature. After washing, substrate solution was added to each well and absorbance read at 405 nm.

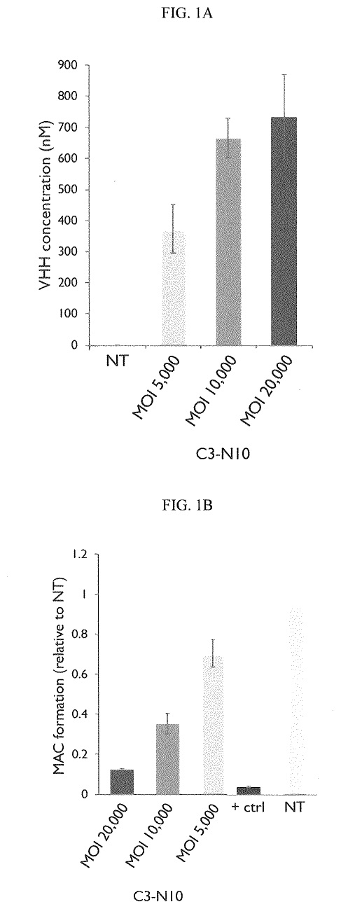

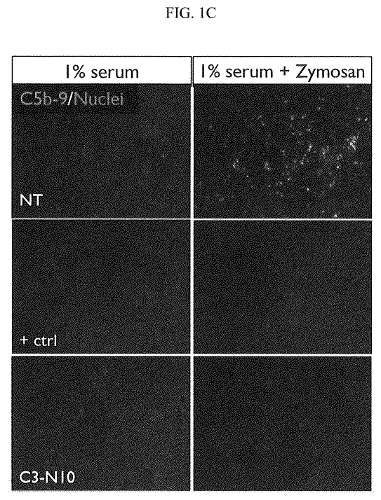

For the Wieslab complement inhibition assay performed in FIG. 1, supernatant following transfection, or recombinant purified nanobodies for the dose response curve, normalized to 1 mg/ml, were diluted in the dilution buffer provided in the assay kit. A 5% non-human primate (NHP) serum was prepared in the dilution buffer as per the protocol and 100 μl of the 5% NHP serum was transferred to a microtiter plate. The nanobody dilutions were applied in triplicates to the plate and mixed for 5 minutes on the plate mixer. Following mixing, the serum with added nanobody dilutions were transferred to assay plate and incubated for 60 min at 37° C. Post incubation, the plate was washed 3 times with the wash buffer provided in the kit and incubated with the conjugate for 30 min at room temperature. Following incubation, the plate was washed 3 times with the wash buffer provided. One hundred micro-liter of the substrate solution provided with the kit was added to the microtiter plate and absorbance was read at 405 nm every 5 min intervals for 60 minutes. For data analysis the OD 405 nm at 60 min was plotted as a function of concentration for the dose response curve.

### Immunocytochemistry (ICC)

iPSC-RPE were exposed to either 1% human serum or 1% human serum plus 5 μg/ml Zymosan to induce the alternative complement pathway. Twenty-four hours post stimulation, cells were fixed with 4% paraformaldehyde and then permeabilized with 0.01% Triton X-100. Primary antibody (C5b-9) was added for 18 hours at 4° C., followed by a secondary antibody and DRAQ5 nuclear counterstain for 1 hour at room temperature.

rAAV Manufacturing

For in vitro studies, rAAV with the capsid amino acid sequence listed in SEQ ID NO: 107 was produced via PEI-mediated triple transfection into HEK293 cells. Cells were cultured in DMEM supplemented with FBS and maintained at 37° C. in a 5% CO₂ environment.

Briefly, cells were seeded into CellSTACK vessels and transfected with pHelper, the relevant Rep-Cap plasmids, and the CAG-nanobody payload plasmid. Three to four days following transfection, cells and supernatant were collected and lysed, endonuclease treated, clarified via sterile filtration, and frozen. Thawed harvest material was clarified and loaded onto relevant affinity resins, eluted at low pH and immediately neutralized. Purified lots were formulated in either a phosphate or Tris-based buffer system containing NaCl and 0.005% Pluronic F-68. Purified lots were stored at −80° C. until use.

### ELISA Binding Assay

Antigen protein was pre-coated in standard ELISA plates at 500 ng/well then blocked with 1% BSA. Nanobodies were diluted to 10 ug/mL and 150 uL were added to each well. After incubation and washing, remaining bound nanobodies were detected with HRP conjugated monoclonal rabbit anti-camelid VHH. Control wells with no nanobodies was used as background and this reading was subtracted from test wells.

### Octet/Gator Binding Affinity Measurement

Biolayer Interferometry via the Gator or Octet machines were used to determine the binding affinities of nanobodies to their respective antigens. The anti-His probes were first equilibrated in buffer (PBST+5% BSA) at 37 C and at 1000 rpm in a 96 well plate. After equilibration, the 6×His tagged nanobodies (load sample) were immobilized onto the sensors. Once the probes reached 60% capacity, the loaded sensors were washed in buffer to remove any non-specifically binding. The nanobody loaded probes are then immersed in a serial dilution of untagged target antigen protein (analyte sample) for the association step to obtain “Kon”. The sensor is then transferred to a blank buffer to obtain off rate “Koff”, dissociation rate constant (1/s). The ratio of Koff/Kon is the “KD” is the equilibrium dissociation rate constant which is the direct measure of binding affinity of the binding pair.

### Results

### Nanobody Target Binding and Complement System Inhibition

TABLE 3

| | | Hum C3 | | | | | |
|---|---|---|---|---|---|---|---|
| | | times | Cy-C3 times | | | | |
| | | Background | Background | | | KD | KD |
| | | ELISA | ELISA | Wieslab | Wieslab | to | to |
| | Mammalian | Binding | Binding | Inibition | Inhibition | huC3 | cyC3 |
| Clone ID | Production | Assay | Assay | 4 uM | 0.1 uM | (nM) | (nM) |
| C3I3 | 215 ug | 12.9 | 1.2 | 0.090 | 2.393 | 17 | 39 |
| C3I4 | 176 ug | 29.2 | 20.6 | 0.092 | 3.192 | 27 | 13 |
| C3I8 | 138 ug | 3.9 | 6.2 | 0.093 | 2.789 | 4.8 | 5 |
| C3I22 | 225 ug | 2.2 | 5 | 0.103 | 2.883 | 3.9 | 5.6 |
| C3N10 | 122 ug | 6.2 | 6.1 | 0.092 | 0.116 | 7.6 | |
| C3S3 | 827 ug | 43.2 | 14.3 | 0.095 | 0.114 | 23 | 12 |
| C3S4 | 566 ug | 3.6 | 1 | 2.897 | | | |
| C3S5 | 366 ug | 2.2 | 1 | | | | |
| C3S6 | 839 ug | 25.2 | 2.5 | 0.12 | 2.569 | 20 | 47 |
| C3S7 | 281 ug | 8.4 | 1.1 | 0.242 | 2.982 | NB | NB |
| C3S8 | 816 ug | 46.3 | 21 | 0.129 | 3.096 | 14 | 19 |
| C389 | 165 ug | 17.3 | 1 | 0.1 | 2.909 | WkB | NB |
| C3S10 | 753 ug | 1 | 1 | | | | |

NB = Not Bound;
WkB = Weak Binding

In Table 3, the nanobody clones isolated through phage display panning against human C3 protein are listed along with their respective characteristics (the corresponding amino acid sequences are as described). Production yields are calculated by the amount of purified protein extracted from 60 mL supernatants of transiently transfected HEK293 suspension cell lines. ELISA binding to human or cynomolgus C3 protein is the fold above background each clone's signal over no antibody. Wieslab Complement Alternative Pathway Assay results are determined by whether each nanobody was capable of inhibiting at least 50% complement activation at the designated concentrations. Finally, KD values for VHH binding to human or cynomolgus C3 protein using BLI.

All nanobodies that were assayed in Table 3, other than C3S5 and C3S10, bound either human and/or cynomolgus C3 protein (see corresponding ELISA binding and KD values). Similarly, all nanobodies that were assayed in Table 3, other than C3S4, inhibited MAC formation either at 4 μM and/or 0.1 μM as shown in their corresponding Wieslab Inhibition columns.

Transduction of rAAV Containing Nanobody Transgene Inhibits MAC Formation in iPSC-RPE

Induced pluripotent stem cell derived retinal pigmented epithelial cells (iPSC-RPE) were transduced with rAAV with the capsid amino acid sequence listed in SEQ ID NO:107 carrying a C3-N10 transgene (SEQ ID NO:14) at three multiplicities of infection, (MOI) 5,000, 10,000 and 20,000. Seven days post transduction, nanobody (VHH) protein levels were analyzed in supernatants via ELISA and a dose response was observed in expression (FIG. 1a). Complement inhibition was assessed using supernatants to determine MAC formation. A dose response in MAC formation was observed compared to non-transduced following transduction at MOI 5,000, 10,000 and 20,000, with a higher MOI yielding less MAC formation (FIG. 1b). In addition, MAC deposition onto cells following an alternative pathway stimulus, Zymosan, was observed, which was reversed when cells had been transduced with C3-N10 rAAV or a positive control for alternate complement pathway inhibition (FIG. 1c).

### Example 2

Data from human dry and wet age-related macular degeneration (AMD) patients supports the hypothesis that constant ‘tickover’ of the alternative complement pathway results in chronic deposition of the complement membrane attack complex (MAC) on the choriocapillaris and the retinal pigment epithelium (RPE). Sub-lytic levels of MAC lead to the production of cytokines and inflammatory molecules. Prevention of MAC deposition thus provides an effective treatment for dry age-related macular degeneration and geographic atrophy.

Nanobodies against human complement factor C5 (SEQ ID Nos: 57-65) were isolated through phage display panning against human C protein and tested for their ability to specifically bind to C5 and inhibit complement.

### Materials and Methods

### Complement Inhibition Assay

The Wieslab® Complement system Alternative pathway kit (Eagle Biosciences Inc. (Cat. No. COMPL AP 330) is an enzyme immunoassay for the qualitative determination of functional alternative complement pathway in human serum and was executed according to manufacturer's instructions. The Wieslab Complement assay combines the specific activation of the pathway with the use of labeled antibodies specific for a neoepitope of the termina complement complex, C5b-9, produced as the result of complement activation (the amount of C5b-9 generated is proportional to the functional activity of the complement system). Briefly, for the Wieslab complement inhibition assay performed in Table 2, normal human or cynomolgus serum (Complement Tech) was diluted 1:20, along with 3.75 ug of purified nanobodies. Control and test serum was added at 100 uL/well to the provided plates. After incubating at 37 C for 1 hour, plates were washed 3 times with washing solution. Prepared conjugate alkaline phosphatase-labelled antibodies to C5b-9 was added and incubated for 30 minutes at room temperature. After washing, substrate solution was added to each well and absorbance read at 405 nm.

### ELISA Binding Assay

Antigen protein was pre-coated in standard ELISA plates at 500 ng/well then blocked with 1% BSA. Nanobodies were diluted to 10 ug/mL and 150 uL were added to each well. After incubation and washing, remaining bound nanobodies were detected with HRP conjugated monoclonal rabbit anti-camelid VHH. Control wells with no nanobodies was used as background and this reading was subtracted from test wells.

### Octet/Gator Binding Affinity Measurement

Biolayer Interferometry via the Gator or Octet machines were used to determine the binding affinities of nanobodies to their respective antigens. The anti-His probes were first equilibrated in buffer (PBST+5% BSA) at 37 C and at 1000 rpm in a 96 well plate. After equilibration, the 6×His tagged nanobodies (load sample) were immobilized onto the sensors. Once the probes reached 60% capacity, the loaded sensors were washed in buffer to remove any non-specifically binding. The nanobody loaded probes are then immersed in a serial dilution of untagged target antigen protein (analyte sample) for the association step to obtain “Kon”. The sensor is then transferred to a blank buffer to obtain off rate “Koff”, dissociation rate constant (1/s). The ratio of Koff/Kon is the “KD” is the equilibrium dissociation rate constant which is the direct measure of binding affinity of the binding pair.

### Results

### Nanobody Target Binding and Complement System Inhibition

TABLE 4

| | | Hum C5 | | | | |
|---|---|---|---|---|---|---|
| | | times | | | | |
| | | Background | Cy-C5 times | | | |
| | | ELISA | Background | Wieslab | Wieslab | KD to |
| | Mammalian | Binding | ELISA Binding | Inibition | Inhibition | huC5 |
| Clone ID | Production | Assay | Assay | 4 uM | 0.1 uM | (nM) |
| C5S1 | 239 ug | 35.5 | 1.1 | 1.396 | 3.048 | 9.5 |
| C5S2 | 486 ug | 46 | 1.1 | 0.1295 | 0.303 | 1.98 |
| CSS3 | 225 ug | 8.8 | 1 | 2.5515 | | |
| C5S4 | 23 ug | 8.3 | 1.1 | 2.564 | | |
| C5S5 | 231 ug | 24.3 | | 1.2295 | 3.061 | 13.0 |
| C5S6 | 129 ug | 3.3 | 1 | | | |
| C5S7 | 119 ug | 8.6 | 1 | 2.3255 | | |
| C5S8 | 320 ug | 49.6 | 1 | 0.253 | 1.554 | 7.0 |
| C5S9 | 482 ug | 50.8 | 1 | 0.092 | | |
| C5S12 | 96 ug | 2.7 | 2.2 | | | |

NB = Not Bound;
WkB = Weak Binding

In Table 4, the nanobody clones isolated through phage display panning against human C5 protein are listed along with their respective characteristics. Production yields are calculated by the amount of purified protein extracted from 60 mL supernatants of transiently transfected HEK293 suspension cell lines. ELISA binding to human C5 or cynomolgus C5a protein is the fold above background each clone's signal over no antibody. Wieslab Complement Alternative Pathway Assay results are determined by whether each nanobody was capable of inhibiting at least 25% complement activation at the designated concentrations. Finally, KD values for VHH binding to human C5 protein using BLI.

All nanobodies that were assayed in Table 4, other than C5S12, bound human C5 protein (see corresponding ELISA binding and KD values). Similarly, all nanobodies that were assayed in Table 4, other than C5S3, C5S4 and C5S7 inhibited MAC formation either at 4 uM and/or 0.1 uM as shown in their corresponding Wieslab Inhibition columns.

### Example 3

### Methods and Materials

### Recombinant Complement Protein Production

For recombinant protein production, both the wild type and mutant complement sequences were cloned with a C-terminal TEV cleavage site and 6×His tag into pcDNA plasmid with a mouse IgG secretory sequence and transiently transfected into Expi293 cells. The cells were harvested, spin at 250×g for 30 min and the supernatant collected. The recombinant protein was purified by single step purification by using Immobilized metal affinity chromatography (IMAC) affinity chromatography. For purification, the clarified supernatant was passed through a 0.2-micron filter and passed through a 1 ml His excel trap column pre-equilibrated with equilibration buffer (50 mM Tris, 150 mM NaCl pH 8.0) at 1 mL/min flow rate. Following capture step, the column was washed with a wash buffer (10 mM Imidazole, 50 mM Tris, 150 nM NaCl pH 8) and eluted with his tag elution buffer (250 nM Imidazole, 50 mM Tris, 150 mM NaCl pH 8). The his-elution was subjected to TEV protease for 6×His tag cleavage. Post cleavage, the 6×His tag is removed by passing the eluate through IMAC resin. The flowthrough from IMAC column is passed size exclusion chromatography to buffer exchange into PBS (137 mM NaCl, 2.7 mM KCl, 4.3 mM NaH2PO4, 1.4 Mm KH2PO4) and passed through 0.4 nm filter and stored at −20C in aliquots.

### Recombinant VHH Production

For recombinant nanobody production both the wild type and humanized nanobody sequences were cloned with a C-terminal strep II tag and 6×His tag into pcDNA plasmid with a mouse IgG secretory sequence and transiently transfected into Expi293 cells. The cells were harvested, spin at 250×g for 30 min and the supernatant collected. The recombinant nanobody was purified by single step purification by using IMAC affinity chromatography. For purification the clarified supernatant was passed through a 0.2 micron filter and passed onto a 1 ml His excel trap column pre-equilibrated with equilibration buffer (50 mM Tris, 150 mM NaCl pH 8.0) at 1 ml/min flow rate. Following capture step, the column was washed with a wash buffer (10 mM Imidazole, 50 mM Tris, 150 nM NaCl pH 8) and eluted with his tag elution buffer (250 nM Imidazole, 50 mM Tris, 150 mM NaCl pH 8). The his-elution subjected to buffer exchange into PBS and passed through 0.4 nm filter and stored at −20C in aliquots.

### Recombinant VHH-Fc Production

For recombinant production of the Fc fused nanobodies, the VHH cDNAs were cloned into pCDNA3 with a C-terminal human IgG2 hinge-human IgG4 Fc to express the nanobodies as Fc dimers in Expi293 cells with a mouse IgG signal peptide for secretion in the supernatants. Post transient transfection, the cells were harvested and centrifuged at 250×g for 30 min to pellet the cells and supernatant and pass through a 0.45 micron membrane. The recombinant VHH-Fc dimer was passed through Mabselect sure column at pH 7 in 1×PBS buffer to capture the VHH-Fc fusion. Post loading the column is washed with wash buffer and eluted using a low pH buffer, 0.1M Citrate buffer, 150 mM NaCl pH 3. The eluate was neutralized immediately with 1 M Tris pH 8 followed by buffer exchanging into 1×PBS pH 7 and aliquoted.

### Wieslab Complement Activation Assays

The Wieslab alternative complement pathway assay system was employed for semiquantitative estimation of the inhibitory activity of the nanobodies. The Wieslab Alternative pathway kit is an enzyme immunoassay for the qualitative determination of functional alternative complement pathway in human serum. The assay combines the principles of hemolytic assay for complement activation with the use of labeled antibodies specific for neoantigen produced because of complement activation. In the complement activation AP, the assay plate is coated with specific activators of alternate pathway activators and the diluent solution provided contains specific blocker to ensure activation of only alternate pathway. To evaluate nanobody complementary AP inhibitory effect, normal human serum was diluted 1:20 in the diluent provided and added to a 96 well plate, recombinantly purified nanobody was spiked in the wells in duplicates along with control and test serum and mixed on a plate shaker for 5 min at 400 rpm. Following mixing the contents of the 96 well plate is transferred into the assay plate using a multichannel pipette. After incubating at 37 C for 1 hour, plates were washed 3 times with washing solution and conjugate alkaline phosphatase-labelled antibodies to C5b-9 was added and incubated for 30 minutes at room temperature. After washing, substrate solution was added to the wells and incubated at room temperature for 45 minutes. The amount of complement activation correlates with the color intensity and is measured in terms of absorbance/optical density at 405 nm.

### Classical and Lectin Pathway Inhibition Assay

Additionally, complement inhibitory action of nanobodies was investigated in the classical and lectin pathways using the Wieslab classical & mannan-binding lectin pathway kits. In the classical pathway kit, the microplate wells are coated with CP activators like the C1q/C1 complex, and the diluent supplied contains specific activators of classical pathway. Similarly, in case of lectin pathway MBL kit, consists of micro well plate coated with mannose binding lectins, opsonin or ficolin that specifically activate lectin pathway in combination with diluent buffer with specific blockers for the AP & CP.

### Primate Serum Cross Reactivity

Cross reactivity to primate serum was also investigated by employing Wieslab AP assay kit. In this case, the nanobody was spiked in a 1:20 dilution of normal cynomolgus monkey serum and followed by executing the standard manufacturer guidelines.

### IC50 Calculation

The half-maximal inhibitory concentration membrane associated complex formation (IC50 MAC) was determined by using Wieslab alternate pathway assay. A dilution series of the recombinant purified nanobody was spiked in a 20× diluted human serum at various concentrations and applied to the Wieslab assay plate. Following a 60-minute incubation washed and treated with the conjugate alkaline phosphate labelled with C5b-9 and incubated for 30 minutes. Following conjugate incubation, the plate was washed and incubated with substrate solution and read at 405 nm at 45 min time point. The IC50 MAC formation was estimated by curve fitting software.

### Mouse Alternative Complement Pathway Assay

Mouse C3/C5 Elisa was employed to check the cross functional activity of the C3 and C5 nanobodies to mouse complement proteins. The assay provides a micro well plate coated with LPS for specific activation of the alternative pathway. For this assay, the mouse serum was diluted 1:20 in the dilution buffer provided and transferred to 96 well plate and the serial dilutions of the nanobodies were spiked in the serum in triplicates along with a positive control mouse BB5.1 antibody. The 96 well plate was agitated for 5 min on plate shaker at 400 rpm and the contents of the plate transferred to the micro well plate coated with LPS and incubated at 37 C for 60 min. Post incubation the plate was washed 4 times with the wash buffer provided and biotinylated tracer antibody was added and incubated for another 60 min at 37 C followed by another 4 washes. Streptavidin peroxide conjugate was added and incubated for 60 min at 37 C followed by 4 washes. A TMB solution was added and incubated for 30 min at room temperature and a stop solution was added to stop the reaction and OD measured at 450 nm.

### Gator Assay for Binding Affinity (KD)

The anti-strep II antibody coated probes were used for binding affinity determination by Octet/Gator. The anti-strep II antibody probe was immersed in buffer (PBST+5% BSA) for equilibration at 37 C and at 1000 rpm in a 96 well plate. Following equilibration, the probes were immersed in well containing VHH-strep-tagged VHH for loading. Once the probes are about 60% loaded, the anti-strep II antibody probes were immersed in a buffer well for a washing step to remove any non-specifically bound VHH to the probes. Followed by washing the probes are then immersed in a serial dilution of either recombinantly purified human or cynomolgus C3 or C5 wild type or the C5 single and double mutant proteins (untagged) for association step to obtain “Kon” which is the association rate constant (1/s). After association a dissociation step is performed in buffer to obtain off rate “Koff”, dissociation rate constant (1/s). The ratio of Koff/Kon is the “KD” which is the equilibrium dissociation rate constant which is the direct measure of binding affinity of the binding pair.

### Antibody Cross-Blocking to C5 Protein by Gator BLI Sandwich Assay

Gator BLI platform was used to investigate binding epitope(s) of C5S2.2 nanobody. Like eculizumab, C5S2.2 nanobody did not show cross binding to cynomolgus C5 protein nor have inhibitory alternative complement activity in cynomolgus serum. Hence binding epitope was interrogated to find out whether eculizumab and C5S2.2 cross-block each other to human C5. A sandwich approach was used on BLI where anti-human Fc-binding probes were used to immobilize eculizumab for 1-2 min. After washing the pre-loaded probes were then dipped into untagged C5 alone or C5 preincubated with C5S2 in the recommended binding buffer for 5-6 min to allow for association of the either the C5 or C5S2-C5 complex.

### Reverse Sandwich Assay

A reverse sandwich was performed by immobilizing dilution series of C5S2 nanobody on the anti-strep-II probes. After washing, this pre-loaded probe was immersed into C5 containing binding solution for an to check for letting the C5 protein associate with immobilized nanobody for 2-3 min. Once the C5 protein is associated the probe holding the VHH-C5 complex was immersed in the well containing eculizumab for 5-6 minutes for association.

### Epitope Mapping by Cross-Linking Mass Spectrometry (XL-MS)

Epitope peptide mapping via cross-linking mass spectrometry of the humanized C3 nanobodies against human C3 protein was performed. Purified nanobodies and recombinant proteins were generated. Each nanobody could bind to target then chemically cross-linked using disuccinimidyl suberate. This complex was then treated with a set of overlapping proteases and the peptides were analyzed via mass spectrometry and subsequently compared to native protein sequences.

### QC Check for Sample Integrity

As part of initial QC check, High Mass MALDI MS analysis was performed on the recombinant protein both nanobody, C3 & C5. Additionally, the same experiment was performed on cross linked recombinant proteins and compared to verify sample integrity and aggregation level which also serves as control for the subsequent experiments.

### Characterizing Nanobody/C3 Complexes

Once sample integrity is confirmed, the C3N10/C3 & C3S3/C3 were characterized. For characterizing, complexes were made by incubating at two different concentrations following which measurements were performed by Autoflex MALDI/TOF mass spectrometer. For cross linking experiment the C3N10/C3 & C383/C3 complexes were treated with DDS for 180 minutes and measured on Autoflex MALDI/TOF. The control and crosslinked mass spectrometry read out was compared for any additional peaks.

### Peptide Mass Finger Printing

For peptide mass finger printing hC3 was cross linked with DDS for 180 min and cleaved by five different proteases (trypsin, Chymotrypsin Elastase, Asp-N and Thermolysin) and measured on for maximum sequence coverage. Post cleavage both the control and crosslinked samples are subjected to Liquid chromatography for peptide separation followed by mass analysis on Q-Exactive Plus mass spectrometer. Resulting peptides were analyzed by using CovalX proprietary software and peptide fingerprint map for both the cross linked and control sample was generated.

Anti-Drug-Antibody Response to Nanobodies from Normal Human Donor Serum Using Meso Scale Discovery Immunoassays

Purified nanobodies and controls were passively coated onto MSD LISXA-3 plates at 1 ug/mL then blocked with casein. An in-house repository of serum and plasma from normal human donors was diluted 1:100 and applied to each well then incubated for 2 hours at room temperature with shaking to allow binding. After washing, the bound antibodies were detected using Sulfo-TAG goat-anti-human IgG. Purified human IgG was coated directly at multiple concentrations to track assay performance and as positive control. After washing, diluted reading buffer was added to each well and plates were read in a Meso Sector S 600.

### Plasmid DNA Cloning and Isolation

An AAV supportive vector containing the CAG promoter and other necessary AAV cassette components, was digested with restriction enzymes to remove the undesired payload. Custom gblocks (GB) from Integrated DNA Technologies containing the desired VHH were cloned into the backbone,

Miniprep cultures were grown from the resulting colonies following cloning and transformation, in Terrific Broth, supplemented with Kanamycin Sulphate at 100 mg/mL and grown at 30° C. overnight with shaking. DNA was isolated with QIAprep Spin Miniprep Kit following manufacturer's protocol and sequenced using Primordium Lab's sequencing technology to identify positive clones of sCFH and CFH.

### Cell Culture

HEK293T cells were grown in DMEM media with 1% Penicillin/Streptomycin and 10% fetal bovine serum. Cells were passaged twice a week and not used beyond passage 20.

iPSC-RPE were grown on Matrigel coated plates and matured for 30 days at 5% CO₂ in normoxic conditions in XVIVO-10 media supplemented with 10 μM RHO/ROCK pathway inhibitor Y-27632.

### AAV Manufacturing

Recombinant rAAVs (comprising a capsid of SEQ ID NO:170), for in vitro studies were produced via PEI-mediated triple transfection into HEK293 cells. Cells were cultured in DMEM supplemented with FBS and maintained at 37° C. in a 5% CO₂ environment.

Briefly, cells were seeded into CellSTACK vessels and transfected with pHelper, the relevant Rep-Cap plasmids, and the CAG-nanobody payload plasmid. Three to four days following transfection, cells and supernatant were collected and lysed, endonuclease treated, clarified via sterile filtration, and frozen. Thawed harvest material was clarified and loaded onto relevant affinity resins, eluted at low pH and immediately neutralized. Purified lots were formulated in either a phosphate or Tris-based buffer system containing NaCl and 0.005% Pluronic F-68. Purified lots were stored at −80° C. until use.

### Transfection of VHH Plasmids

HEK293T cells were seeded in 12-well plates at 2×10⁵ cells/well and transfected with various plasmid versions of C3N10, C3S3 and C5S2. Supernatants were collected and tested for transgene expression by various assays.

### Transduction

iPSC-RPE were transduced ≥30 days after seeding with a multiplicity of infection (MOI, vg/cell) of 1,000, 5,000 or 20,000. Supernatant samples were collected 7 days post infection for analysis.

### VHH ELISA

Anti-VHH capture antibody was diluted 1:500 in PBS and incubated in a MaxiSorp microtiter plate overnight at 4° C. The plate was then washed 5× with PBST and incubated in a blocking solution of 2% BSA in PBS at room temperature for 2 hours. Plate washing was likewise performed between each following step unless otherwise noted. Superatants from transduced cells were diluted in 1% BSA in PBS; dilutions were uniform across all samples of each VHH tested. Diluted samples were applied to the microtiter plate and incubated at room temperature for 2 hours. The plate was next incubated with HRP-conjugated anti-VHH detection antibody diluted 1:10,000 in 1% BSA in PBST for 1 hour at room temperature. Following incubation with detection antibody, 100 μl TMB substrate was applied to the plate and color development was monitored. 100 μl 450 nM TMB stop solution was applied to the plate without a wash step 5-15 minutes after addition of TMB substrate. The optical density (OD) was measured with a Cytation 3 photometer at 450 nm within 15 minutes of applying the stop solution.

### Western Blot

Protein concentrations of cell supernatants were quantified using the Pierce Micro BCA Protein assay according to the manufacturer's instructions. Appropriate volume of supernatant to achieve 33 μg of total protein was then diluted in PBS to 15 μl and mixed with 5.78 μl 4×LDS buffer and 2.31 μl 10× Reducing agent and incubated at 90° C. for 10 minutes. Samples were loaded on a 15-well Bolt 12% Bis-Tris Plus polyacrylamide gel or Novex 10 to 20% Tricine polyacrylamide gels and run in 1×MOPS buffer at 160 volts for 45 minutes. Separated proteins were transferred to a PVDF membrane with the BioRad TransBlot Turbo device (Cat. No. 1704150, Bio-Rad) for 10 minutes at 2.5 A. The membrane was probed with either HRP-conjugated anti-VHH primary antibody, Mouse-anti C3a, Rabbit-anti C5a or Goat anti-Human Factor B with appropriate HRP secondary antibodies if not conjugated, in iBind Flex Solution with an iBind Flex card. Proteins were visualized with SuperSignal West Pico Plus Chemiluminescent Substrate and imaged on a ChemiDoc MP.

### Complement Inhibition Assay for Transduced Sample Assessment

The Wieslab® Complement system Alternative pathway kit is an enzyme immunoassay for the qualitative determination of functional alternative complement pathway in human serum and was executed according to manufacturer's instructions. Normal human serum was diluted to 2% in the provided dilution buffer and 200 μl of the 2% serum was transferred to a microtiter plate. 20 μl supernatant samples from transduced RPE cultures were applied to the plate and mixed. Following mixing, the diluted serum with added supernatant samples was transferred in duplicate to the assay plate, 100 μl per well, and incubated for 60 min at 37° C. Post incubation, the plate was washed 3 times with the wash buffer provided in the kit and incubated with the conjugate for 30 min at room temperature. Following incubation, the plate was washed 3 times with the wash buffer provided. One hundred micro-liter of the substrate solution provided with the kit was added to the microtiter plate and absorbance was read at 405 nm every 5 min intervals for 90 minutes. Percent complement inhibition for each sample was calculated according to the following formula and was considered to be semi-quantitative.

$$\% Inhibition = ( 1 - \frac{Sample OD}{Average non - transduced sample OD} ) \times 100$$

### Results

### Wild Type and Humanized Nanobodies Binding Affinity to C3 and C5 Targets.

Wild type nanobodies targeting C3, C3N10 and C3S3, and C5, C5S2, were humanized as C3N10.3, C3S3.2 and C582.2 respectively. All six were recombinantly expressed from transiently transfected human HEK293 suspension cells, purified, and further tested below. Table 5 contains binding affinities of the wild-type and humanized nanobodies to human and cynomolgus C3 and C5 complement protein targets. The KDs, in nM, were calculated via biolayer interferometry (BLI) using the Gator. Not only do neither C3 nor C5 targeted wild type nanobodies cross-bind to the other antigen, but none of them also associate with other members of the alpha-2-macroglobulin family: human C4b, alpha-2-macroglobulin, and alpha-2-macroglobuline-like-1 protein (data not shown).

TABLE 5
Affinity of C3 and C5 Wild Type and Humanized Nanobodies to
Human and Cynomolgus C3 and C5 protein, (NB: No binding.)

| | KD (nM) | KD (nM) | KD (nM) | KD (nM) |
|---|---|---|---|---|
| Nanobody | Hu C3 | Cy C3 | Hu C5 | Cy C5 |
| C3S3 (SEQ ID NO: 2) | 4.97 | 1.13 | NB | NB |
| C3S3.2 (SEQ ID | 5.83 | 1.77 | NB | NB |
| NO: 98) | | | | |
| C3N10 (SEQ ID NO: 14) | 3.64 | 1.37 | NB | NB |
| C3N10.3 (SEQ ID | 5.32 | 1.82 | NB | NB |
| NO: 135) | | | | |
| C5S2 (SEQ ID NO: 58) | NB | NB | 6.29 | NB |
| C5S2.2 (SEQ ID | NB | NB | 12 | NB |
| NO: 141) | | | | |

### Mechanism of Action: Complement Pathway Inhibitory Activity

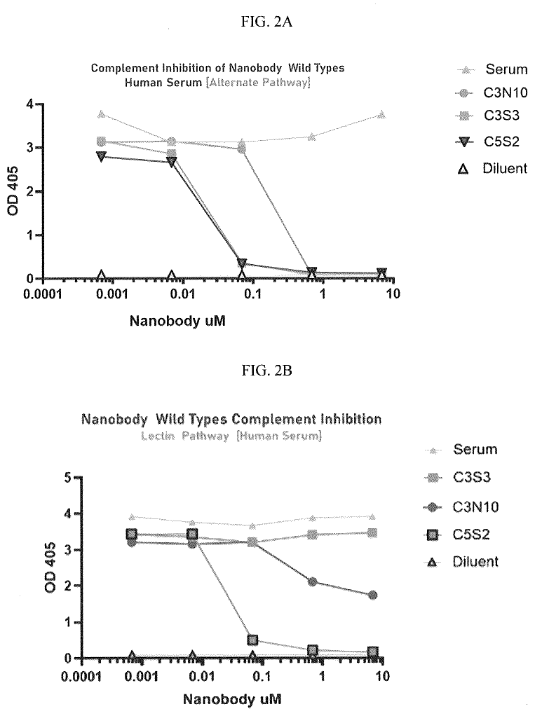

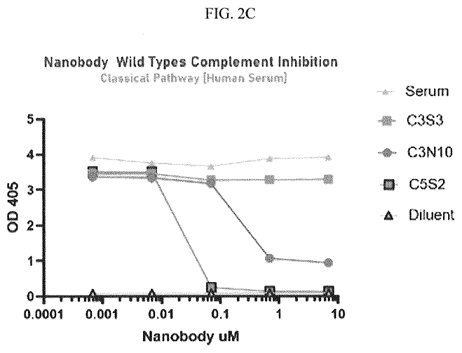

The innate immune complement system is initiated by three separate pathways: the classical, lectin, and alternative pathways. The C3 protein acts as the point of convergence of each pathway's activation, while C5 protein cleavage initiates the formation of the final component of complement activation: the membrane attack complex. To distinguish their mechanism of action within the three pathways of the complement cascade, the wild type nanobodies' functional potential were evaluated in classical, alternative, and classical pathway assay kits in human serum available from Wieslab. As shown in FIG. 2, all three nanobodies have inhibitory activity against the alternative pathway. However, while C5S2 has strong inhibitory activity in all three pathways, C3S3 only has activity against the alternative pathway. C3N10 shows most inhibitory activity against the alternative pathway and moderate potency against both the classical and lectin pathways.

### IC50 Potency of the Wild Type and Humanized Nanobodies Against Human Serum Mediated Alternative Pathway.

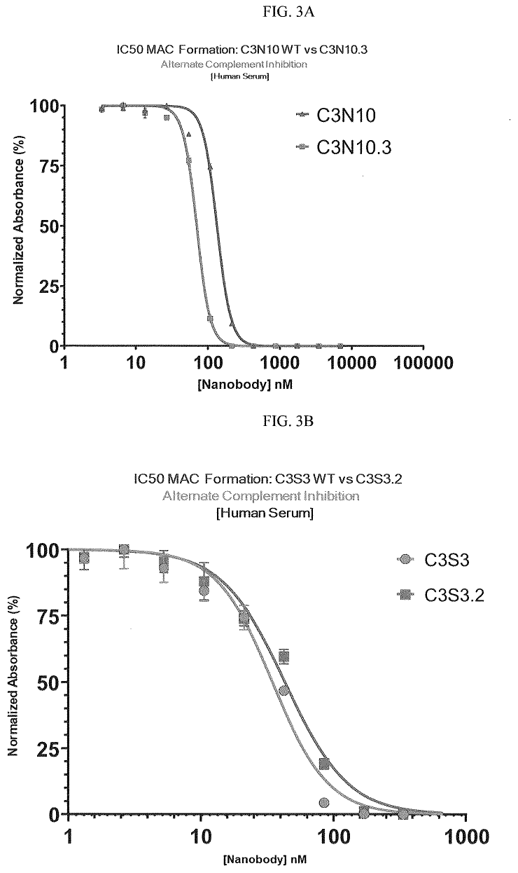

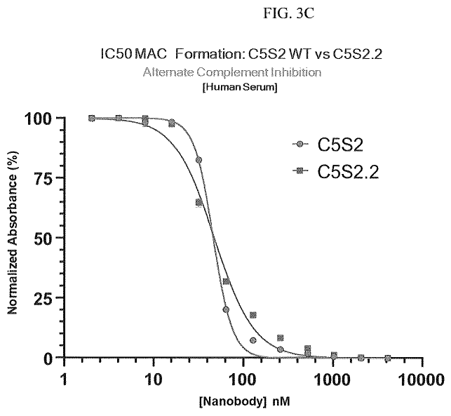

To calculate IC50s and compare potency between wild type and humanized versions of the nanobodies, all were serially diluted 1:2 and re-tested in same alternative pathway assay. Humanized nanobodies have similar potency to their original wild type counterparts, implicating little loss in inhibitory activity between variants. FIGS. 3A-C illustrates their potency, while Table 6 summarizes these results. The IC₅₀ of C3N10 (wild type) was 133.8 nM; the IC₅₀ of C3N10.3 was 69.47 nM (FIG. 3A). The IC₅₀ of C3S3 (wild type) was 34.51 nM; the IC50 of C3S3.2 was 42.86 nM (FIG. 3B). The IC₅₀ of C5S2 (wild type) was 46.18 nM; the IC50 of C5S2.2 was 47.33 nM (FIG. 3C)

### IC50 of C3N10 WT and Humanized in Classical and Lectin Pathways

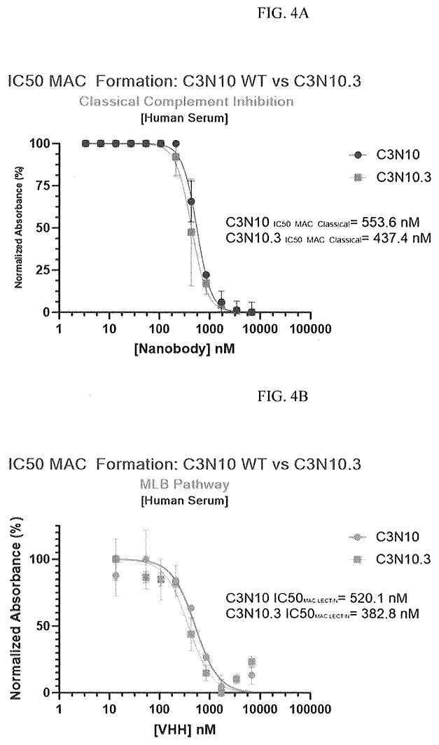

Because C3N10 showed mild inhibition in both classical and lectin pathways, the wild type and humanized C3N10.3 IC50s were calculated for both pathways with more serial dilutions as shown in FIGS. 4A-B and summarized in Table 6 below.

TABLE 6
Summary of wild-type and humanized nanobodies complement pathways
IC 50 inhibition potency. (NA: No activity; ND: Not done)

| | IC50 Alternative | IC50 Classical | IC50 Lectin |
|---|---|---|---|
| Nanobody | (nM) | (nM) | (nM) |
| C3S3 | 34.51 | NA | NA |
| C3S3.2 | 42.86 | NA | NA |
| C3N10 | 133.8 | 554 | 524 |
| C3N10.3 | 69.47 | 473 | 421 |
| C5S2 | 46.18 | ND | ND |
| C5S2.2 | 47.33 | 46 | 42 |

### Anti-Human C3 and C5 Wild Type Nanobody Activity Against Mouse Alternative Pathway.

To examine whether the human alternative complement pathway inhibiting nanobodies are cross functional against mice, the wild type versions were tested using Hycult's Mouse C3/C5 Elisa kit (Catalog #HK2002). The positive control mouse-anti-mouse C5 inhibiting antibody BB5.1 was included. As shown in FIG. 5, C3N10, C3S3 and C5S2 had no observable inhibitory activity in this mouse serum mediated alternative complement pathway assay.

### IC50 of Anti-Human C3 and C5 Wild Type and Humanized Nanobodies Against Alternative Complement Activity in Non-Human Primate Serum

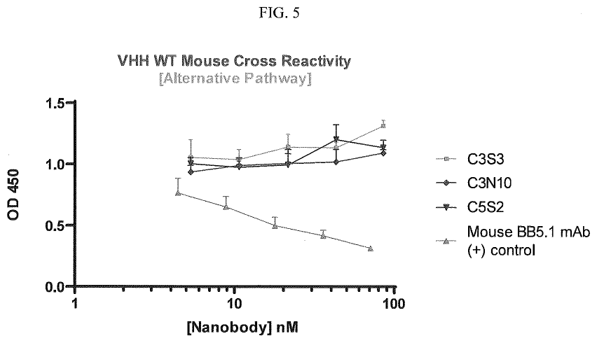

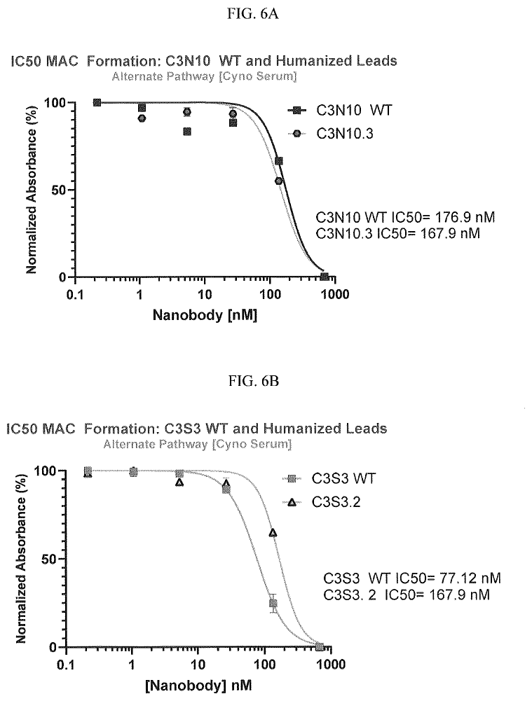

Based upon their binding affinity to their respective cynomolgus variants, the IC50 of the wild type and humanized nanobodies was evaluated in the same Wieslab alternative complement pathway assay using cynomolgus serum, as shown in FIGS. 6A and 6B and Table 7 below. While both C3 targeting wild type and humanized nanobodies showed inhibitory activity, neither C5S2 nor its humanized version exhibited Wieslab cynomolgus serum mediated alternative complement pathway potency (data not shown), reflecting their inability to bind to their cynomolgus C5 protein target in Table 5.

TABLE 7
IC50 of C3 and C5 Nanobodies Alternate
Pathway Primate Serum (NA: no activity)

| C3N10 WT | C3N10.3 | C3S3 WT | C3S3.2 | C5S2 WT | C5S2.2 |
|---|---|---|---|---|---|
| 176.9 nM | 167.9 nM | 77.12 nM | 167.9 nM | NA | NA |

### Binding Rate and Alternative Complement Pathway Inhibition by Humanized Nanobody-Human Fc Fusion Proteins

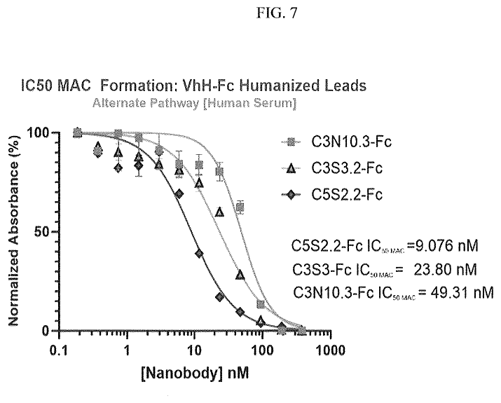

The three humanized nanobodies, C3N10.3, C3S3.2, and C5S2.2, were engineered and expressed as homo-dimeric fusion proteins (aka, C3N10.3-Fc, C3S3.2-Fc and C5S2.2-Fc) with the human G2 hinge-G4 Fc domain to emulate native immunoglobulin bivalent structure and function. Their respective binding rate to target and alternative pathway inhibition potency was calculated using biolayer interferometry and the Wieslab assays and compared to the original monomers in Table 8 and 9, respectively. Avidity association was observed with C3S3.2-Fc and C5S2.2-Fc while synergistic alternative pathway inhibitory activity (greater than two-fold) appears to be seen with C5S2-Fc (FIG. 7).

TABLE 8
Binding KD of humanized monomer
and Fc fusion proteins to target

| | | | |
|---|---|---|---|
| Format | C3N10.3 | C3S3.2 | C5S2.2 |

| | | | | | | |
|---|---|---|---|---|---|---|
| Monomer | 5 | nM | 6 | nM | 12 | nM |
| Fc fusion | 3.25 | nM | 0.703 | nM | 0.654 | nM |

TABLE 9
Inhibition potency IC50 against alternative complement pathway

| | | | |
|---|---|---|---|
| Format | C3N10.3 | C3S3.2 | C5S2.2 |

| | | | | | | |
|---|---|---|---|---|---|---|
| Monomer | 69.47 | nM | 42.86 | nM | 47.33 | nM |
| Fc fusion | 49.31 | nM | 23.08 | nM | 9.076 | nM |

### Epitope Peptide Map of C3N10.3 and C3S3.2 to Human C3 Protein

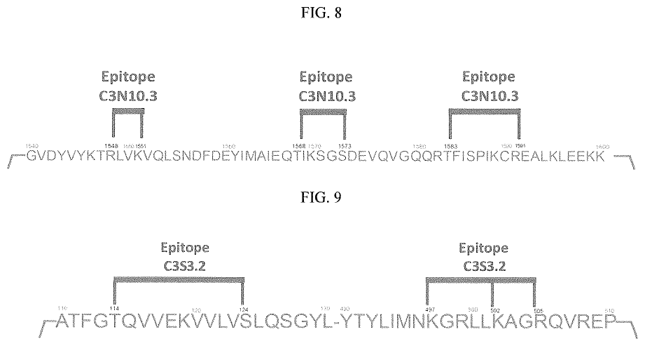

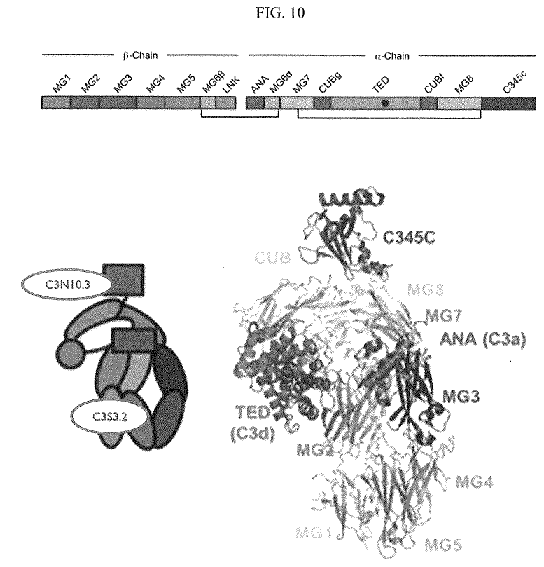

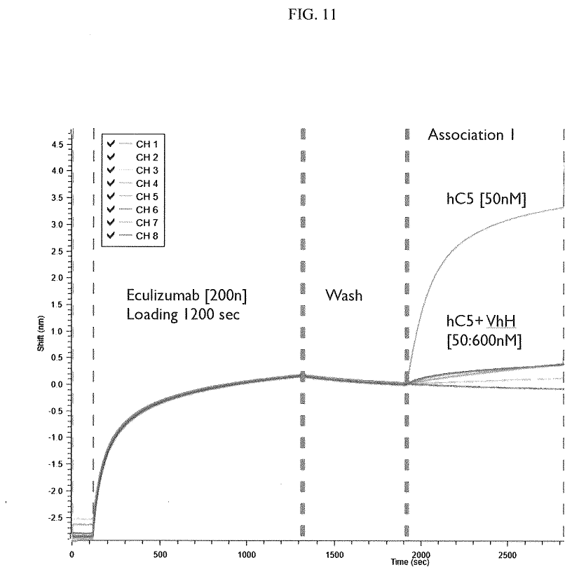

To further distinguish the mechanism of action between the two C3 targeting VHHs, cross-linking mass spectrometry (XL-MS, CovalX) was used to identify C3N10.3 and C3S3.2 binding epitope. Both humanized nanobodies and purified C3 human protein were provided. Tables of cross-linked peptides between the nanobody and antigen along with illustrated amino-acid interactions are shown in Table 10 and FIG. 8 for C3N10.3 and Table 11 and FIG. 9 for C3S3.2, respectively. Based upon their interacting residues, CN10.3's binding epitope is within C3's alpha-chain at the C345C domain (1540-1600: GVDYVYKTRLVKVQLSNDFDEYIMAIEQTIKSGSDEVQVGQQRTFISPIKCREALKLEEK K (SEQ ID NO:171)), while C3S3.2 appears to associate with two non-linear epitopes in the beta-chain across the MG1 (110-130: ATFGTQVVEKVVLVSLOSGYL (SEQ ID NO:172) and MG5 (490-510: YTYLIMNKGRLLKAGRQVREP (SEQ ID NO:173) domains, FIG. 10.

TABLE 10
Cross-linked peptides detected between C3N10.3 and human C3

| | | | | Protein | Protein | | | |
|---|---|---|---|---|---|---|---|---|
| Sequence | Enzyme | Protein1 | Protein2 | Position 1 | Position 2 | XLType | nAA1 | Naa2 |
| NTLYLQMINSLRPEDTAVY | Trypsin | C3W10.3 | hc3 | 77-103 | 1547-1570 | inter- | 102 | 1548 |
| SCAAAPLTR-TALVKVQLS | | | | | | protein | | |
| NQFDEYIMAIEQTIK-a26- | | | | | | xl | | |
| b2 | | | | | | | | |
| GNPYYADS-KVQLS-a4-51 | Elastase | C3W10.3 | hc3 | 56-63 | 1551-1555 | inter- | 59 | 1551 |
| | | | | | | protein | | |
| | | | | | | xl | | |
| GLTFSPYA-MAIEQTI-a7- | Elastase | C3W10.3 | hc3 | 26-33 | 1563-1569 | inter- | 32 | 1568 |
| b6 | | | | | | protein | | |
| | | | | | | xl | | |
| TRSYDYVWG-IEQTI- | Elastase | C3W10.3 | hc3 | 102-109 | 1565-1569 | inter- | 105 | 1568 |
| a4-b4 | | | | | | protein | | |
| | | | | | | xl | | |
| TFSPY-AIEQTIKSGSDE | Chymotrypsin | C3W10.3 | hc3 | 28-32 | 1562-1584 | inter- | 30 | 1573 |
| VQVGQQRTF-a3-b12 | | | | | | protein | | |
| | | | | | | xl | | |
| GNPYYA-QQRTFISPI- | Elastase | C3W10.3 | hc3 | 56-61 | 1580-1588 | inter- | 59 | 1583 |
| a4-b4 | | | | | | protein | | |
| | | | | | | xl | | |
| LTRSYDYWGQGTLVT- | Thermolysin | C3W10.3 | hc3 | 101-115 | 1588-1592 | inter- | 107 | 1591 |
| IKCRE-a7-b6 | | | | | | protein | | |
| | | | | | | xl | | |
| SGNPYYADSVKGR- | Thermolysin | C3W10.3 | hc3 | 54-67 | 1588-1595 | inter- | 59 | 1591 |
| IKCREALK-a6-b4 | | | | | | protein | | |
| | | | | | | xl | | |
| LTRSYDYWGQGT- | Thermolysin | C3W10.3 | hc3 | 101-112 | 1588-1595 | inter- | 105 | 1591 |
| IKCREALK-a5-b4 | | | | | | protein | | |
| | | | | | | xl | | |
| EVQLVESGGGLVQAG | Trypsin | C3W10.3 | hc3 | 1-3 [symbol] | 1590-1595 | inter- | 30 | 1591 |
| GSERLACAASGLIFS | | | | | | protein | | |
| PYAMGWER-CREALK- | | | | | | xl | | |
| a30-b2 | | | | | | | | |

[symbol]

indicates data missing or illegible when filed

TABLE 11
Cross-linked peptides detected between C3S3.2 and human C3

| | | | | Protein | Protein | | | |
|---|---|---|---|---|---|---|---|---|
| | | | | Posi- | Posi- | | | |
| | | | | tion | tion 2 | | | |
| Sequence | Enzyme | Protein1 | Protein2 | 1 | | XLType | nAA1 | nAA2 |
| CAAVPTYRPY | Chymotrypsin | C353.2 | hc3 | 95-104 | 106-122 | Inter- | 101 | 114 |
| -VTVQATFGT | | | | | | protein | | |
| QVVEKVVL-a | | | | | | xl | | |
| 7-b9 | | | | | | | | |
| SCAASGTIF- | Chymotrypsin | C353.2 | hC3 | 21-29 | 106-125 | inter- | 25 | 114 |
| VTVQATFGTQ | | | | | | protein | | |
| VVEKVVLVSL | | | | | | xl | | |
| -a5-b9 | | | | | | | | |
| SCAASGTIF- | Chymotrypsin | C353,2 | MC3 | 21-29 | 106-125 | Inter- | 25 | 124 |
| VTVQATFGTQ | | | | | | protein | | |
| VVEKVVLVSL | | | | | | xl | | |
| -a5-b19 | | | | | | | | |
| SRNYMGWY-L | Chymotrypsin | C353.2 | HC3 | 30-37 | 493-501 | inter- | 33 | 497 |
| IMNKGRLL-a | | | | | | protein | | |
| 4-65 | | | | | | xl | | |
| LSCAASGTIF | Trypsin | C353.2 | WC3 | 20-38 | 500-505 | inter- | 30 | 902 |
| SRNYMGWYR- | | | | | | protein | | |
| ILKAGR-a11 | | | | | | xl | | |
| -b3 | | | | | | | | |
| AVPTYRPYYF | Elastase | C353.2 | AC3 | 97-111 | 499-503 | inter- | 104 | 502 |
| YWGQG-RLLK | | | | | | protein | | |
| A-a8-b4 | | | | | | xl | | |
| AVPTYRPYYF | Elastase | C353.2 | HC3 | 97-109 | 505-511 | inter- | 102 | 505 |
| YWG-RQVREP | | | | | | protein | | |
| G-a6-b1 | | | | | | xl | | |

### Epitope Interrogation of C5S2.2: Cross-Blocking of Eculizumab to Human C5 Protein

Clinically approved eculizumab, like C5S2, binds to human C5 protein, inhibits all three complement pathways in human serum, and neither binds to cynomolgus C5 nor inhibits complement pathways in cynomolgus serum (data not shown). The Gator was used to investigate whether eculizumab and C5S2 also cross-block each other to human C5.

In FIG. 11, the anti-human Fc-binding tips were first loaded with eculizumab then washed. These pre-loaded tips were then dipped into untagged C5 alone or C5 preincubated with C5S2 in the recommended binding buffer. C5 protein alone bound rapidly to the eculizumab pre-loaded tips as expected while the C5 preincubated with C5S2 did not, indicating the latter pre-bound nanobody was blocking the former full-size antibody's epitope.

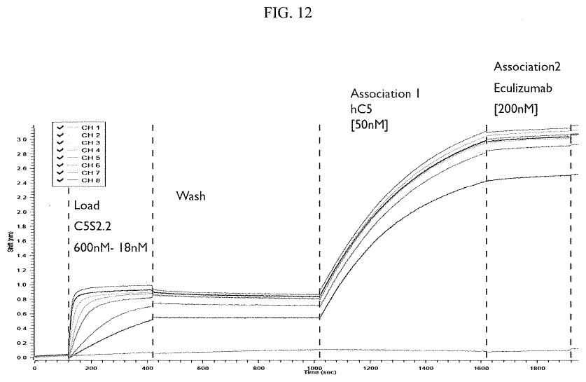

In an alternative experiment in FIG. 12, the C5S2 nanobody was first bound to the strep-II tips. After washing, this pre-loaded tip was dipped into C5 containing binding solution and rapid association was observed, as expected. However, when this pre-bound C5S2-C5 complex tip was dipped into eculizumab containing binding solution, the latter failed to bind, indicating that its binding site was blocked.

Epitope Interrogation of C5S2.2: Compare with Eculizumab's Binding to Mutant C5 Proteins

Eculizumab is refractory to two naturally occurring variants: the R885H human polymorphism and the W917S human vs cynomolgus divergence (Brachet G et. al. 2016; Nishimura J et. al. 2014). Human C5 R885H, human C5 W917S, and cynomolgus C5 S917W mutant proteins were recombinantly produced and C5S2.2's and eculizumab's affinity to them was compared using the Gator.

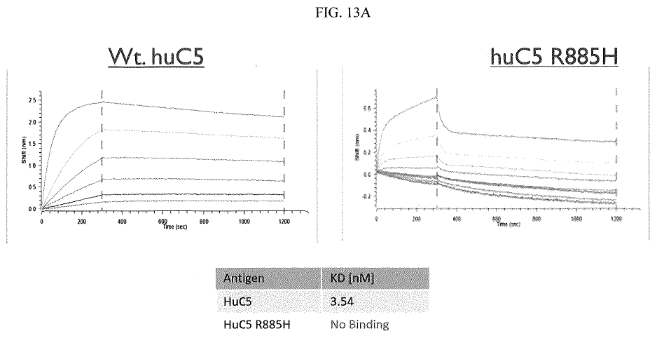

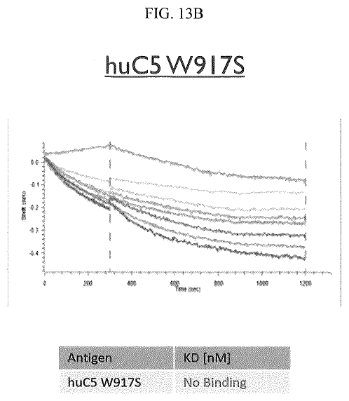

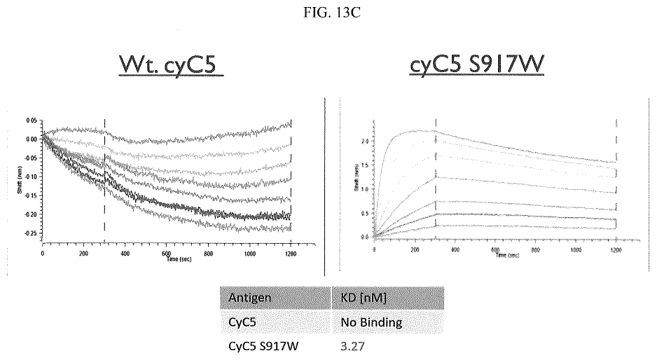

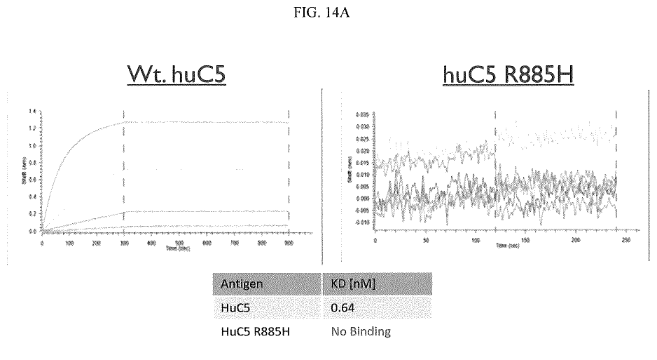

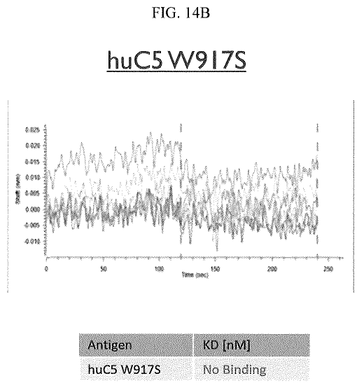

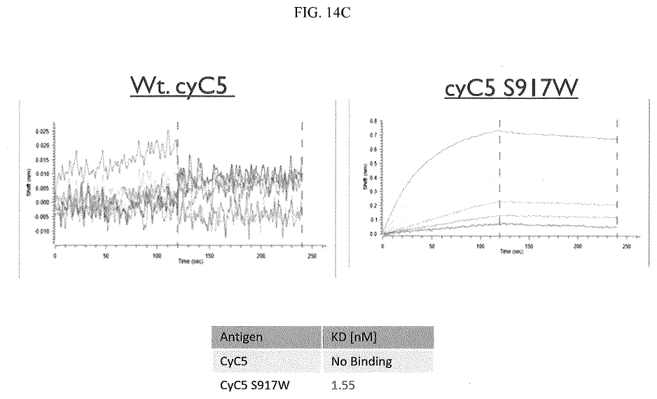

FIGS. 13A-C show, while C5S2.2 had high affinity for the wild-type human C5 protein, it lost all binding activity to human C5 R885H and W917S mutants. On the other hand, while it was unable to bind to wild type cynomolgus C5, C5S2.2 had high affinity to the mutant cynomolgus C5 S917W. This pattern of interaction to wild-type vs mutant was reflected by eculizumab, as expected, in FIGS. 14A-C, indicating that C5S2.2 and eculizumab have similar binding epitopes on human C5.

Anti-Drug Antibody Response from Healthy Donor's Serum Against Wild Type and Humanized Nanobodies

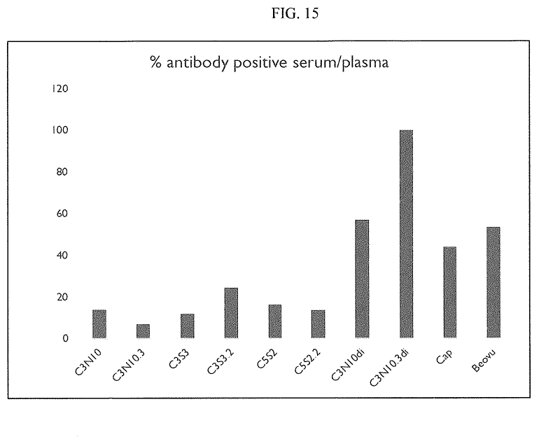

To examine the relative potential immunogenicity of the wild-type and humanized nanobodies, a sandwich immunoassay on MSD with passively coated antigen and anti-human IgG detector was used to measure the seroprevalence of pre-existing antigen-specific antibodies in serum samples collected from individual healthy human donors. The percentage of donors that had detectable pre-existing antibodies to each antigen is plotted in both table 12 and graph form in FIG. 15. In comparing different antigens, pre-existing antibodies to monomeric C3/5 nanobodies had a lower seroprevalence in human serum samples than against (G4S)2-linked homodimers, C3N10 Di and C3N10.3 Di. The percentage of human samples with pre-existing antibodies against clinically approved Beovu and Cablivi are shown as comparison controls. Beovu is a humanized monoclonal single-chain variable fragment (scFv) that binds and inhibits vascular endothelial growth factor A while Cablivi (caplacizumab) is a tri-alanine-linked homo-dimeric nanobody against the A1 domain of von Willebrand factor.

TABLE 12

| Antigen | Ab Positive |
|---|---|
| C3N10 | 6/43, 14% |
| C3N10.3 | 5/44, 11% |
| C3S3 | 5/42, 12% |
| C3S3.2 | 10/41, 24% |
| C5S2 | 7/43, 16% |
| C5S2.2 | 6/44, 14% |
| C3N10 Di | 8/14, 57% |
| C3N10.3 Di | 15/15, 100% |
| Cap | 18/41, 44% |
| Beovu | 22/41, 44% |

### AAV Carrying VHH DNA Lead to Functional Protein Production in Human Cells

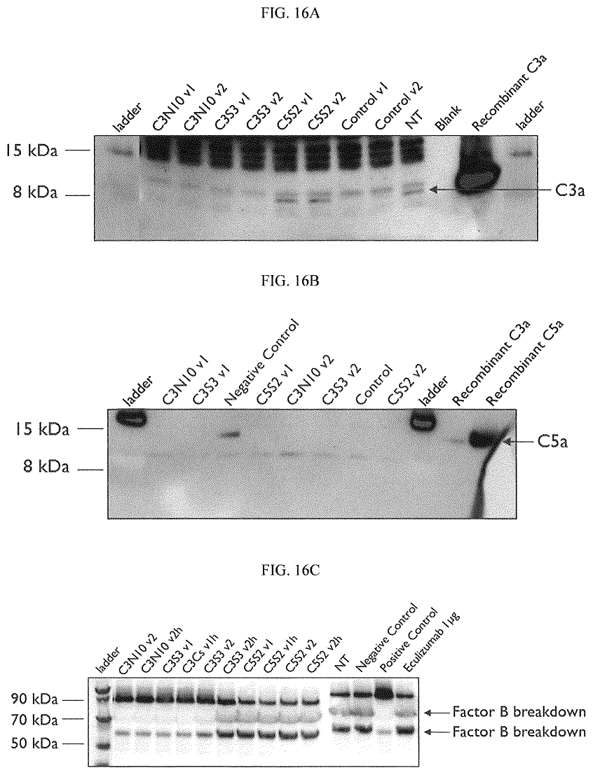

Different versions of lead VHHs, C3N10, C3S3 and C5S2 were cloned into AAV supportive backbones in preparation for AAV manufacturing. Prior to AAV manufacturing VHH containing plasmids were assessed for function via western blot assays. As depicted in FIG. 16, upon activation of the alternative complement cascade C3 gets broken down into C3a and C3b. C3 breakdown triggers activation of C5 degradation resulting in C5a and C5b. A feedback loop of C3 activation can be initiated by this activation and result in Factor B breakdown. C3 and C5 VHH inhibitors can be assessed for function to stop these degradation products, which can be visualized by western blot.

Supernatants from transfected cells were subjected to western blot to examine breakdown products indicative of an activated complement cascade pathway (FIGS. 16A-C). All plasmids containing a C3 VHH inhibitor blocked formation of the degradation product C3a, whereas inhibitors to C5, downstream of C3, did not, as expected. All VHH inhibitors to C3 and C5 blocked degradation of C5a. Factor B breakdown products were next assessed, and all C3 VHH inhibitors blocked breakdown, however C5 VHH inhibitors, acting further downstream, did not.

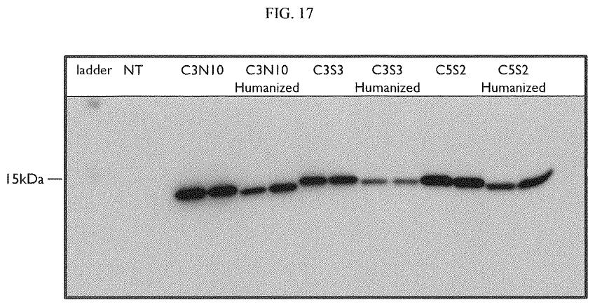

Following plasmid assessment, VHH within the AAV supportive plasmid backbone were manufactured into AAV. Lead VHH AAV were characterized by expression and activity following transduction at three MOI in iPSC-RPE. FIG. 17 depicts a Western blot of the supernatant collected post transduction, showing a single product at the expected size of 15 kDa for each VHH examined (FIG. 17).

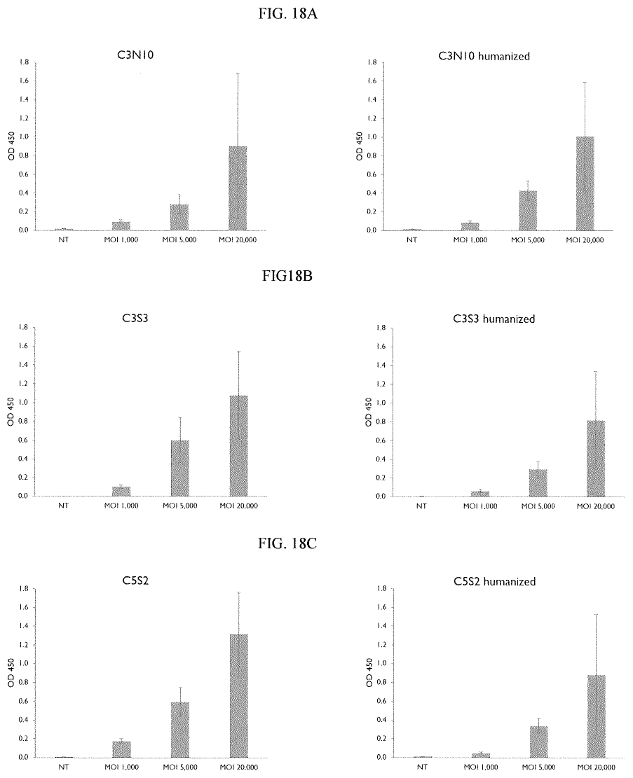

VHH expression in the supernatant was also determined by ELISA (FIG. 18). Each VHH exhibited a dose response as indicated by an increase in OD at 450 nm with increasing dose.

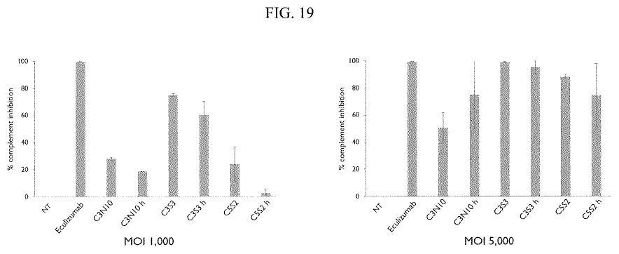

Next, the function of each VHH was assessed by determining complement pathway inhibition using the Wieslab® assay (FIG. 19). At both MOI tested, complement inhibition was detected, although at varying degrees for each specific VHH.

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention.

1. An isolated polypeptide comprising a VH or VHH domain that specifically binds to complement factor C3 and/or proteolytic derivatives C3a and/or C3b, preferably C3, wherein said VH or VHH domain comprises one or more complementarity-determining regions (CDRs) at least 75% identical to a CDR selected from the group consisting of SEQ ID Nos: 18-20, 54-56, 15-17, and 21-53.

2. The isolated polypeptide according to claim 1, wherein the CDRs of the VH or VHH domain consist of CDR1 selected from SEQ ID Nos: 18, 54, 15 21, 24, 27, 30, 33, 36, 39, 42, 45, 48, 51, and a sequence having at least one, at least two or at least three amino acid differences relative to SEQ ID Nos: 18, 54, 15 21, 24, 27, 30, 33, 36, 39, 42, 45, 48, or 51; CDR2 selected from SEQ ID Nos: 19, 55, 16 22, 25, 28, 31, 34, 37, 40, 43, 46, 49, 52, and a sequence having at least one, at least two or at least three amino acid differences relative to SEQ ID Nos: 19, 55, 16 22, 25, 28, 31, 34, 37, 40, 43, 46, 49, or 52; and CDR3 selected from SEQ ID NOs: 20, 56, 17 23, 26, 29, 32, 35, 38, 41, 44, 47, 50, 53, and a sequence having at least one, at least two or at least three amino acid differences relative to SEQ ID NOs: 20, 56, 17 23, 26, 29, 32, 35, 38, 41, 44, 47, 50, or 53.

3. The isolated polypeptide according to claim 1 or 2, wherein the CDRs are separated by framework regions (FR) FR1, FR2, FR3 and FR4.

4. The isolated polypeptide according to any one of claims 1-3, wherein the VH or VHH domain comprises the CDR sequences of any one of the VH or VHH domains set forth as SEQ ID Nos: 2, 14, 1 and 3-13 or CDR sequences at least 75% identical thereto, preferably wherein the VH or VHH domain comprises a CDR1 having the following sequence: G-F/L-T/S-F/L-S/G/D-P/V/Y/N-Y-A/D-M/I-G/S/A, and/or comprises a CDR2 having the following sequence: G/S/C-I-R/D/T/S/N-W/S/G/Q-I/G/P/S-S/V/D/R-G/N-N/T/S-P/A/T/Y-Y/H/A-Y/H, wherein the 11^{th} amino acid is optional.

5. The isolated polypeptide according to claim 4, wherein the VHH domain comprises the amino acid sequence set forth as any one of SEQ ID Nos: 2, 14, 1 and 3-13 or an amino acid sequence at least 75% identical thereto.

6. The isolated polypeptide according to any one of claims 1-4, wherein the VH or VHH domain is a humanized VH or VHH domain.

7. The isolated polypeptide according to claim 6, wherein the VH or VHH domain comprises the amino acid sequence of any one of SEQ ID Nos: 98, 135, 94-97, 99-134 and 136 or comprises an amino acid sequence at least 75% identical thereto, preferably, wherein the VH or VHH domain comprises the amino acid sequence of any one of SEQ ID Nos: 98, 135, 94-97, 99-134 and 136 or comprises an amino acid sequence at least 75% identical thereto.

8. The isolated polypeptide according to any one of claims 1-7, wherein the VH or VHH domain is coupled to a tag, preferably a Strep or his6 tag, more preferably wherein the his6 tag comprises or consists of the amino acid sequence set forth as SEQ ID NO:93, and is at the C-terminal of the VH or VHH domain.

9. A nucleic acid comprising a nucleotide sequence encoding a polypeptide according to any one of claims 1-8.

10. A recombinant adeno-associated virus (rAAV) comprising (i) a variant AAV capsid protein comprising a heterologous peptide with a length of 7, 8, 9, 10 or 11 amino acids covalently inserted in the GH-loop of the capsid protein relative to a corresponding parental AAV capsid protein, wherein the peptide insertion comprises the amino acid sequence ISDQTKH (SEQ ID NO:168) and (ii) a nucleic acid according to claim 9.

11. The rAAV according to claim 10, wherein the insertion peptide has from 1 to 3 spacer amino acids (Y₁-Y₃) at the amino and/or carboxyl terminus of amino acid sequence ISDQTKH (SEQ ID NO:168), preferably wherein the insertion peptide is LAISDQTKHA (SEQ ID NO:169).

12. The rAAV according to claim 10 or 11, wherein the insertion site is located between amino acids corresponding to amino acids 587 and 588 of VP1 of AAV2 (SEQ ID NO: 183) or the corresponding position in the capsid protein of another AAV serotype.

13. The rAAV according to any one of claims 10 to 12, wherein the capsid protein comprises one or more amino acid substitution(s) relative to VP1 of AAV2 (SEQ ID NO:183) or one or more corresponding substitution(s) in the capsid protein of another AAV serotype, preferably one or more of the following amino acid substitutions: MIL, L15P, P34A, N57D, N66K, R81Q, Q101R, S109T, R144K, R144M, Q164K, T176P, L188I, S196Y, G226E, G236V, I240T, P250S, N312K, P363L, D368H, N449D, T456K, S463Y, D472N, R484C, A524T, P535S, N551S, A593E, 1698V, V708I, V719M, S721L, and L735Q, more preferably a P34A amino acid substitution.

14. The rAAV according to claim 13, wherein the capsid protein comprises a P34A amino acid substitution relative to VP1 of AAV2 and comprises an amino acid sequence at least 90% identical, at least 95% identical, at least 98% identical or 100% identical to the entire length of the amino acid sequence set forth as SEQ ID NO:170, preferably wherein the capsid protein consists of the amino acid sequence set forth as SEQ ID NO:170.

15. The rAAV according to any one of claims 10 to 14, wherein the rAAV exhibits an increased infectivity, preferably at least a 2-fold increased infectivity, of a retinal cell compared to the infectivity of the retinal cell by an AAV comprising the corresponding parental AAV capsid protein.

16. The rAAV according to any one of claims 10 to 15, wherein the variant AAV capsid protein comprises an amino acid sequence with 100% sequence identity to the amino acid sequence set forth in SEQ ID NO:170.

17. The rAAV according to any one of claims 10 to 16, wherein the rAAV comprises a heterologous acid comprising from 5′ to 3′: (a) an inverted terminal repeat (b) a promoter (c) a nucleotide sequence encoding a polypeptide according to any one of claims 1-8 (d) a polyadenylation sequence and/or a WPRE sequence and (e) an inverted terminal repeat.

18. The rAAV according claim 17, wherein the rAAV wherein the inverted terminal repeat is an AAV2 inverted terminal repeat.

19. The rAAV according to any one of claims 10 to 18, wherein the promoter is a ubiquitous promoter.

20. The rAAV according to claim 19, wherein the promoter is a CAG promoter

21. The rAAV according to any one of claims 10 to 18, wherein the promoter is a tissue-specific promoter.

22. A host cell comprising the rAAV according to any one of claims 10 to 21.

23. A pharmaceutical composition comprising the rAAV according to any one of claims 10 to 21 and a pharmaceutically acceptable carrier, diluent, excipient or buffer.

24. A method for treating dry age-related macular disorder (dry AMD) in a subject in need thereof, comprising is administering to the subject a therapeutically effective amount of the rAAV according to any one of claims 10-21 or the pharmaceutical composition according to claim 23.

25. The method according to claim 24, wherein the rAAV or the pharmaceutical composition is administered to the subject by periocular, intravitreal, suprachoroidal or subretinal administration at a dosage from about 10⁸ vector genomes (vg)/eye to about 10¹³ vg/eye, or preferably dosage from about 6×10⁹ vg/eye to about 6×10¹⁰ vg/eye.

26. The method according to claim 25, wherein the rAAV or the pharmaceutical composition is administered to the subject by intravitreal administration at a dosage from about 1×10⁹ vg/eye to about 1×10¹⁰ vg/eye.

27. A method for treating geographic atrophy (GA) in a subject in need thereof, comprising administering to the subject a therapeutically effective amount of the rAAV according to any one of claims 10 to 21 or the pharmaceutical composition according to claim 23.

28. The method according to claim 27, wherein the rAAV or the pharmaceutical composition is administered to the subject by periocular, intravitreal, suprachoroidal or subretinal administration at a dosage from about 10⁸ vector genomes (vg)/eye to about 10¹³ vg/eye, or preferably dosage from about 6×10⁹ vg/eye to about 6×10¹⁰ vg/eye.

29. The method according to claim 28, wherein the rAAV or the pharmaceutical composition is administered to the subject by intravitreal administration at a dosage from about 1×10⁹ vg/eye to about 1×10¹⁰ vg/eye.

30. A method for delivering the rAAV according to any one of claims 10 to 21 or the pharmaceutical composition according to claim 23 to an eye of a subject, wherein is wherein the rAAV or the pharmaceutical composition is administered to the subject by periocular, intravitreal, suprachoroidal or subretinal administration.

31. The method according to claim 30, wherein the rAAV or the pharmaceutical composition administered to the eye of the subject by intravitreal administration.

32. An isolated polypeptide comprising a VH or VHH domain that specifically binds to complement factor C5 and/or proteolytic derivatives C5a and/or C5b, preferably C5, wherein said VH or VHH domain comprises one or more complementarity-determining regions (CDRs) at least 75% identical to a CDR selected from the group consisting of SEQ ID Nos: 69-71, 66-68 and 72-92.

33. The isolated polypeptide according to claim 32, wherein the CDRs of the VH or VHH domain consist of CDR1 selected from SEQ ID Nos: 69, 66, 72, 75, 78, 81, 84, 87, 90 and a sequence having at least one, at least two or at least three amino acid differences relative to SEQ ID Nos: 69, 66, 72, 75, 78, 81, 84, 87, or 90; CDR2 selected from SEQ ID Nos: 70, 67, 73, 76, 79, 82, 85, 88, 91, and a sequence having at least one, at least two or at least three amino acid differences relative to SEQ ID Nos: 70, 67, 73, 76, 79, 82, 85, 88, or 91; and CDR3 selected from SEQ ID NOs: 71, 68, 74, 77, 80, 83, 86, 89, 92, and a sequence having at least one, at least two or at least three amino acid differences relative to SEQ ID NOs: 71, 68, 74, 77, 80, 83, 86, 89, 92.

34. The isolated polypeptide according to claim 32 or 33, wherein the CDRs are separated by framework regions (FR) FR1, FR2, FR3 and FR4.

35. The isolated polypeptide according to any one of claims 32-34, wherein the VH or VHH domain comprises the CDR sequences of any one of the VH or VHH domains set forth as SEQ ID Nos: 58, 57, and 59-65 or CDR sequences at least 75% identical thereto, preferably wherein the VH or VHH domain comprises a CDR1 having a sequence selected from: G-Y/N/S/T-I-S/F-Y/H/F/A/P/D-D/Q/A/Y/S/E-D/S/T/E/Y/W-D/K/G-M-G and G-Y/N/S/T-I-F-Y/H/F/A/P/D-D/Q/A/Y/S/E-D/S/T/E/Y/W-D-M-G; and/or comprises a CDR2 having a sequence selected from: A/T/G-I-D/T/N-V/Y/G/D-G-G/T/A-S/N/I-T-Y/N and A/T-I-D-V/Y/G/D-G-A-S/N-T-Y.

36. The isolated polypeptide according to claim 35, wherein the VH or VHH domain comprises the amino acid sequence set forth as any one of SEQ ID Nos: 58, 57, and 59-65 or an amino acid sequence at least 75% identical thereto.

37. The isolated polypeptide according to any one of claims 32-35, wherein the VH or VHH domain is a humanized VH or VHH domain.

38. The isolated polypeptide according to claim 37, wherein the VH or VHH domain comprises the amino acid sequence of any one of SEQ ID Nos: 141, 137-140 and 142-163 or comprises an amino acid sequence at least 75% identical thereto.

39. The isolated polypeptide according to any one of claims 32-38, wherein the VH or VHH domain is coupled to a tag, preferably a Strep or his6 tag, more preferably wherein the his6 tag comprises or consists of the amino acid sequence set forth as SEQ ID NO:93, and is at the C-terminal of the VH or VHH domain.

40. A nucleic acid comprising a nucleotide sequence encoding a polypeptide according to any one of claims 32-39.

41. A recombinant adeno-associated virus (rAAV) comprising (i) a variant AAV capsid protein comprising a heterologous peptide with a length of 7, 8, 9, 10 or 11 amino acids covalently inserted in the GH-loop of the capsid protein relative to a corresponding parental AAV capsid protein, wherein the peptide insertion comprises the amino acid sequence ISDQTKH (SEQ ID NO:168) and (ii) a nucleic acid according to claim 40.

42. The rAAV according to claim 41, wherein the insertion peptide has from 1 to 3 spacer amino acids (Y₁-Y₃) at the amino and/or carboxyl terminus of amino acid sequence ISDQTKH (SEQ ID NO:168), preferably wherein the insertion peptide is LAISDQTKHA (SEQ ID NO:169).

43. The rAAV according to claim 41 or 42, wherein the insertion site is located between amino acids corresponding to amino acids 587 and 588 of VP1 of AAV2 (SEQ ID NO: 183) or the corresponding position in the capsid protein of another AAV serotype.

44. The rAAV according to any one of claims 41 to 43, wherein the capsid protein comprises one or more amino acid substitution(s) relative to VP1 of AAV2 (SEQ ID NO:183) or one or more corresponding substitution(s) in the capsid protein of another AAV serotype, preferably one or more of the following amino acid substitutions: MIL, L15P, P34A, N57D, N66K, R81Q, Q101R, S109T, R144K, R144M, Q164K, T176P, L188I, S196Y, G226E, G236V, I240T, P250S, N312K, P363L, D368H, N449D, T456K, S463Y, D472N, R484C, A524T, P535S, N551S, A593E, 1698V, V708I, V719M, S721L, and L735Q, more preferably a P34A amino acid substitution.

45. The rAAV according to claim 44, wherein the capsid protein comprises a P34A amino acid substitution relative to VP1 of AAV2 and comprises an amino acid sequence at least 90% identical, at least 95% identical, at least 98% identical or 100% identical to the entire length of the amino acid sequence set forth as SEQ ID NO:170, preferably wherein the capsid protein consists of the amino acid sequence set forth as SEQ ID NO:170.

46. The rAAV according to any one of claims 41 to 45, wherein the rAAV exhibits an increased infectivity, preferably at least a 2-fold increased infectivity, of a retinal cell compared to the infectivity of the retinal cell by an AAV comprising the corresponding parental AAV capsid protein.

47. The rAAV according to any one of claims 41 to 46, wherein the variant AAV capsid protein comprises an amino acid sequence with 100% sequence identity to the amino acid sequence set forth in SEQ ID NO:170.

48. The rAAV according to any one of claims 41 to 47, wherein the rAAV comprises a heterologous acid comprising from 5′ to 3′: (a) an inverted terminal repeat (b) a promoter (c) a nucleotide sequence encoding a polypeptide according to any one of claims 32-39 (d) a polyadenylation sequence and/or a WPRE sequence and (e) an inverted terminal repeat.

49. The rAAV according claim 48, wherein the rAAV wherein the inverted terminal repeat is an AAV2 inverted terminal repeat.

50. The rAAV according to any one of claims 41 to 49, wherein the promoter is a ubiquitous promoter.

51. The rAAV according to claim 50, wherein the promoter is a CAG promoter

52. The rAAV according to any one of claims 41 to 49, wherein the promoter is a tissue-specific promoter.

53. A host cell comprising the rAAV according to any one of claims 41 to 52.

54. A pharmaceutical composition comprising the rAAV according to any one of claims 41 to 52 and a pharmaceutically acceptable carrier, diluent, excipient or buffer.

55. A method for treating dry age-related macular disorder (dry AMD) in a subject in need thereof, comprising is administering to the subject a therapeutically effective amount of the rAAV according to any one of claims 41 to 52 or the pharmaceutical composition according to claim 54.

56. The method according to claim 55, wherein the rAAV or the pharmaceutical composition is administered to the subject by periocular, intravitreal, suprachoroidal or subretinal administration at a dosage from about 10⁸ vector genomes (vg)/eye to about 10¹³ vg/eye, or preferably dosage from about 6×10⁹ vg/eye to about 6×10¹⁰ vg/eye.

57. The method according to claim 56, wherein the rAAV or the pharmaceutical composition is administered to the subject by intravitreal administration at a dosage from about 1×10⁹ vg/eye to about 1×10¹⁰ vg/eye.

58. A method for treating geographic atrophy (GA) in a subject in need thereof, comprising administering to the subject a therapeutically effective amount of the rAAV according to any one of claims 41 to 52 or the pharmaceutical composition according to claim 54.

59. The method according to claim 58, wherein the rAAV or the pharmaceutical composition is administered to the subject by periocular, intravitreal, suprachoroidal or subretinal administration at a dosage from about 10⁸ vector genomes (vg)/eye to about 10¹³ vg/eye, or preferably dosage from about 6×10⁹ vg/eye to about 6×10¹⁰ vg/eye.

60. The method according to claim 59, wherein the rAAV or the pharmaceutical composition is administered to the subject by intravitreal administration at a dosage from about 1×10⁹ vg/eye to about 1×10¹⁰ vg/eye.

61. A method for delivering the rAAV according to any one of claims 41 to 52 or the pharmaceutical composition according to claim 54 to an eye of a subject, wherein is wherein the rAAV or the pharmaceutical composition is administered to the subject by periocular, intravitreal, suprachoroidal or subretinal administration.

62. The method according to claim 61, wherein the rAAV or the pharmaceutical composition administered to the eye of the subject by intravitreal administration.

63. An isolated polypeptide comprising a VH or VHH domain that specifically binds to human complement factor C3 (SEQ ID NO:177), wherein the VH or VHH domain binds to an epitope within amino acids 1540-1600 of SEQ ID NO:177 or an amino acid sequence at least 80% identical thereto.

64. The isolated polypeptide according to claim 63, wherein the VH or VHH domain binds to an epitope comprising, consisting essentially of, or consisting of amino acids 1548-1561 of SEQ ID NO:177 or an amino acid sequence at least 80% identical thereto and/or binds to an epitope comprising, consisting essentially of, or consisting of amino acids 1568-1573 of SEQ ID NO:177 or an amino acid sequence at least 80% identical thereto and/or binds to an epitope comprising, consisting essentially of, or consisting of amino acids 1583-1591 of SEQ ID NO:177 or an amino acid sequence at least 80% identical thereto.

65. The isolated polypeptide according to claim 63 or 64, wherein the polypeptide comprises a CDR1 of SEQ ID NO:54 or an amino acid sequence at least 70% identical thereto, a CDR2 of SEQ ID NO:55 or an amino acid sequence at least 70% identical thereto, and a CDR3 of SEQ ID NO:56 or an amino acid sequence at least 70% identical thereto, preferably wherein the polypeptide comprises the amino acid sequence of SEQ ID NO:14 or an amino acid sequence at least 70% identical thereto.

66. An isolated polypeptide comprising a VH or VHH domain that specifically binds to human complement factor C3 (SEQ ID NO:177), wherein the VH or VHH domain binds to a discontinuous epitope comprised within amino acids 110-130 and amino acids 430-510 of SEQ ID NO:177.

67. The isolated polypeptide according to claim 66, wherein the VH or VHH domain binds to an epitope to an epitope comprising, consisting essentially of, or consisting of amino acids 114-124 of SEQ ID NO:177 or an amino acid sequence at least 80% identical thereto and/or binds to an epitope comprising, consisting essentially of, or consisting of amino acids 497-505 of SEQ ID NO:177 or an amino acid sequence at least 80% identical thereto.

68. The isolated polypeptide according to claim 66 or 67, wherein the polypeptide comprises a CDR1 of SEQ ID NO:18 or an amino acid sequence at least 70% identical thereto, a CDR2 of SEQ ID NO:19 or an amino acid sequence at least 70% identical thereto, and a CDR3 of SEQ ID NO:20 or an amino acid sequence at least 70% identical thereto, preferably wherein the polypeptide comprises the amino acid sequence of SEQ ID NO:98 or an amino acid sequence at least 70% identical thereto.

69. An isolated polypeptide comprising a VH or VHH domain that specifically binds to human complement factor C5 (SEQ ID NO:175), wherein the VH or VHH domain competitively inhibits binding of eculizumab to human C5 protein.

70. The isolated polypeptide according to claim 69, wherein the polypeptide comprises a CDR1 of SEQ ID NO:69 or an amino acid sequence at least 70% identical thereto, a CDR2 of SEQ ID NO:70 or an amino acid sequence at least 70% identical thereto, and a CDR3 of SEQ ID NO:71 or an amino acid sequence at least 70% identical thereto, preferably wherein the polypeptide comprises the amino acid sequence of SEQ ID NO:141 or an amino acid sequence at least 70% identical thereto.

71. A heterodimeric fusion protein comprising a first domain comprising a polypeptide according to any one of claims 1-8 and 32-39 and a second domain comprising an immunoglobulin Fc domain, preferably wherein the Fc domain is an IgG4 Fc domain, wherein the first domain and second domain are joined by a linker, optionally wherein the linker is an IgG2 hinge.

72. The fusion protein according to claim 71, wherein the fusion protein comprises the amino acid sequence of any one of SEQ ID Nos: 178-180 or an amino acid sequence at least 70% identical thereto.

73. A homodimeric fusion protein comprising a first domain comprising a polypeptide according to any one of claims 1-8 and 32-39 and a second domain identical to the first domain, preferably wherein the first domain and second domain are joined by a linker, optionally wherein the linker is a poly-Glycine-Serine (G4S) linker, preferably a (G4S)₂ linker.

74. The fusion protein according to claim 73, wherein the fusion protein comprises the amino acid sequence of SEQ ID NO:181 or 182 or an amino acid sequence at least 70% identical thereto.


SEQUENCE LISTING (183 sequences)

SEQ ID NO: 1 - protein, 122 aa, synthetic construct
  REGION 1..122: note = anti-CD3 antibody
QVQLQESGGGLVQAGGSLRLSCAASGNISAPSLMGWYRQAPGKEREFVATITYGSITNYVDSVKGRFTISRDNAKNTVYLQMNSLKPEDTAVYYCAANTLGIYNTYGDHHYWGQGTQVTVSS

SEQ ID NO: 2 - protein, 118 aa, synthetic construct
  REGION 1..118: note = anti-C3 antibody
QVQLQESGGGLVQAGGSLRLSCAASGTIFSRNYMGWYRQAPGKERELVAGIGYGSSTNYADSVKGRFTISRDNAKNTVYLQMNSLKPEDTAVYYCAAVPTYRPYYFYWGQGTQVTVSS

SEQ ID NO: 3 - protein, 122 aa, synthetic construct
  REGION 1..122: note = anti-C3 antibody
QVQLQESGGGLVQAGGSLRLSCAASGTISGPGDMGWYRQAPGKERELVAGINYGAITYYADSVKGRFTISRDNAKNTVYLQMNSLKPEDTAVYYCAADYETWAPSYEYFDYWGQGTQVTVSS

SEQ ID NO: 4 - protein, 122 aa, synthetic construct
  REGION 1..122: note = anti-C3 antibody
QVQLQESGGGLVQAGGSLRLSCAASGTIFDDESMGWYRQAPGKEREFVASINYGSTTYYADSVKGRFTISRDNAKNTVYLQMNSLKPEDTAVYYCAVGWSTNYDWTLSYYYWGQGTLVTVSS

SEQ ID NO: 5 - protein, 118 aa, synthetic construct
  REGION 1..118: note = anti-C3 antibody
QVQLQESGGGLVQAGGSLRLSCAASGNIFVGHGMGWYRQAPGKEREFVAAIGYGAITNYADSVKGRFTISRDNAKNTVYLQMNSLKPEDTAVYYCAASSYYPPRYWYWGQGTLVTVSS

SEQ ID NO: 6 - protein, 122 aa, synthetic construct
  REGION 1..122: note = anti-C3 antibody
QVQLQESGGGLVQAGGSLRLSCAASGYIFTGVDMGWYRQAPGKEREFVAAISYGASTYYADSVKGRFTISRDNAKNTVYLQMNSLKPEDTAVYYCAASLSNTYLLDLFFRYWGQGTQVTVSS

SEQ ID NO: 7 - protein, 126 aa, synthetic construct
  REGION 1..126: note = anti-C3 antibody
QVQLQESGGGLVQAGGSLRLSCAASGTIFPTDRMGWYRQAPGKEREFVATIDLGGTTNYADSVKGRFTISRDNAKNTVYLQMNSLKPEDTAVYYCAAAWEGPSRYGRHRYYSHRYWGQGTQVTVSS

SEQ ID NO: 8 - protein, 118 aa, synthetic construct
  REGION 1..118: note = anti-C3 antibody
QVQLQESGGGLVQAGGSLRLSCAASGYIFVYSTMGWYRQAPGKEREFVASINYGSITYYADSVKGRFTISRDNAKNTVYLQMNSLKPEDTAVYYCAVTDKSRYWLTYWGQGTQVTVSS

SEQ ID NO: 9 - protein, 118 aa, synthetic construct
  REGION 1..118: note = anti-C3 antibody
QVQLQESGGGLVQAGGSLRLSCAASGYISGGSIMGWYRQAPGKEREFVAGINQGSNTYYADSVKGRFTISRDNAKNTVYLQMNSLKPEDTAVYYCAVGGRSYYYHHYWGQGTQVTVSS

SEQ ID NO: 10 - protein, 122 aa, synthetic construct
  REGION 1..122: note = anti-C3 antibody
DVQLVESGGGLVQPGGSLRLSCAASGFTLDNYAIAWFRQAPGKEREGVSCISSSRGSTAYEDSVKGRFTIARDNAKNTVYLQMNSLKPEDTAVYYCAARARGMCREFDYDYWGQGTQVTVSS

SEQ ID NO: 11 - protein, 122 aa, synthetic construct
  REGION 1..122: note = anti-C3 antibody
DVQLVESGGGLVQPGGSLRLSCATSGFTLDYYAIGWFRQAPGKEREGVSCITGPDGSTHYADSVKGRFTISRDNAKNTVYLQMNSLKPEDTAVYYCAATAGLYGCGYGMDHWGKGTQVTVSS

SEQ ID NO: 12 - protein, 120 aa, synthetic construct
  REGION 1..120: note = anti-C3 antibody
DVQLVESGGGLVQPGGSLRLSCATSGFSFGVYDMSWVRQASGKGLEWVSGIDSGSNTAYADSVKVRFTISRDNAKNTLYLQMNNLKPEDTAVYYCALGRETDYIPVTDFRTQGTQVTVSS

SEQ ID NO: 13 - protein, 120 aa, synthetic construct
  REGION 1..120: note = anti-C3 antibody
DVQLVESGGGLVQPGGSLRLSCTASGFTFGVYAMSWVRQAPGKGLEWVSSIDSGVNTAYADSVKGRFTISRDNAINTVFLQMNSLKPEDTAVYHCALGRESDYEPVTGSWGQGTQVTVSS

SEQ ID NO: 14 - protein, 118 aa, synthetic construct
  REGION 1..118: note = anti-C3 antibody
DVQLVESGGGLVQAGGSLRLACAASGLTFSPYAMGWFRQAPGKEREFVAGIRWISGNPYYADSVKGRFTISRDNAKNTVYLQMNSLKPEDTAVYSCAAAPLTRSYDYWGQGTQVTVSS

SEQ ID NO: 15 - protein, 9 aa, synthetic construct
  REGION 1..9: note = anti-C3 CDR1
GNISAPSLM

SEQ ID NO: 16 - protein, 10 aa, synthetic construct
  REGION 1..10: note = anti-C3 CDR2
TITYGSITNY

SEQ ID NO: 17 - protein, 14 aa, synthetic construct
  REGION 1..14: note = anti-C3 CDR3
NTLGIYNTYGDHHY

SEQ ID NO: 18 - protein, 9 aa, synthetic construct
  REGION 1..9: note = anti-C3 CDR1
GTIFSRNYM

SEQ ID NO: 19 - protein, 10 aa, synthetic construct
  REGION 1..10: note = anti-C3 CDR3
GIGYGSSTNY

SEQ ID NO: 20 - protein, 10 aa, synthetic construct
  REGION 1..10: note = anti-C3 CDR3
VPTYRPYYFY

SEQ ID NO: 21 - protein, 10 aa, synthetic construct
  REGION 1..10: note = anti-C3 CDR1
GTISGPGDMG

SEQ ID NO: 22 - protein, 10 aa, synthetic construct
  REGION 1..10: note = anti-C3 CDR2
GINYGAITYY

SEQ ID NO: 23 - protein, 14 aa, synthetic construct
  REGION 1..14: note = anti-C3 CDR3
DYETWAPSYEYFDY

SEQ ID NO: 24 - protein, 10 aa, synthetic construct
  REGION 1..10: note = anti-C3 CDR1
GTIFDDESMG

SEQ ID NO: 25 - protein, 10 aa, synthetic construct
  REGION 1..10: note = anti-C3 CDR2
SINYGSTTYY

SEQ ID NO: 26 - protein, 14 aa, synthetic construct
  REGION 1..14: note = anti-C3 CDR3
GWSTNYDWTLSYYY

SEQ ID NO: 27 - protein, 10 aa, synthetic construct
  REGION 1..10: note = anti-C3 CDR1
GNIFVGHGMG

SEQ ID NO: 28 - protein, 10 aa, synthetic construct
  REGION 1..10: note = anti-C3 CDR2
AIGYGAITNY

SEQ ID NO: 29 - protein, 10 aa, synthetic construct
  REGION 1..10: note = anti-C3 CDR3
SSYYPPRYWY

SEQ ID NO: 30 - protein, 10 aa, synthetic construct
  REGION 1..10: note = anti-C3 CDR1
GYIFTGVDMG

SEQ ID NO: 31 - protein, 10 aa, synthetic construct
  REGION 1..10: note = anti-C3 CDR2
AISYGASTYY

SEQ ID NO: 32 - protein, 14 aa, synthetic construct
  REGION 1..14: note = anti-C3 CDR3
SLSNTYLLDLFFRY

SEQ ID NO: 33 - protein, 10 aa, synthetic construct
  REGION 1..10: note = anti-C3 CDR1
GTIFPTDRMG

SEQ ID NO: 34 - protein, 10 aa, synthetic construct
  REGION 1..10: note = anti-C3 CDR2
TIDLGGTTNY

SEQ ID NO: 35 - protein, 18 aa, synthetic construct
  REGION 1..18: note = anti-C3 CDR3
AWEGPSRYGRHRYYSHRY

SEQ ID NO: 36 - protein, 10 aa, synthetic construct
  REGION 1..10: note = anti-C3 CDR1
GYIFVYSTMG

SEQ ID NO: 37 - protein, 10 aa, synthetic construct
  REGION 1..10: note = anti-C3 CDR2
SINYGSITYY

SEQ ID NO: 38 - protein, 10 aa, synthetic construct
  REGION 1..10: note = anti-C3 CDR3
TDKSRYWLTY

SEQ ID NO: 39 - protein, 10 aa, synthetic construct
  REGION 1..10: note = anti-C3 CDR1
GYISGGSIMG

SEQ ID NO: 40 - protein, 10 aa, synthetic construct
  REGION 1..10: note = anti-C3 CDR2
GINQGSNTYY

SEQ ID NO: 41 - protein, 10 aa, synthetic construct
  REGION 1..10: note = anti-C3 CDR3
GGRSYYYHHY

SEQ ID NO: 42 - protein, 10 aa, synthetic construct
  REGION 1..10: note = anti-C3 CDR1
GFTLDNYAIA

SEQ ID NO: 43 - protein, 11 aa, synthetic construct
  REGION 1..11: note = anti-C3 CDR2
CISSSRGSTAY

SEQ ID NO: 44 - protein, 13 aa, synthetic construct
  REGION 1..13: note = anti-C3 CDR3
RARGMCREFDYDY

SEQ ID NO: 45 - protein, 10 aa, synthetic construct
  REGION 1..10: note = anti-C3 CDR1
GFTLDYYAIG

SEQ ID NO: 46 - protein, 11 aa, synthetic construct
  REGION 1..11: note = anti-C3 CDR2
CITGPDGSTHY

SEQ ID NO: 47 - protein, 13 aa, synthetic construct
  REGION 1..13: note = anti-C3 CDR3
TAGLYGCGYGMDH

SEQ ID NO: 48 - protein, 10 aa, synthetic construct
  REGION 1..10: note = anti-C3 CDR1
GFSFGVYDMS

SEQ ID NO: 49 - protein, 10 aa, synthetic construct
  REGION 1..10: note = anti-C3 CDR2
GIDSGSNTAY

SEQ ID NO: 50 - protein, 12 aa, synthetic construct
  REGION 1..12: note = anti-C3 CDR3
GRETDYIPVTDF

SEQ ID NO: 51 - protein, 10 aa, synthetic construct
  REGION 1..10: note = anti-C3 CDR1
GFTFGVYAMS

SEQ ID NO: 52 - protein, 10 aa, synthetic construct
  REGION 1..10: note = anti-C3 CDR2
SIDSGVNTAY

SEQ ID NO: 53 - protein, 12 aa, synthetic construct
  REGION 1..12: note = anti-C3 CDR3
GRESDYEPVTGS

SEQ ID NO: 54 - protein, 10 aa, synthetic construct
  REGION 1..10: note = anti-C3 CDR1
GLTFSPYAMG

SEQ ID NO: 55 - protein, 11 aa, synthetic construct
  REGION 1..11: note = anti-C3 CDR2
GIRWISGNPYY

SEQ ID NO: 56 - protein, 9 aa, synthetic construct
  REGION 1..9: note = anti-C3 CDR3
APLTRSYDY

SEQ ID NO: 57 - protein, 118 aa, synthetic construct
  REGION 1..118: note = anti-C5 antibody
QVQLQESGGGLVQAGGSLRLSCAASGYISYDDDMGWYRQAPGKEREFVAAIDVGGSTYYADSVKGRFTISRDNAKNTVYLQMNSLKPEDTAVYYCAVRDGYYWDHDYWGQGTQVTVSS

SEQ ID NO: 58 - protein, 118 aa, synthetic construct
  REGION 1..118: note = anti-C5 antibody
QVQLQESGGGLVQAGGSLRLSCAASGNIFHQSDMGWYRQAPGKEREFVAAIDYGTNTYYADSVKGRFTISRDNAKNTVYLQMNSLKPEDTAVYYCAARDTSYWDHVYWGQGTQVTVSS

SEQ ID NO: 59 - protein, 122 aa, synthetic construct
  REGION 1..122: note = anti-C5 antibody
QVQLQESGGGLVQAGGSLRLSCAASGSISFDTDMGWYRQAPGKEREFVATIDGGTSTYYADSVKGRFTISRDNAKNTVYLQMNSLKPEDTAVYYCAAEAGYFSLLGPYFRYWGQGTQVTVSS

SEQ ID NO: 60 - protein, 118 aa, synthetic construct
  REGION 1..118: note = anti-C5 antibody
QVQLQESGGGLVQAGGSLRLSCAASGYIFAATDMGWYRQAPGKEREFVATIDYGANTNYADSVKGRFTIGRDNAKNTVYLQMNSLKPEDTAVYYCAARDNNYYDLVYWGQGTQVTVSS

SEQ ID NO: 61 - protein, 118 aa, synthetic construct
  REGION 1..118: note = anti-C5 antibody
QVQLQESGGGLVQAGGSLRLSCAASGTISPYEGMGWYRQAPGKERELVAAIDGGGITYYADSVKGRFTISRDNAKNTVYLQMNSLKPEDTAVYYCAAGIWTLSYYDYWGQGTQVTVSS

SEQ ID NO: 62 - protein, 122 aa, synthetic construct
  REGION 1..122: note = anti-C5 antibody
QVQLQESGGGLVQAGGSLRLSCAASGTIFYSYKMGWYRQAPGKEREFVAGITYGGSTYYADSVKGRFTISRDNAKNTVYLQMNSLKPEDTAVYYCAARAYYVSVYDTALYYWGQGTQVTVSS

SEQ ID NO: 63 - protein, 118 aa, synthetic construct
  REGION 1..118: note = anti-C5 antibody
QVQLQESGGGLVQAGGSLRLSCAASGYIFYDDDMGWYRQAPGKEREFVATIDVGGNTNYADSVKGRFTIGRDNAKNTVYLQMNSLKPEDTAVYYCAVRDNNYYDHVYWGQGTQVTVSS

SEQ ID NO: 64 - protein, 118 aa, synthetic construct
  REGION 1..118: note = anti-C5 antibody
QVQLQESGGGLVQAGGSLRLSCAASGNIFDEWDMGWYRQAPGKEREFVAAIDDGASTYYADSVKGRFTISRDNAKNTVYLQMNSLKPEDTAVYYCAARQTYAWDYRYWGQGTQVTVSS

SEQ ID NO: 65 - protein, 118 aa, synthetic construct
  REGION 1..118: note = anti-C5 antibody
QVQLQESGGGLVQAGGSLRLSCAASGYIFPSDDMGWYRQAPGKEREFVAAINDGASTYYADSVKGRFTISRDNAKNTVYLQMNSLKPEDTAVYYCAVQLNYYWDYVYWGQGTQVTVSS

SEQ ID NO: 66 - protein, 10 aa, synthetic construct
  REGION 1..10: note = anti-C5 CDR1
GYISYDDDMG

SEQ ID NO: 67 - protein, 9 aa, synthetic construct
  REGION 1..9: note = anti-C5 CDR2
AIDVGGSTY

SEQ ID NO: 68 - protein, 10 aa, synthetic construct
  REGION 1..10: note = anti-C5 CDR3
RDGYYWDHDY

SEQ ID NO: 69 - protein, 10 aa, synthetic construct
  REGION 1..10: note = anti-C5 CDR1
GNIFHQSDMG

SEQ ID NO: 70 - protein, 9 aa, synthetic construct
  REGION 1..9: note = anti-C5 CDR2
AIDYGTNTY

SEQ ID NO: 71 - protein, 10 aa, synthetic construct
  REGION 1..10: note = anti-C5 CDR3
RDTSYWDHVY

SEQ ID NO: 72 - protein, 10 aa, synthetic construct
  REGION 1..10: note = C5S3 CDR2
GSISFDTDMG

SEQ ID NO: 73 - protein, 9 aa, synthetic construct
  REGION 1..9: note = anti-C5 CDR2
TIDGGTSTY

SEQ ID NO: 74 - protein, 14 aa, synthetic construct
  REGION 1..14: note = anti-C5 CDR3
EAGYFSLLGPYFRY

SEQ ID NO: 75 - protein, 10 aa, synthetic construct
  REGION 1..10: note = anti-C5 CDR1
GYIFAATDMG

SEQ ID NO: 76 - protein, 9 aa, synthetic construct
  REGION 1..9: note = anti-C5 CDR2
TIDYGANTN

SEQ ID NO: 77 - protein, 10 aa, synthetic construct
  REGION 1..10: note = anti-C5 CDR3
RDNNYYDLVY

SEQ ID NO: 78 - protein, 10 aa, synthetic construct
  REGION 1..10: note = anti-C5 CDR1
GTISPYEGMG

SEQ ID NO: 79 - protein, 9 aa, synthetic construct
  REGION 1..9: note = anti-C5 CDR2
AIDGGGITY

SEQ ID NO: 80 - protein, 10 aa, synthetic construct
  REGION 1..10: note = anti-C5 CDR3
GIWTLSYYDY

SEQ ID NO: 81 - protein, 10 aa, synthetic construct
  REGION 1..10: note = anti-C5 CDR1
GTIFYSYKMG

SEQ ID NO: 82 - protein, 9 aa, synthetic construct
  REGION 1..9: note = anti-C5 CDR2
GITYGGSTY

SEQ ID NO: 83 - protein, 14 aa, synthetic construct
  REGION 1..14: note = anti-C5 CDR3
RAYYVSVYDTALYY

SEQ ID NO: 84 - protein, 10 aa, synthetic construct
  REGION 1..10: note = anti-C5 CDR1
GYIFYDDDMG

SEQ ID NO: 85 - protein, 9 aa, synthetic construct
  REGION 1..9: note = anti-C5 CDR2
TIDVGGNTN

SEQ ID NO: 86 - protein, 10 aa, synthetic construct
  REGION 1..10: note = anti-C5 CDR3
RDNNYYDHVY

SEQ ID NO: 87 - protein, 10 aa, synthetic construct
  REGION 1..10: note = anti-C5 CDR1
GNIFDEWDMG

SEQ ID NO: 88 - protein, 9 aa, synthetic construct
  REGION 1..9: note = anti-C5 CDR2
AIDDGASTY

SEQ ID NO: 89 - protein, 10 aa, synthetic construct
  REGION 1..10: note = anti-C5 CDR3
RQTYAWDYRY

SEQ ID NO: 90 - protein, 10 aa, synthetic construct
  REGION 1..10: note = anti-C5 CDR1
GYIFPSDDMG

SEQ ID NO: 91 - protein, 9 aa, synthetic construct
  REGION 1..9: note = anti-C5 CDR2
AINDGASTY

SEQ ID NO: 92 - protein, 10 aa, synthetic construct
  REGION 1..10: note = anti-C5 CDR3
QLNYYWDYVY

SEQ ID NO: 93 - protein, 14 aa, synthetic construct
  REGION 1..14: note = Histidine tag
WSHPQFEKHHHHHH

SEQ ID NO: 94 - protein, 122 aa, synthetic construct
  REGION 1..122: note = humanized anti-C3 antibody
EVQLVESGGGLVQPGGSLRLSCAASGNISAPSLMGWYRQAPGKEREFVATITYGSITNYADSVKGRFTISRDNSKNTLYLQMNSLRAEDTAVYYCAANTLGIYNTYGDHHYWGQGTLVTVSS

SEQ ID NO: 95 - protein, 122 aa, synthetic construct
  REGION 1..122: note = humanized anti-C3 antibody
EVQLVESGGGLVQAGGSLRLSCAASGNISAPSLMGWYRQAPGKEREFVATITYGSITNYVDSVKGRFTISRDNSKNTLYLQMNSLRPEDTAVYYCAANTLGIYNTYGDHHYWGQGTLVTVSS

SEQ ID NO: 96 - protein, 122 aa, synthetic construct
  REGION 1..122: note = humanized anti-C3 antibody
QVQLVESGGGLVQAGGSLRLSCAASGNISAPSLMGWYRQAPGKEREFVATITYGSITNYVDSVKGRFTISRDNSKNTLYLQMNSLRPEDTAVYYCAANTLGIYNTYGDHHYWGQGTLVTVSS

SEQ ID NO: 97 - protein, 118 aa, synthetic construct
  REGION 1..118: note = humanized anti-C3 antibody
EVQLVESGGGLVQPGGSLRLSCAASGTIFSRNYMGWYRQAPGKERELVAGIGYGSSTNYADSVKGRFTISRDNSKNTLYLQMNSLRAEDTAVYYCAAVPTYRPYYFYWGQGTLVTVSS

SEQ ID NO: 98 - protein, 118 aa, synthetic construct
  REGION 1..118: note = humanized anti-C3 antibody
EVQLVESGGGLVQAGGSLRLSCAASGTIFSRNYMGWYRQAPGKERELVAGIGYGSSTNYADSVKGRFTISRDNSKNTLYLQMNSLRPEDTAVYYCAAVPTYRPYYFYWGQGTLVTVSS

SEQ ID NO: 99 - protein, 118 aa, synthetic construct
  REGION 1..118: note = humanized anti-C3 antibody
QVQLVESGGGLVQAGGSLRLSCAASGTIFSRNYMGWYRQAPGKERELVAGIGYGSSTNYADSVKGRFTISRDNSKNTLYLQMNSLRPEDTAVYYCAAVPTYRPYYFYWGQGTLVTVSS

SEQ ID NO: 100 - protein, 122 aa, synthetic construct
  REGION 1..122: note = humanized anti-C3 antibody
EVQLVESGGGLVQPGGSLRLSCAASGTISGPGDMGWYRQAPGKERELVAGINYGAITYYADSVKGRFTISRDNSKNTLYLQMNSLRAEDTAVYYCAADYETWAPSYEYFDYWGQGTLVTVSS

SEQ ID NO: 101 - protein, 122 aa, synthetic construct
  REGION 1..122: note = humanized anti-C3 antibody
EVQLVESGGGLVQAGGSLRLSCAASGTISGPGDMGWYRQAPGKERELVAGINYGAITYYADSVKGRFTISRDNSKNTLYLQMNSLRPEDTAVYYCAADYETWAPSYEYFDYWGQGTLVTVSS

SEQ ID NO: 102 - protein, 122 aa, synthetic construct
  REGION 1..122: note = humanized anti-C3 antibody
QVQLVESGGGLVQAGGSLRLSCAASGTISGPGDMGWYRQAPGKERELVAGINYGAITYYADSVKGRFTISRDNSKNTLYLQMNSLRPEDTAVYYCAADYETWAPSYEYFDYWGQGTLVTVSS

SEQ ID NO: 103 - protein, 122 aa, synthetic construct
  REGION 1..122: note = humanized anti-C3 antibody
EVQLVESGGGLVQPGGSLRLSCAASGTIFDDESMGWYRQAPGKEREFVASINYGSTTYYADSVKGRFTISRDNAKNSLYLQMNSLRAEDTAVYYCAVGWSTNYDWTLSYYYWGQGTLVTVSS

SEQ ID NO: 104 - protein, 122 aa, synthetic construct
  REGION 1..122: note = humanized anti-C3 antibody
EVQLVESGGGLVQAGGSLRLSCAASGTIFDDESMGWYRQAPGKEREFVASINYGSTTYYADSVKGRFTISRDNAKNSLYLQMNSLRPEDTAVYYCAVGWSTNYDWTLSYYYWGQGTLVTVSS

SEQ ID NO: 105 - protein, 122 aa, synthetic construct
  REGION 1..122: note = humanized anti-C3 antibody
QVQLVESGGGLVQAGGSLRLSCAASGTIFDDESMGWYRQAPGKEREFVASINYGSTTYYADSVKGRFTISRDNAKNSLYLQMNSLRPEDTAVYYCAVGWSTNYDWTLSYYYWGQGTLVTVSS

SEQ ID NO: 106 - protein, 118 aa, synthetic construct
  REGION 1..118: note = humanized anti-C3 antibody
EVQLVESGGGLVQPGGSLRLSCAASGNIFVGHGMGWYRQAPGKEREFVAAIGYGAITNYADSVKGRFTISRDNSKNTLYLQMNSLRAEDTAVYYCAASSYYPPRYWYWGQGTLVTVSS

SEQ ID NO: 107 - protein, 118 aa, synthetic construct
  REGION 1..118: note = humanized anti-C3 antibody
EVQLVESGGGLVQAGGSLRLSCAASGNIFVGHGMGWYRQAPGKEREFVAAIGYGAITNYADSVKGRFTISRDNSKNTLYLQMNSLRPEDTAVYYCAASSYYPPRYWYWGQGTLVTVSS

SEQ ID NO: 108 - protein, 118 aa, synthetic construct
  REGION 1..118: note = humanized anti-C3 antibody
QVQLVESGGGLVQAGGSLRLSCAASGNIFVGHGMGWYRQAPGKEREFVAAIGYGAITNYADSVKGRFTISRDNSKNTLYLQMNSLRPEDTAVYYCAASSYYPPRYWYWGQGTLVTVSS

SEQ ID NO: 109 - protein, 122 aa, synthetic construct
  REGION 1..122: note = humanized anti-C3 antibody
EVQLVESGGGLVQPGGSLRLSCAASGYIFTGVDMGWYRQAPGKEREFVAAISYGASTYYADSVKGRFTISRDNSKNTLYLQMNSLRAEDTAVYYCAASLSNTYLLDLFFRYWGQGTLVTVSS

SEQ ID NO: 110 - protein, 122 aa, synthetic construct
  REGION 1..122: note = humanized anti-C3 antibody
EVQLVESGGGLVQAGGSLRLSCAASGYIFTGVDMGWYRQAPGKEREFVAAISYGASTYYADSVKGRFTISRDNSKNTLYLQMNSLRPEDTAVYYCAASLSNTYLLDLFFRYWGQGTLVTVSS

SEQ ID NO: 111 - protein, 122 aa, synthetic construct
  REGION 1..122: note = humanized anti-C3 antibody
QVQLVESGGGLVQAGGSLRLSCAASGYIFTGVDMGWYRQAPGKEREFVAAISYGASTYYADSVKGRFTISRDNSKNTLYLQMNSLRPEDTAVYYCAASLSNTYLLDLFFRYWGQGTLVTVSS

SEQ ID NO: 112 - protein, 126 aa, synthetic construct
  REGION 1..126: note = humanized anti-C3 antibody
EVQLVESGGGLVQPGGSLRLSCAASGTIFPTDRMGWYRQAPGKEREFVATIDLGGTTNYADSVKGRFTISRDNSKNTLYLQMNSLRAEDTAVYYCAAAWEGPSRYGRHRYYSHRYWGQGTLVTVSS

SEQ ID NO: 113 - protein, 126 aa, synthetic construct
  REGION 1..126: note = humanized anti-C3 antibody
EVQLVESGGGLVQAGGSLRLSCAASGTIFPTDRMGWYRQAPGKEREFVATIDLGGTTNYADSVKGRFTISRDNSKNTLYLQMNSLRAEDTAVYYCAAAWEGPSRYGRHRYYSHRYWGQGTLVTVSS

SEQ ID NO: 114 - protein, 126 aa, synthetic construct
  REGION 1..126: note = humanized anti-C3 antibody
QVQLVESGGGLVQAGGSLRLSCAASGTIFPTDRMGWYRQAPGKEREFVATIDLGGTTNYADSVKGRFTISRDNSKNTLYLQMNSLRPEDTAVYYCAAAWEGPSRYGRHRYYSHRYWGQGTLVTVSS

SEQ ID NO: 115 - protein, 118 aa, synthetic construct
  REGION 1..118: note = humanized anti-C3 antibody
EVQLVESGGGLVQPGGSLRLSCAASGYIFVYSTMGWYRQAPGKEREFVASINYGSITYYADSVKGRFTISRDNSKNTLYLQMNSLRAEDTAVYYCAVTDKSRYWLTYWGQGTLVTVSS

SEQ ID NO: 116 - protein, 118 aa, synthetic construct
  REGION 1..118: note = humanized anti-C3 antibody
EVQLVESGGGLVQAGGSLRLSCAASGYIFVYSTMGWYRQAPGKEREFVASINYGSITYYADSVKGRFTISRDNSKNTLYLQMNSLRPEDTAVYYCAVTDKSRYWLTYWGQGTLVTVSS

SEQ ID NO: 117 - protein, 118 aa, synthetic construct
  REGION 1..118: note = humanized anti-C3 antibody
QVQLVESGGGLVQAGGSLRLSCAASGYIFVYSTMGWYRQAPGKEREFVASINYGSITYYADSVKGRFTISRDNSKNTLYLQMNSLRPEDTAVYYCAVTDKSRYWLTYWGQGTLVTVSS

SEQ ID NO: 118 - protein, 118 aa, synthetic construct
  REGION 1..118: note = humanized anti-C3 antibody
EVQLVESGGGLVQPGGSLRLSCAASGYISGGSIMGWYRQAPGKEREFVAGINQGSNTYYADSVKGRFTISRDNSKNTLYLQMNSLRAEDTAVYYCAVGGRSYYYHHYWGQGTLVTVSS

SEQ ID NO: 119 - protein, 118 aa, synthetic construct
  REGION 1..118: note = humanized anti-C3 antibody
EVQLVESGGGLVQAGGSLRLSCAASGYISGGSIMGWYRQAPGKEREFVAGINQGSNTYYADSVKGRFTISRDNSKNTLYLQMNSLRPEDTAVYYCAVGGRSYYYHHYWGQGTLVTVSS

SEQ ID NO: 120 - protein, 118 aa, synthetic construct
  REGION 1..118: note = humanized anti-C3 antibody
QVQLVESGGGLVQAGGSLRLSCAASGYISGGSIMGWYRQAPGKEREFVAGINQGSNTYYADSVKGRFTISRDNSKNTLYLQMNSLRPEDTAVYYCAVGGRSYYYHHYWGQGTLVTVSS

SEQ ID NO: 121 - protein, 122 aa, synthetic construct
  REGION 1..122: note = humanized anti-C3 antibody
EVQLLESGGGLVQPGGSLRLSCAASGFTLDNYAIAWFRQAPGKEREGVSCISSSRGSTAYEDSVKGRFTISRDNSKNTLYLQMNSLRAEDTAVYYCAARARGMCREFDYDYWGQGTLVTVSS

SEQ ID NO: 122 - protein, 122 aa, synthetic construct
  REGION 1..122: note = humanized anti-C3 antibody
EVQLLESGGGLVQPGGSLRLSCAASGFTLDNYAIAWFRQAPGKEREGVSCISSSRGSTAYEDSVKGRFTISRDNSKNTLYLQMNSLRPEDTAVYYCAARARGMCREFDYDYWGQGTLVTVSS

SEQ ID NO: 123 - protein, 122 aa, synthetic construct
  REGION 1..122: note = humanized anti-C3 antibody
EVQLLESGGGLVQPGGSLRLSCAASGFTLDNYAIAWFRQAPGKEREGVSCISSSRGSTAYEDSVKGRFTISRDNSKNTLYLQMNSLKPEDTAVYYCAARARGMCREFDYDYWGQGTLVTVSS

SEQ ID NO: 124 - protein, 122 aa, synthetic construct
  REGION 1..122: note = humanized anti-C3 antibody
EVQLLESGGGLVQPGGSLRLSCATSGFTLDYYAIGWFRQAPGKEREGVSCITGPDGSTHYADSVKGRFTISRDNSKNTLYLQMNSLRAEDTAVYYCAATAGLYGCGYGMDHWGKGTLVTVSS

SEQ ID NO: 125 - protein, 122 aa, synthetic construct
  REGION 1..122: note = humanized anti-C3 antibody
EVQLLESGGGLVQPGGSLRLSCATSGFTLDYYAIGWFRQAPGKEREGVSCITGPDGSTHYADSVKGRFTISRDNSKNTLYLQMNSLRPEDTAVYYCAATAGLYGCGYGMDHWGKGTLVTVSS

SEQ ID NO: 126 - protein, 122 aa, synthetic construct
  REGION 1..122: note = humanized anti-C3 antibody
EVQLLESGGGLVQPGGSLRLSCATSGFTLDYYAIGWFRQAPGKEREGVSCITGPDGSTHYADSVKGRFTISRDNSKNTLYLQMNSLKPEDTAVYYCAATAGLYGCGYGMDHWGKGTLVTVSS

SEQ ID NO: 127 - protein, 120 aa, synthetic construct
  REGION 1..120: note = humanized anti-C3 antibody
EVQLLESGGGLVQPGGSLRLSCATSGFSFGVYDMSWVRQASGKGLEWVSGIDSGSNTAYADSVKGRFTISRDNSKNTLYLQMNSLRAEDTAVYYCALGRETDYIPVTDFRTQGTLVTVSS

SEQ ID NO: 128 - protein, 120 aa, synthetic construct
  REGION 1..120: note = humanized anti-C3 antibody
EVQLLESGGGLVQPGGSLRLSCATSGFSFGVYDMSWVRQASGKGLEWVSGIDSGSNTAYADSVKGRFTISRDNSKNTLYLQMNNLRPEDTAVYYCALGRETDYIPVTDFRTQGTLVTVSS

SEQ ID NO: 129 - protein, 120 aa, synthetic construct
  REGION 1..120: note = humanized anti-C3 antibody
EVQLLESGGGLVQPGGSLRLSCATSGFSFGVYDMSWVRQASGKGLEWVSGIDSGSNTAYADSVKVRFTISRDNSKNTLYLQMNNLKPEDTAVYYCALGRETDYIPVTDFRTQGTLVTVSS

SEQ ID NO: 130 - protein, 120 aa, synthetic construct
  REGION 1..120: note = humanized anti-C3 antibody
EVQLLESGGGLVQPGGSLRLSCTASGFTFGVYAMSWVRQAPGKGLEWVSSIDSGVNTAYADSVKGRFTISRDNSKNTLYLQMNSLRAEDTAVYHCALGRESDYEPVTGSWGQGTLVTVSS

SEQ ID NO: 131 - protein, 120 aa, synthetic construct
  REGION 1..120: note = humanized anti-C3 antibody
EVQLLESGGGLVQPGGSLRLSCTASGFTFGVYAMSWVRQAPGKGLEWVSSIDSGVNTAYADSVKGRFTISRDNSINTLYLQMNSLRPEDTAVYHCALGRESDYEPVTGSWGQGTLVTVSS

SEQ ID NO: 132 - protein, 120 aa, synthetic construct
  REGION 1..120: note = humanized anti-C3 antibody
EVQLLESGGGLVQPGGSLRLSCTASGFTFGVYAMSWVRQAPGKGLEWVSSIDSGVNTAYADSVKGRFTISRDNSINTLFLQMNSLKPEDTAVYHCALGRESDYEPVTGSWGQGTLVTVSS

SEQ ID NO: 133 - protein, 118 aa, synthetic construct
  REGION 1..118: note = humanized anti-C3 antibody
QVQLVESGGGLVQPGGSLRLACAASGLTFSPYAMGWFRQAPGKEREFVAGIRWISGNPYYADSVKGRFTISRDNSKNTLYLQMNSLRAEDTAVYSCAAAPLTRSYDYWGQGTQVTVSS

SEQ ID NO: 134 - protein, 118 aa, synthetic construct
  REGION 1..118: note = humanized anti-C3 antibody
EVQLVESGGGLVQPGGSLRLACAASGLTFSPYAMGWFRQAPGKEREFVAGIRWISGNPYYADSVKGRFTISRDNSKNTLYLQMNSLRAEDTAVYSCAAAPLTRSYDYWGQGTLVTVSS

SEQ ID NO: 135 - protein, 118 aa, synthetic construct
  REGION 1..118: note = humanized anti-C3 antibody
EVQLVESGGGLVQAGGSLRLACAASGLTFSPYAMGWFRQAPGKEREFVAGIRWISGNPYYADSVKGRFTISRDNSKNTLYLQMNSLRPEDTAVYSCAAAPLTRSYDYWGQGTLVTVSS

SEQ ID NO: 136 - protein, 118 aa, synthetic construct
  REGION 1..118: note = humanized anti-C3 antibody
EVQLVESGGGLVQAGGSLRLACAASGLTFSPYAMGWFRQAPGKEREFVAGIRWISGNPYYADSVKGRFTISRDNSKNTLYLQMNSLKPEDTAVYSCAAAPLTRSYDYWGQGTLVTVSS

SEQ ID NO: 137 - protein, 118 aa, synthetic construct
  REGION 1..118: note = humanized anti-C5 antibody
EVQLVESGGGLVQPGGSLRLSCAASGYISYDDDMGWYRQAPGKEREFVAAIDVGGSTYYADSVKGRFTISRDNSKNTLYLQMNSLRAEDTAVYYCAVRDGYYWDHDYWGQGTLVTVSS

SEQ ID NO: 138 - protein, 118 aa, synthetic construct
  REGION 1..118: note = humanized anti-C5 antibody
EVQLVESGGGLVQAGGSLRLSCAASGYISYDDDMGWYRQAPGKEREFVAAIDVGGSTYYADSVKGRFTISRDNSKNTLYLQMNSLRPEDTAVYYCAVRDGYYWDHDYWGQGTLVTVSS

SEQ ID NO: 139 - protein, 118 aa, synthetic construct
  REGION 1..118: note = humanized anti-C5 antibody
QVQLVESGGGLVQAGGSLRLSCAASGYISYDDDMGWYRQAPGKEREFVAAIDVGGSTYYADSVKGRFTISRDNSKNTLYLQMNSLKPEDTAVYYCAVRDGYYWDHDYWGQGTLVTVSS

SEQ ID NO: 140 - protein, 118 aa, synthetic construct
  REGION 1..118: note = humanized anti-C5 antibody
EVQLVESGGGLVQPGGSLRLSCAASGNIFHQSDMGWYRQAPGKEREFVAAIDYGTNTYYADSVKGRFTISRDNSKNTLYLQMNSLRAEDTAVYYCAARDTSYWDHVYWGQGTLVTVSS

SEQ ID NO: 141 - protein, 118 aa, synthetic construct
  REGION 1..118: note = humanized anti-C5 antibody
EVQLVESGGGLVQAGGSLRLSCAASGNIFHQSDMGWYRQAPGKEREFVAAIDYGTNTYYADSVKGRFTISRDNSKNTLYLQMNSLRPEDTAVYYCAARDTSYWDHVYWGQGTLVTVSS

SEQ ID NO: 142 - protein, 118 aa, synthetic construct
  REGION 1..118: note = humanized anti-C5 antibody
QVQLVESGGGLVQAGGSLRLSCAASGNIFHQSDMGWYRQAPGKEREFVAAIDYGTNTYYADSVKGRFTISRDNSKNTLYLQMNSLKPEDTAVYYCAARDTSYWDHVYWGQGTLVTVSS

SEQ ID NO: 143 - protein, 122 aa, synthetic construct
  REGION 1..122: note = humanized anti-C5 antibody
EVQLVESGGGLVQPGGSLRLSCAASGSISFDTDMGWYRQAPGKEREFVATIDGGTSTYYADSVKGRFTISRDNSKNTLYLQMNSLRAEDTAVYYCAAEAGYFSLLGPYFRYWGQGTLVTVSS

SEQ ID NO: 144 - protein, 122 aa, synthetic construct
  REGION 1..122: note = humanized anti-C5 antibody
EVQLVESGGGLVQAGGSLRLSCAASGSISFDTDMGWYRQAPGKEREFVATIDGGTSTYYADSVKGRFTISRDNSKNTLYLQMNSLRPEDTAVYYCAAEAGYFSLLGPYFRYWGQGTLVTVSS

SEQ ID NO: 145 - protein, 122 aa, synthetic construct
  REGION 1..122: note = humanized anti-C5 antibody
QVQLVESGGGLVQAGGSLRLSCAASGSISFDTDMGWYRQAPGKEREFVATIDGGTSTYYADSVKGRFTISRDNSKNTLYLQMNSLKPEDTAVYYCAAEAGYFSLLGPYFRYWGQGTLVTVSS

SEQ ID NO: 146 - protein, 118 aa, synthetic construct
  REGION 1..118: note = humanized anti-C5 antibody
EVQLVESGGGLVQPGGSLRLSCAASGYIFAATDMGWYRQAPGKEREFVATIDYGANTNYADSVKGRFTIGRDNSKNTLYLQMNSLRAEDTAVYYCAARDNNYYDLVYWGQGTLVTVSS

SEQ ID NO: 147 - protein, 118 aa, synthetic construct
  REGION 1..118: note = humanized anti-C5 antibody
EVQLVESGGGLVQAGGSLRLSCAASGYIFAATDMGWYRQAPGKEREFVATIDYGANTNYADSVKGRFTIGRDNSKNTLYLQMNSLRPEDTAVYYCAARDNNYYDLVYWGQGTLVTVSS

SEQ ID NO: 148 - protein, 118 aa, synthetic construct
  REGION 1..118: note = humanized anti-C5 antibody
QVQLVESGGGLVQAGGSLRLSCAASGYIFAATDMGWYRQAPGKEREFVATIDYGANTNYADSVKGRFTIGRDNSKNTLYLQMNSLKPEDTAVYYCAARDNNYYDLVYWGQGTLVTVSS

SEQ ID NO: 149 - protein, 118 aa, synthetic construct
  REGION 1..118: note = humanized anti-C5 antibody
EVQLVESGGGLVQPGGSLRLSCAASGTISPYEGMGWYRQAPGKERELVAAIDGGGITYYADSVKGRFTISRDNSKNTLYLQMNSLRAEDTAVYYCAAGIWTLSYYDYWGQGTLVTVSS

SEQ ID NO: 150 - protein, 118 aa, synthetic construct
  REGION 1..118: note = humanized anti-C5 antibody
EVQLVESGGGLVQAGGSLRLSCAASGTISPYEGMGWYRQAPGKERELVAAIDGGGITYYADSVKGRFTISRDNSKNTLYLQMNSLRPEDTAVYYCAAGIWTLSYYDYWGQGTLVTVSS

SEQ ID NO: 151 - protein, 118 aa, synthetic construct
  REGION 1..118: note = humanized anti-C5 antibody
QVQLVESGGGLVQAGGSLRLSCAASGTISPYEGMGWYRQAPGKERELVAAIDGGGITYYADSVKGRFTISRDNSKNTLYLQMNSLKPEDTAVYYCAAGIWTLSYYDYWGQGTLVTVSS

SEQ ID NO: 152 - protein, 122 aa, synthetic construct
  REGION 1..122: note = humanized anti-C5 antibody
EVQLVESGGGLVQPGGSLRLSCAASGTIFYSYKMGWYRQAPGKEREFVAGITYGGSTYYADSVKGRFTISRDNSKNTLYLQMNSLRAEDTAVYYCAARAYYVSVYDTALYYWGQGTLVTVSS

SEQ ID NO: 153 - protein, 122 aa, synthetic construct
  REGION 1..122: note = humanized anti-C5 antibody
EVQLVESGGGLVQAGGSLRLSCAASGTIFYSYKMGWYRQAPGKEREFVAGITYGGSTYYADSVKGRFTISRDNSKNTLYLQMNSLRPEDTAVYYCAARAYYVSVYDTALYYWGQGTLVTVSS

SEQ ID NO: 154 - protein, 122 aa, synthetic construct
  REGION 1..122: note = humanized anti-C5 antibody
QVQLVESGGGLVQAGGSLRLSCAASGTIFYSYKMGWYRQAPGKEREFVAGITYGGSTYYADSVKGRFTISRDNSKNTLYLQMNSLKPEDTAVYYCAARAYYVSVYDTALYYWGQGTLVTVSS

SEQ ID NO: 155 - protein, 118 aa, synthetic construct
  REGION 1..118: note = humanized anti-C5 antibody
EVQLVESGGGLVQPGGSLRLSCAASGYIFYDDDMGWYRQAPGKEREFVATIDVGGNTNYADSVKGRFTIGRDNSKNTLYLQMNSLRAEDTAVYYCAVRDNNYYDHVYWGQGTLVTVSS

SEQ ID NO: 156 - protein, 118 aa, synthetic construct
  REGION 1..118: note = humanized anti-C5 antibody
EVQLVESGGGLVQAGGSLRLSCAASGYIFYDDDMGWYRQAPGKEREFVATIDVGGNTNYADSVKGRFTIGRDNSKNTLYLQMNSLRPEDTAVYYCAVRDNNYYDHVYWGQGTLVTVSS

SEQ ID NO: 157 - protein, 118 aa, synthetic construct
  REGION 1..118: note = humanized anti-C5 antibody
QVQLVESGGGLVQAGGSLRLSCAASGYIFYDDDMGWYRQAPGKEREFVATIDVGGNTNYADSVKGRFTIGRDNSKNTLYLQMNSLKPEDTAVYYCAVRDNNYYDHVYWGQGTLVTVSS

SEQ ID NO: 158 - protein, 118 aa, synthetic construct
  REGION 1..118: note = humanized anti-C5 antibody
EVQLVESGGGLVQPGGSLRLSCAASGNIFDEWDMGWYRQAPGKEREFVAAIDDGASTYYADSVKGRFTISRDNSKNTLYLQMNSLRAEDTAVYYCAARQTYAWDYRYWGQGTLVTVSS

SEQ ID NO: 159 - protein, 118 aa, synthetic construct
  REGION 1..118: note = humanized anti-C5 antibody
EVQLVESGGGLVQAGGSLRLSCAASGNIFDEWDMGWYRQAPGKEREFVAAIDDGASTYYADSVKGRFTISRDNSKNTLYLQMNSLRPEDTAVYYCAARQTYAWDYRYWGQGTLVTVSS

SEQ ID NO: 160 - protein, 118 aa, synthetic construct
  REGION 1..118: note = humanized anti-C5 antibody
QVQLVESGGGLVQAGGSLRLSCAASGNIFDEWDMGWYRQAPGKEREFVAAIDDGASTYYADSVKGRFTISRDNSKNTLYLQMNSLKPEDTAVYYCAARQTYAWDYRYWGQGTLVTVSS

SEQ ID NO: 161 - protein, 118 aa, synthetic construct
  REGION 1..118: note = humanized anti-C5 antibody
EVQLVESGGGLVQPGGSLRLSCAASGYIFPSDDMGWYRQAPGKEREFVAAINDGASTYYADSVKGRFTISRDNSKNTLYLQMNSLRAEDTAVYYCAVQLNYYWDYVYWGQGTLVTVSS

SEQ ID NO: 162 - protein, 118 aa, synthetic construct
  REGION 1..118: note = humanized anti-C5 antibody
EVQLVESGGGLVQAGGSLRLSCAASGYIFPSDDMGWYRQAPGKEREFVAAINDGASTYYADSVKGRFTISRDNSKNTLYLQMNSLRPEDTAVYYCAVQLNYYWDYVYWGQGTLVTVSS

SEQ ID NO: 163 - protein, 118 aa, synthetic construct
  REGION 1..118: note = humanized anti-C5 antibody
QVQLVESGGGLVQAGGSLRLSCAASGYIFPSDDMGWYRQAPGKEREFVAAINDGASTYYADSVKGRFTISRDNSKNTLYLQMNSLKPEDTAVYYCAVQLNYYWDYVYWGQGTLVTVSS

SEQ ID NO: 164 - DNA, 145 bp, synthetic construct
  misc_feature 1..145: note = 5' ITR
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 165 - DNA, 145 bp, synthetic construct
  misc_feature 1..145: note = 3' ITR
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 166 - DNA, 1664 bp, synthetic construct
  misc_feature 1..1664: note = CAG promoter
  source 1..1664: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 167 - DNA, 245 bp, synthetic construct
  misc_feature 1..245: note = SV40 polyadenylation
  source 1..245: mol_type = other DNA
ggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatca

SEQ ID NO: 168 - protein, 7 aa, synthetic construct
  REGION 1..7: note = insertion peptide
ISDQTKH

SEQ ID NO: 169 - protein, 10 aa, synthetic construct
  REGION 1..10: note = insertion peptide
LAISDQTKHA

SEQ ID NO: 170 - protein, 745 aa, synthetic construct
  REGION 1..745: note = Variant AAV Capsid
MAADGYLPDWLEDTLSEGIRQWWKLKPGPPPPKAAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAISDQTKHARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 171 - protein, 61 aa, synthetic construct
  REGION 1..61: note = human C3 epitope
GVDYVYKTRLVKVQLSNDFDEYIMAIEQTIKSGSDEVQVGQQRTFISPIKCREALKLEEKK

SEQ ID NO: 172 - protein, 21 aa, synthetic construct
  REGION 1..21: note = human C3 epitope
ATFGTQVVEKVVLVSLQSGYL

SEQ ID NO: 173 - protein, 21 aa, synthetic construct
  REGION 1..21: note = human C3 epitope
YTYLIMNKGRLLKAGRQVREP

SEQ ID NO: 174 - protein, 1676 aa, Macaca fascicularis
MGLLGILCFLIFLGKTWGQEQTYVISAPKIFRVGASENIVIQVYGYTEAFDATISIKSYPDKKFSYSSGHVHLSSENKFQNSAVLTIQPKQLPGGQNQVSYVYLEVVSKHFSKSKKIPITYDNGFLFIHTDKPVYTPDQSVKVRVYSLNDDLKPAKRETVLTFIDPEGSEIDMVEEIDHIGIISFPDFKIPSNPRYGMWMIQAKYKEDFSTTGTAFFEVKEYVLPHFSVSVEPESNFIGYKNFKNFEITIKARYFYNKVVTEADVYITFGIREDLKDDQKEMMQTAMQNTMLINGIAEVTFDSETAVKELSYYSLEDLNNKYLYIAVTVIESTGGFSEEAEIPGIKYVLSPYKLNLVATPLFLKPGIPYSIKVQVKDALDQLVGGVPVTLNAQTIDVNQETSDLEPRKSVTRVDDGVASFVVNLPSGVTVLEFNVKTDAPDLPDENQAREGYRAIAYSSLSQSYLYIDWTDNHKALLVGEYLNIIVTPKSPYIDKITHYNYLILSKGKIIHFGTREKLSDASYQSINIPVTQNMVPSSRLLVYYIVTGEQTAELVSDSVWLNIEEKCGNQLQVHLSPDADTYSPGQTVSLNMVTGMDSWVALTAVDSAVYGVQRRAKKPLERVFQFLEKSDLGCGAGGGLNNANVFHLAGLTFLTNANADDSQENDEPCKEIIRPRRMLQEKIEEIAAKYKHLVVKKCCYDGVRINHDETCEQRAARISVGPRCVKAFTECCVVASQFRANNSHKDLQLGRLHMKTLLPVSKPEIRSYFPESWLWEVHLVPRRKQLQFALPDSVTTWEIQGVGISNSGICVADTIKAKVFKDVFLEMNIPYSVVRGEQVQLKGTVYNYRTSGMQFCVKMSAVEGICTSESPVIDHQGTKSSKCVRQKVEGSSNHLVTFTVLPLEIGLQNINFSLETSFGKEILVKSLRVVPEGVKRESYSGITLDPRGIYGTISRRKEFPYRIPLDLVPKTEIKRILSVKGLLVGEILSAVLSREGINILTHLPKGSAEAELMSVVPVFYVFHYLETGNHWNIFHSDPLIEKRNLEKKLKEGMVSIMSYRNADYSYSVWKGGSASTWLTAFALRVLGQVHKYVEQNQNSICNSLLWLVENYQLDNGSFKENSQYQPIKLQGTLPVEARENSLYLTAFTVIGIRKAFDICPLVKINTALIKADTFLLENTLPAQSTFTLAISAYALSLGDKTHPQFRSIVSALKREALVKGNPPIYRFWKDSLQHKDSSVPNTGTARMVETTAYALLTSLNLKDINYVNPIIKWLSEEQRYGGGFYSTQDTINAIEGLTEYSLLVKQLRLNMDIDVAYKHKGPLHNYKMTDKNFLGRPVEVLLNDDLVVSTGFGSGLATVHVTTVVHKTSTSEEVCSFYLKIDTQDIEASHYRGYGNSDYKRIVACASYKPSKEESSSGSSHAVMDISLPTGINANEEDLKALVEGVDQLFTDYQIKDGHVILQLNSIPSSDFLCVRFRIFELFEVGFLSPATFTVYEYHRPDKQCTMFYSTSNIKIQKVCEGATCKCIEADCGQMQKELDLTISAETRKQTACNPEIAYAYKVIITSITTENVFVKYKATLLDIYKTGEAVAEKDSEITFIKKVTCTNAELVKGRQYLIMGKEALQIKYNFTFRYIYPLDSLTWIEYWPRDTTCSSCQAFLANLDEFAEDIFLNGC

SEQ ID NO: 175 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 176 - protein, 1663 aa, Macaca fascicularis
MGLTSGPSLLLLLLIHLPLALGTPMYSMITPNVLRLESEETVVLEAHDANGDVPVTVTVHDFPGKKLVLSSEKTVLTPATSHMGSVTIRIPANKEFKSEKGHNKFVTVQATFGAQVVEKVVLVSLQSGYLFIQTDKTIYTPGSTVLCRIFTVNHKLLPVGRTVVVNIENPDGIPVKQDSLSSQNQFGILPLSWDIPELVNMGQWKIRAYYENSPQQVFSTEFEVKEYVLPSFEVIVEPTEKFYYIYNQKGLEVTITARFLYGKKVEGTAFVIFGIQDGEQRISLPESLKRIQIEDGSGDAVLSRKVLLDGVQNPRPEDLVGKSLYVSVTVILHSGSDMVQAERSGIPIVTSPYQIHFTKTPKYFKPGMPFDLMVFVTNPDGSPAYRVPVAVQGEDAVQSLTQGDGVAKLSINTHPSQKPLSITVRTKKRELSEAEQATRTMEAQPYSTVGNSNNYLHLSVPRAELRPGETLNVNFLLRMDRTQEAKIRYYTYLIMNKGKLLKVGRQVREPGQDLVVLPLSITTDFIPSFRLVAYYTLIGANGQREVVADSVWVDVKDSCVGSLVVKSGQSEDRQPLPGQQMTLKIEGDHGARVGLVAVDKGVFVLNKKNKLTQSKIWDVVEKADIGCTPGSGKDYAGVFSDAGLTFASSSGQQTAQRAELQCPQPAARRRRSVQLAEKRMDKVGQYPKELRKCCEHGMRENPMRFSCQRRTRYITLDEACKKAFLDCCNYITELRRQHARASHLGLARSNLDEDIIAEENIVSRSEFPESWLWKIEELKEAPKNGISTKLMNIFLKDSITTWEILAVSLSDKKGICVADPFEVTVMQDFFIDLRLPYSVVRNEQVEIRAVLYNYRQNQELKVRVELLHNPAFCSLATAKRRHQQTVTIPPKSSLSVPYVIVPLKTGQQEVEVKAAVYHFFISDGVRKSLKVVPEGIRMNKTVAVRTLDPERLGQEGVQREDVPPADLSDQVPDTESETRILLQGTPVAQMTEDAIDAERLKHLIVTPSGCGEQNMITMTPTVIAVHYLDETEQWEKFGPEKRQGALELIKKGYTQQLAFRQPSSAFAAFLNRAPSTWLTAYVVKVFSLAVNLIAIDSQVLCGAVKWLILEKQKPDGVFQEDAPVIHQEMTGGFRNTNEKDMALTAFVLISLQEAKEICEEQVNSLPGSITKAGDFLEANYMNLQRSYTVAIAAYALAQMGRLKGPLLNKFLTTAKDKNRWEEPGQQLYNVEATSYALLALLQLKDFDFVPPVVRWLNEQRYYGGGYGSTQATFMVFQALAQYQKDVPDHKELNLDVSLQLPSRSSKIIHRIHWESASLLRSEETKENEGFTVTAEGKGQGTLSVVTMYHAKAKGQLTCNKFDLKVTIKPAPETEKRPQDAKNTMILEICTRYRGDQDATMSILDISMMTGFVPDTDDLKQLANGVDRYISKYELDKAFSDRNTLIIYLDKVSHSEDDCIAFKVHQYFNVELIQPGAVKVYAYYNLAESCTRFYHPEKEDGKLNKLCRDELCRCAEENCFIQKLDDKVTLEERLDKACEPGVDYVYKTRLVKAQLSNDFDEYIMAIEQIIKSGSDEVQVGQQRTFISPIKCREALKLEERKHYLMWGLSSDFWGEKPNLSYIIGKDTWVEHWPEEDECQDEENQKQCQDLGTFTENMVVFGCPN

SEQ ID NO: 177 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 178 - protein, 346 aa, synthetic construct
  REGION 1..346: note = heterodimeric fusion protein
EVQLVESGGGLVQAGGSLRLACAASGLTFSPYAMGWFRQAPGKEREFVAGIRWISGNPYYADSVKGRFTISRDNSKNTLYLQMNSLRPEDTAVYSCAAAPLTRSYDYWGQGTLVTVSSERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 179 - protein, 346 aa, synthetic construct
  REGION 1..346: note = heterodimeric fusion protein
EVQLVESGGGLVQAGGSLRLSCAASGTIFSRNYMGWYRQAPGKERELVAGIGYGSSTNYADSVKGRFTISRDNSKNTLYLQMNSLRPEDTAVYYCAAVPTYRPYYFYWGQGTLVTVSSERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 180 - protein, 346 aa, synthetic construct
  REGION 1..346: note = heterodimeric fusion protein
EVQLVESGGGLVQAGGSLRLSCAASGNIFHQSDMGWYRQAPGKEREFVAAIDYGTNTYYADSVKGRFTISRDNSKNTLYLQMNSLRPEDTAVYYCAARDTSYWDHVYWGQGTLVTVSSERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 181 - protein, 246 aa, synthetic construct
  REGION 1..246: note = homodimeric fusion protein
DVQLVESGGGLVQAGGSLRLACAASGLTFSPYAMGWFRQAPGKEREFVAGIRWISGNPYYADSVKGRFTISRDNAKNTVYLQMNSLKPEDTAVYSCAAAPLTRSYDYWGQGTQVTVSSGGGGSGGGGSDVQLVESGGGLVQAGGSLRLACAASGLTFSPYAMGWFRQAPGKEREFVAGIRWISGNPYYADSVKGRFTISRDNAKNTVYLQMNSLKPEDTAVYSCAAAPLTRSYDYWGQGTQVTVSS

SEQ ID NO: 182 - protein, 246 aa, synthetic construct
  REGION 1..246: note = homodimeric fusion protein
EVQLVESGGGLVQAGGSLRLACAASGLTFSPYAMGWFRQAPGKEREFVAGIRWISGNPYYADSVKGRFTISRDNSKNTLYLQMNSLRPEDTAVYSCAAAPLTRSYDYWGQGTLVTVSSGGGGSGGGGSEVQLVESGGGLVQAGGSLRLACAASGLTFSPYAMGWFRQAPGKEREFVAGIRWISGNPYYADSVKGRFTISRDNSKNTLYLQMNSLRPEDTAVYSCAAAPLTRSYDYWGQGTLVTVSS

SEQ ID NO: 183 - protein, 735 aa, Adeno-associated virus 2 [sequence omitted]